(19) United States
(12) Patent Application Publication
(10) Pub. No.: US 2006/0105977 A1
(43) Pub. Date: May 18, 2006

(54) Desmoglein 4 is a novel gene involved in hair growth

(76) Inventor: Angela M. Christiano, Upper Saddle River, NJ (US)

Correspondence Address: BAKER & BOTTS, 30 ROCKEFELLER PLAZA, NEW YORK, NY, 10112, US

(21) Appl. No.: 11/251,340

(22) Filed: Oct. 14, 2005

Related U.S. Application Data
(63) Continuation of application No. PCT/US04/11697, filed on Apr. 15, 2004.
(60) Provisional application No. 60/464,013, filed on Apr. 17, 2003.

(51) Int. Cl. A61K 48/00; C07H 21/02; A61K 9/127; C12N 15/88

(52) U.S. Cl. 514/44; 536/23.1; 424/450; 435/458

(57) ABSTRACT

This invention provides methods and compositions for inhibiting the expression of desmoglein 4. This invention also provides pharmaceutical compositions for inhibiting hair growth in a subject.

This application is a continuation of International Patent Application PCT/US04/011697 filed Apr. 15, 2004 and published Nov. 4, 2004 under International Publication No. WO 2004/093788, which claims the benefit of copending U.S. Provisional Application No. 60/464,013, filed Apr. 17, 2003, the contents of each of which are hereby incorporated by reference and to each of which priority is claimed.

The invention disclosed herein was made with Government support under grant number ROI 44924 from the National Institutes of Health, U.S. Department of Health and Human Services. Accordingly, the U.S. Government has certain rights in this invention.

### BACKGROUND OF THE INVENTION

Throughout this application, various publications are referenced in parentheses by name or number. Full citations for these references may be found at the end of each experimental section. The disclosures of these publications in their entireties are hereby incorporated by reference into this application to more fully describe the state of the art to which this invention pertains.

The hair follicle (HF) is among the few mammalian organs which periodically reverts to a morphogenic program of cellular events as a part of its normal cycle of growth (anagen), involution (catagen) and quiescence (telogen) (Fuchs et al., 2001; Hardy, 1992). The HF develops as the result of a series of reciprocal epithelial-mesenchymal signals between the dermal papilla (DP) and the overlying epithelium during morphogenesis. It is the transmission of morphogenic signals via elaborate networks of cell contacts during development that transforms simple sheets of epithelial cells into complex three-dimensional structures, such as the HF (Fuchs et al., 2001; Jamora and Fuchs, 2002). The cellular rearrangements that occur with each adult mouse hair cycle are no less dynamic and well-orchestrated, given that the entire population of hair matrix keratinocytes is reduplicated in approximately 13 hours (Bullough and Laurence, 1958; Van Scott et al., 1963). Keratinocytes in the lowermost HF are multipotent and proliferate rapidly until they pass through a zone parallel to the widest part of the DP, known as the “critical region” or the line of Auber (Auber, 1952) above which mitosis ceases, differentiation begins, and the gradual elongation of cells takes place as they ascend and form the concentric layers of the HF.

The determination of keratinocyte cell fate in the lower HF is governed by morphogens including bone morphogenic proteins (BMPs) and sonic hedgehog (Shh), membrane bound signaling molecules such as Notch and Delta, and secreted growth factors such as Wnts and FGFs, whose expression is active during HF morphogenesis and is reprised in each adult hair cycle (Fuchs et al., 2001; Jamora and Fuchs, 2002). The network of cell-cell junctions that provides the infrastructure for transmission of these signals is critical for imparting information to the proliferating keratinocytes to direct them down one of several specific differentiation pathways (Orwin, 1979). To meet the demand for sophisticated communication and signaling events orchestrated by cell-cell adhesion, the number of desmosomes more than doubles during differentiation (Orwin, 1979), such that in a mature HF, up to 90% of the cell surfaces of the individual keratinocyte layers within the inner root sheath (IRS) are occupied by desmosomes (Roth and Helwig, 1964). The line of Auber represents an information portal through which multipotent keratinocytes must quickly pass, receiving instructions that determine their destiny as they enter, and executing highly intricate programs of differentiation upon their exit.

Intercellular junctions are critical for orchestrating the molecular events during HF induction and cycling, which require synchronization of the transition from proliferation to differentiation (Jamora and Fuchs, 2002). Desmosomes are elaborate multiprotein complexes that link heterotypic cadherin partners to the intermediate filament (IF) network via plakin and armadillo family members (Fuchs et al., 2001; Green and Gaudry, 2000). In mouse and human, three desmoglein (DSG1, 2, 3) and three desmocollin (DSC1, 2, 3) genes have been described previously. DSG1, DSC1, DSG3 and DSC3 are predominantly expressed in stratifying squamous epithelia such as the epidermis, whereas DSG2 and DSC2 are present in simple epithelia and non-epithelial tissues as well.

In the epidermis, DSG1 and DSC1 are expressed in the suprabasal layers of the epidermis, while DSG3 and DSC3 are present in the basal layer (Garrod et al., 2002; Green and Gaudry, 2000). DSG1 and DSG3 also serve as autoantigens in the acquired bullous dermatoses, pemphigus foliaceus and pemphigus vulgaris (PV), respectively, which are characterized by loss of cell-cell adhesion in the epidermis (Green and Gaudry, 2000; McMillan and Shimizu, 2001). Desmosomes impart structural integrity to tissues undergoing mechanical stress, and recent evidence suggests that they may also regulate the availability of signaling molecules and transduce signals that dictate the state of the cytoskeleton and activate downstream genetic programs (Fuchs et al., 2001; Green and Gaudry, 2000). The critical role of the desmosomal proteins in epithelial integrity has been illustrated by targeted ablation of the corresponding genes in mice, as well as their disruption in human diseases. The phenotypes that arise in these mice range from embryonic lethal, such as Dsg2, desmoplakin (Dsp), and plakoglobin (Pg) (Eshkind et al., 2002; Jamora and Fuchs, 2002), to relatively mild, as in Dsc1 null animals (Chidgey et al., 2001), or Dsg3 null animals which are. allelic to the spontaneous, cyclical balding mouse (Koch et al., 1997; Montagutelli et al., 1997; Pulkkinen et al., 2002). Non-lethal mutations in the genes encoding desmosomal proteins have also been identified in humans (McMillan and Shimizu, 2001). With the exception of DSG1, these disorders are unified by profound abnormalities in the HF. For example, mutations in DSP and PG underlie Naxos disease, characterized by woolly, sparse hair, keratoderma and cardiomyopathy (McKoy et al., 2000; Norgett et al., 2000), plakophilin 1 (PKP1) mutations cause ectodermal dysplasia with sparse hair and skin fragility (McGrath et al., 1997), and keratodermas result from mutations in either DSG1 or DSP (Armstrong et al., 1999; Hunt et al., 2001; Kljuic et al., In Press). While these models have provided significant insights into the role of intercellular adhesion proteins in epidermal cytoarchitecture in either mouse or human, examples have not yet emerged of desmosomal proteins for which direct parallels between a human genetic disease, an acquired autoimmune disease, and corresponding mouse models can be drawn.

It is puzzling that despite the preponderance of desmosomes in the inner layers of the hair shaft, and their critical role in intercellular adhesion, none of the known desmosomal cadherin genes are highly expressed in this region (Koch et al., 1997; Kurzen et al., 1998).

Using a classical genetic approach, we discovered a fourth member of the desmosomal cadherin gene superfamily, desmoglein 4 (DSG4), which is expressed in both the suprabasal epidermis and extensively throughout the matrix, precortex, and IRS of the HF. We identified causative mutations in desmoglein 4 underlying both an inherited form of human hypotrichosis, and both of the lanceolate mouse models. Further, we show that DSG4 serves as an autoantigen in the sera of patients with PV.

Characterization of the phenotype of mutant mouse epidermis revealed a hyperproliferative phenotype, including suprabasal expression of 91 integrin and ectopically proliferating cells. In the lower HF, we discovered a premature switch from proliferation to differentiation, as well as perturbations in the onset of hair shaft differentiation programs. Our findings establish a central role for desmoglein 4 in epidermal cell adhesion, and in coordinating the transition from proliferation to differentiation in HF keratinocytes, and disclose inhibition of Desmoglein 4 can cause inhibition of hair growth.

### SUMMARY OF THE INVENTION

This invention provides a catalytic deoxyribonucleic acid molecule that specifically cleaves a mRNA encoding Desmoglein 4 comprising:

(a) a catalytic domain that cleaves mRNA at a defined consensus sequence;

(b) a binding domain contiguous with the 5′ end of the catalytic domain; and

(c) a binding domain contiguous with the 3′ end of the catalytic domain,

wherein the binding domains are complementary to, and therefore hybridize with, the two regions flanking the defined consensus sequence within the mRNA encoding Desmoglein 4 at which cleavage is desired, and wherein each binding domain is at least 4 residues in length and both binding domains have a combined total length of at least 8 residues.

This invention also provides a catalytic ribonucleic acid molecule that specifically cleaves a mRNA encoding Desmoglein 4 comprising:

(a) catalytic domain that cleaves mRNA at a defined consensus sequence;

(b) a binding domain contiguous with the 5′ end of the catalytic domain; and

(c) a binding domain contiguous with the 3′ end of the catalytic domain,

wherein the binding domains are complementary to, and therefore hybridize with, the two regions flanking the defined consensus sequence within the mRNA encoding Desmoglein 4 at which cleavage is desired, and wherein each binding domain is at least 4 residues in length and both binding domains have a combined total length of at least 8 residues. This invention also provides a pharmaceutical composition comprising the instant catalytic nucleic acid molecules and a pharmaceutically acceptable carrier.

This invention also provides a method of specifically cleaving an mRNA encoding Desmoglein 4 comprising contacting the mRNA with any of the instant catalytic nucleic acid molecules under conditions permitting the molecule to cleave the mRNA.

This invention also provides a method of specifically cleaving an mRNA encoding Desmoglein 4 in a cell, comprising contacting the cell containing the mRNA with any of the instant catalytic nucleic acid molecules so as to specifically cleave the mRNA encoding Desmoglein 4 in the cell.

This invention also provides a method of specifically inhibiting the expression of Desmoglein 4 in a cell that would otherwise express Desmoglein 4, comprising contacting the cell with any of the instant catalytic nucleic acid molecules so as to specifically inhibit the expression of Desmoglein 4 in the cell.

This invention also provides a method of specifically inhibiting the expression of Desmoglein 4 in a subject's cells comprising administering to the subject an amount of any of the instant catalytic nucleic acid molecules effective to specifically inhibit the expression of Desmoglein 4 in the subject's cells.

This invention also provides a method of specifically inhibiting the expression of Desmoglein 4 in a subject's cells comprising administering to the subject an amount of any of the instant pharmaceutical compositions effective to specifically inhibit the expression of Desmoglein 4 in the subject's cells.

This invention also provides a method of inhibiting hair production by a hair-producing cell comprising contacting the cell with an effective amount of any of the instant catalytic nucleic acid moelcules.

This invention also provides a method of inhibiting hair growth in a subject comprising administering to the subject an effective amount of any of the instant pharmaceutical compositions.

This invention also provides a method of inhibiting the transition of a hair follicle from proliferation to differentiation comprising contacting the follicle with an effective amount of any of the instant catalytic nucleic acid molecules.

This invention also provides a method of inhibiting the transition of a hair follicle from proliferation to the differentiation comprising contacting the follicle with an effective amount of any of the instant pharmaceutical compositions.

This invention also provides a vector which comprises a sequence encoding any of the instant catalytic nucleic acid molecules. This invention also provides a host-vector system comprising a cell having the instant vector therein.

This invention also provides a method of producing the instant catalytic nucleic acid molecules comprising culturing a cell having therein a vector comprising a sequence encoding said catalytic nucleic acid molecule under conditions permitting the expression of the catalytic nucleic acid molecule by the cell.

This invention also provides a nucleic acid molecule that specifically hybridizes to an mRNA encoding Desmoglein 4 so as to inhibit the translation thereof in a cell.

This invention provides a non-human transgenic mammal, wherein the mammal's genome:

(a) has stably integrated therein a nucleotide sequence encoding a human Desmoglein 4 operably linked to a promoter, whereby the nucleotide sequence is expressed; and

(b) lacks an expressible endogenous Desmoglein 4 encoding nucleic acid sequence.

This invention provides a oligonucleotide comprising consecutive nucleotides that hybridizes with a Desmoglein 4-encoding mRNA under conditions of high stringency and is between 8 and 40 nucleotides in length.

This invention provides a pharmaceutical composition comprising (a) the instant oligonucleotide and (b) a pharmaceutically acceptable carrier.

This invention provides a method of treating a subject which comprises administering to the subject an amount of the instant oligonucleotide effective to inhibit expression of a Desmoglein 4 in the subject so as to thereby treat the subject.

This invention provides a method of specifically inhibiting the expression of Desmoglein 4 in a cell that would otherwise express Desmoglein 4, comprising contacting the cell with the instant oligonucleotide so as to specifically inhibit the expression of Desmoglein 4 in the cell.

This invention provides a method of specifically inhibiting the expression of Desmoglein 4 in a subject's cells comprising administering to the subject an amount of the instant oligonucleotide effective to specifically inhibit the expression of Desmoglein 4 in the subject's cells.

This invention provides a method of specifically inhibiting the expression of Desmoglein 4 in a subject's cells comprising administering to the subject an amount of the instant pharmaceutical composition effective to specifically inhibit the expression of Desmoglein 4 in the subject's cells.

This invention provides a method of inhibiting hair production by a hair-producing cell comprising contacting the cell with an effective amount of the instant oligonucleotide.

This invention provides a method of inhibiting hair growth in a subject comprising administering to the subject an effective amount of the instant pharmaceutical composition. In one embodiment the subject is a mammal. In one embodiment the mammal is a human being.

### BRIEF DESCRIPTION OF THE FIGURES

FIG. 1. Linkage analysis in LAH pedigrees: (A, B) Haplotypes for chromosome 18 markers are shown for representative individuals in pedigrees LAH-1 (A) and LAH-2 (B). The key recombination event in IV-10 between markers D18S1149 and D18S1108 is indicated by an arrow (A). Filled symbols designate affected individuals and consanguinous loops are indicated by double lines. Microsatellite markers are boxed and the disease-associated haplotype is shaded. (C) Two-point LOD scores for chromosome 18 markers in the two LAH pedigrees combined. Values higher than 3 are underlined. The position for each marker is indicated in centimorgans (cM), according to the Marshfield genetic map (see the Marshfield Clinic website). (D) Multipoint LOD scores in the two LAH pedigrees combined. The relative position of each marker in cM and the LOD score values are indicated on the X and Y-axis, respectively.

FIG. 2. Clinical and histological features of the human LAH phenotype and the lanceolate hair mouse: (A-D) Clinical presentation of the human LAH phenotype in family LAH-1 (A, B) and LAH-2 (C, D). Note the sparse scalp hair and eyebrows (A, B) and bumpy scalp skin (C, D). (E-H) Gross abnormalities in the lanceolate hair mice. (F) Day 13 lah/lah male, with sparse hair on the trunk and abnormal vibrissae. (E) A wild-type day 13 PWK littermate. (G) Day 14 DBA/llahJ+/+ (left) and lahJ/lahJ (right) male mice. (H) The mutant mouse is bald, runted, and has thickened, folded skin. Vibrissae are completely absent. (I-L) Skin histology (H & E) from affected patients (I, K) and day 8 lahJ/lahJ (J, L). The formation of a bulbous“bleb” (I, J) and the presence of curled ingrown hair shafts within the hair follicle (K, L) are observed in both human and mouse. Hyperplastic interfollicular epidermis and HF infundibulum are observed in lahJ/lahJ skin (L), but not in human LAH skin (K). (M) Hair fiber emerging from the skin of a 2 month old DBA/llacJ lahJ/lahJ mutant female. Note the bulbous swelling at the tip where the fiber has broken off (arrow). The adjacent anagen hair follicles all have bulbous degenerative changes (arrowheads). (N) Bright field illumination of lah/lah hairs. Note the bulbous degenerative changes at the breakpoint in the hair. Scale bars: 1, M-75 mm; J-40 mm; K-100 mm; L-60 mm.

FIG. 3. Genomic organization and expression analysis of desmoglein 4: (A) Genomic structure of the human (top) and mouse (bottom) desmosomal cadherin gene clusters on chromosome 18. The approximate size of genes and intragenic regions are indicated in kb, according to the UCSC freezes of December 01 (human) and February 02 (mouse). (B) Amino acid sequence alignment of representative fragments of human DSG1 (SEQ ID NO: 23), human DSG2 (SEQ ID NO: 24), human DSG3 (SEQ ID NO: 25) and human DSG4 (SEQ ID NO:1). The peptide sequence against which the antibody was raised is boxed. Asterisks indicate identical residues. (C) Amino acid identity and homology between DSG1-4. GenBank accession numbers for DSG4 and Dsg4 are AY227350 and AY227349. (D) Comparison of domains among human desmoglein proteins. Note the highly conserved protein structure among all desmogleins, with the exception of the RUD. EI-EIV, extracellular cadherin repeats; EA, extracellular anchor domain; TM, transmembrane domain; IA, intracellular anchor; ICS, intracellular cadherin sequence; LD, intracellular linker domain; RUD, repeated unit domain; TD, terminal domain. (E-F) Northern analysis of human (E) and mouse (F) desmoglein 4.

FIG. 4. Mutation analysis in human and mouse desmoglein 4: (A-B) DSG4 deletion in patients from pedigrees LAH-1 and LAH-2. (A) The deletion breakpoint is between introns 4 and 8 in both LAH pedigrees (B). Schematic representation of the deletion in LAH patients. The size of the introns is in kb. (C-G) Dsg4 mutations in LahJ/lahJ (C, D) and lah/lah (E, G). (C). A DNA sequencing tracing shows WT/WT mouse sequence CTGTCC; (SEQ ID NO: 38) and corresponding LahJ/LahJ sequence CTTGTCC (SEQ ID NO: 39). LahJ/lahJ mice were homozygous for a 1-bp insertion in exon 7 (SEQ ID NO: 28) (C), which creates a frameshift and premature termination codon three codons downstream (SEQ ID NO: 29) (D). WT/WT indicates the wild-type nucleotide sequence (SEQ ID NO: 26) with the corresponding wild-type amino acid sequence (SEQ ID NO: 27). (E) Sequence analysis of Dsg4 in lah/lah mice revealed a homozygous missense mutation, Y196S, in exon 6. (G) Y196 is conserved among different cadherin proteins. Shown is an alignment of amino acid sequence spanning Y196 in Dsg4 (SEQ ID NO: 30); DSG4 (SEQ ID NO: 31); Dsg2 (SEQ ID NO: 32); Dsc1 (SEQ ID NO: 33); Dsc 3 (SEQ ID NO: 34); E-cad (SEQ ID NO: 35) and N-cad (SEQ ID NO: 36). The conserved tyrosine is boxed. (F) RT-PCR analysis of skin mRNA from lahJ/lahJ and lah/lah showed presence of the mutant transcript in lah/lah, but complete lack of Dsg4 expression in lahJ/lahJ. Amplification of Dsg3 is shown on the lower panel for comparison. Lanes: 1, marker; 2 and 3, PWK+/+; 4 and 6, lah/lah; 5 and 7, lahJ/lahJ. 8, blank control.

FIG. 5. Desmoglein 4 expression and ultrastructural defects in lahJ/lahJ skin: (A) In situ hybridization of mouse Dsg4 in vibrissa shows a strong signal in the upper matrix. (B) Control sense probe. (C) Immunofluorescence staining of human DSG4 in dissected human scalp follicle shows intense staining in the IRS and all layers of the matrix and precortex. (D) In contrast, DSG1 expression is localized only to the IRS. (E, F) DSG4 immunostaining in interfollicular epidermis reveals a strong positive signal in the suprabasal layers. (G) PV autoantibodies recognize DSG4. Lanes 1 and 2 were stained with sera from a healthy male and female subjects with no history of skin disease. Lanes 3 and 4 were stained with sera from two different PV patients with active lesions at the time serum was obtained. Sera recognize a recombinant protein of N-terminal region of DSG4 (42 kD). (H-0) Dysadhesion between all keratinocyte layers in day 14 mutant epidermis (H) compared to WT epidermis (I) with tight adhesion between cells (4, 000,×). (J) Loss of connection between four adjoining suprabasal keratinocytes reveals sparse poorly formed desmosomes between cells, with scant insertion of filaments as compared to WT cells (K) (7, 500×). (L) High magnification of desmosomes that have been torn away from adjacent keratinocytes compared to intact desmosomes (M) in WT skin (15, 000×). (N) Disorganization of the medulla in the area just above the dermal papilla in a day 14 lahJ/lahJ mutant animal, while the concentric layers of the hair shaft and IRS still appear largely normal (2,500×). (O) Higher up the HF, adjoining keratinocytes within the IRS layers are now torn apart, leaving behind rows of desmosomes along previously adherent cell borders (arrows) (4, 000×). 0-outer root sheath; M-medulla; Co-cortex; Cu-Cuticle of cortex; Hx-Huxley's layer; He-Henle's s layer. White dashed lines demarcate the dermal-epidermal junction. Scale bars: A, C, F, G-100 mm; B,-50 mm; D-60 mm; F-10 mm.

FIG. 6. Activation of epidermal keratinocytes in lah/lah and lahJ/lahJ mutant skin: (A-H) Comparison of different proliferation and differentiation markers between day 8 lahJ/lahJ and WT epidermis. (A, B) K5 immunofluorescence shows patchy expression in basal cells of lahJ/lahJ epidermis. K6 is ubiquitously expressed in lahJ/lahJ epidermis and infundibulum of HF (C), while WT epidermis is negative (D). (E, F) a6 integrin, a hemidesmosomal component, is markedly reduced in lahJ/lahJ basal epidermis. (G, H) PCNA immunohistochemistry shows a higher number of positive staining cells in the thickened (brackets) lahJ/lahJ epidermis (G) compared to WT (H). Suprabasal β integrin (I, J) and EGFR (K, L) in mutant versus WT epidermis. lah/lah epidermal keratinocytes exhibit enhanced attachment and spreading after 24 hrs in culture (M) relative to WT keratinocytes (N). (O) Quantitative measurement of the fraction of attached cells in M and N. Error bar: standard error of the mean (SEM). White dashed lines demarcate the dermal-epidermal junction. Scale bars: A, B, G, H-32 mm; C, D, E, F-40 mm; 1, J, K, L-2.5 mm.

FIG. 7. lahJ/lahJ hair matrix keratinocytes display perturbations in the switch from proliferation to differentiation: (A, B) PCNA immunohistochemistry reveals an abrupt transition from proliferation (brown) to differentiation (blue) as compared to the gradual transition in a WT follicle. This occurs in a region of cell-cell separation (C) compared to the tight adhesion between cells of a WT follicle (D). (E) Schematic of HF showing the concentric layers and keratinization patterns. (F-K) Downregulation and misexpression of hair keratins and hoxC13. HoxC13 expression is reduced in lahJ/lahJ matrix/precortex cells (F) compared to WT skin (G). hHb2 (H, I) and hHa4 (J, K), hair keratins specific for hair shaft cuticle and cortex, respectively, show spatially reduced expression in lahJ/lahJ follicles. White dashed lines demarcate the dermal-epidermal junction. Scale bars: A, B, C, D-20 mm; F-45 mm.

FIG. 8A-C. Human Desmoglein 4 protein sequence (SEQ ID NO: 1) and cDNA (SEQ ID NO: 2).

FIG. 9A-C. Mouse Desmoglein 4 protein sequence (SEQ ID NO: 3) and cDNA (SEQ ID NO: 4).

### DETAILED DESCRIPTION OF THE INVENTION

### DEFINITIONS

As used herein, and unless stated otherwise, each of the following terms shall have the definition set forth below.

“Administering” shall mean administering according to any of the various methods and delivery systems known to those skilled in the art. The administering can be performed, for example, via implant, transmucosally, transdermally and subcutaneously. In the preferred embodiment, the administering is topical and preferably dermal.

“Catalytic” shall mean the functioning of an agent as a catalyst, i.e. an agent that increases the rate of a chemical reaction without itself undergoing a permanent structural change.

“Consensus sequence” shall mean a nucleotide sequence of at least two residues in length between which catalytic nucleic acid cleavage occurs. For example, consensus sequences include “A:C” and “G:U”.

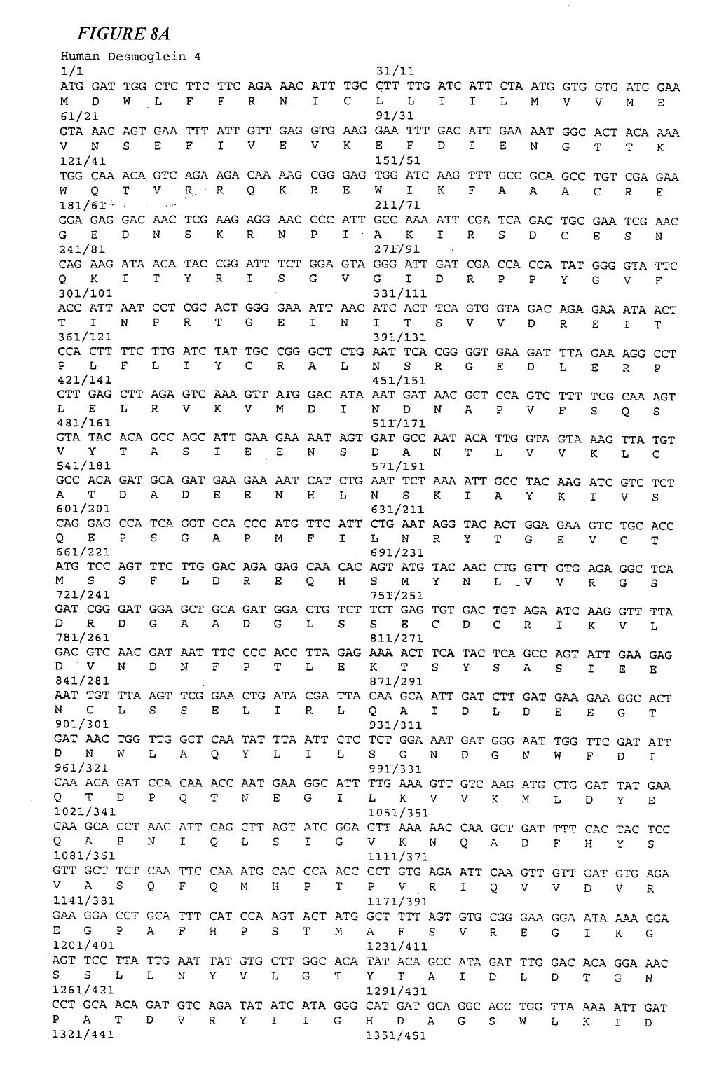

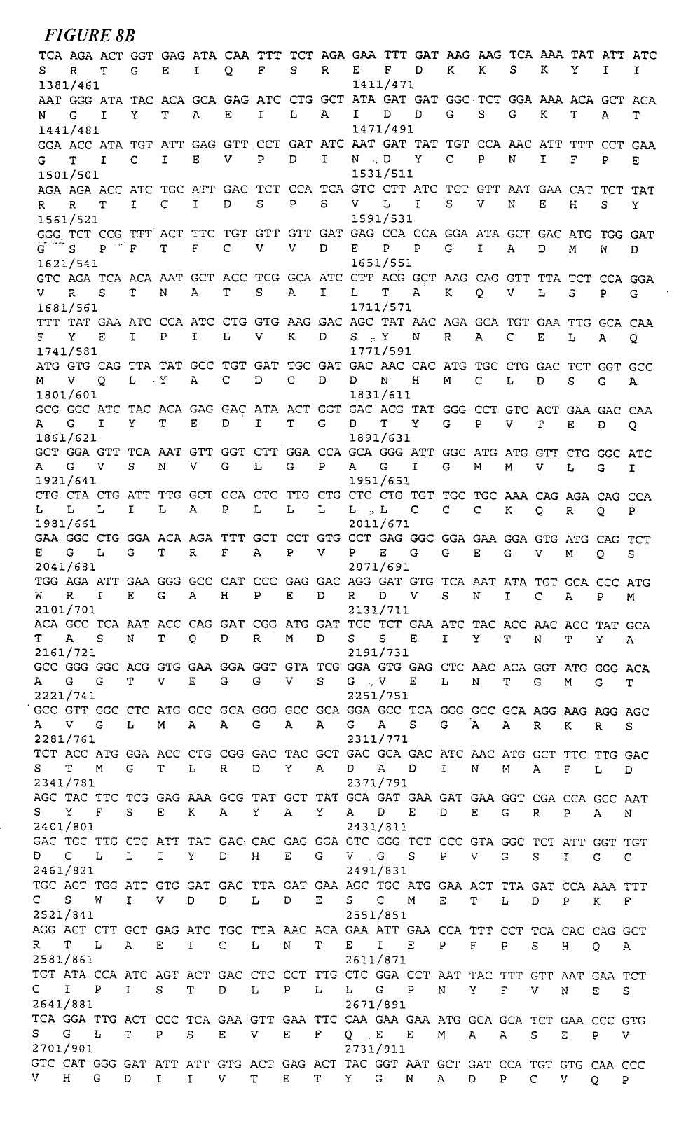

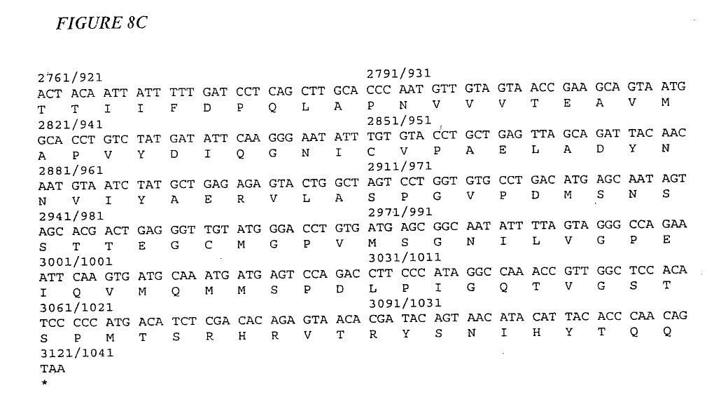

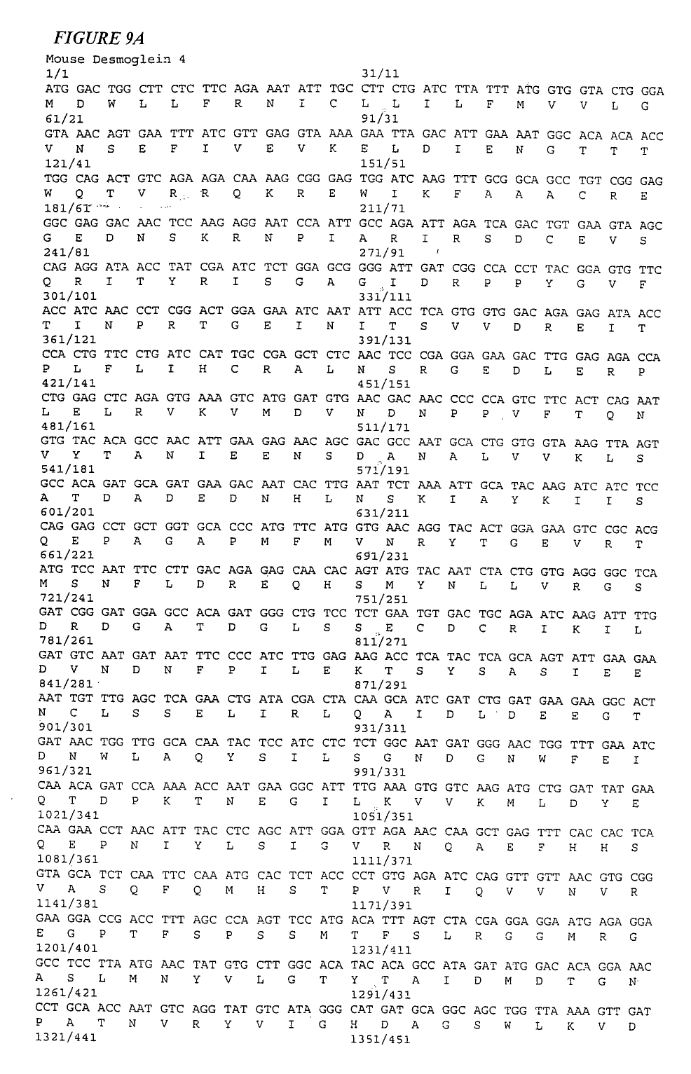

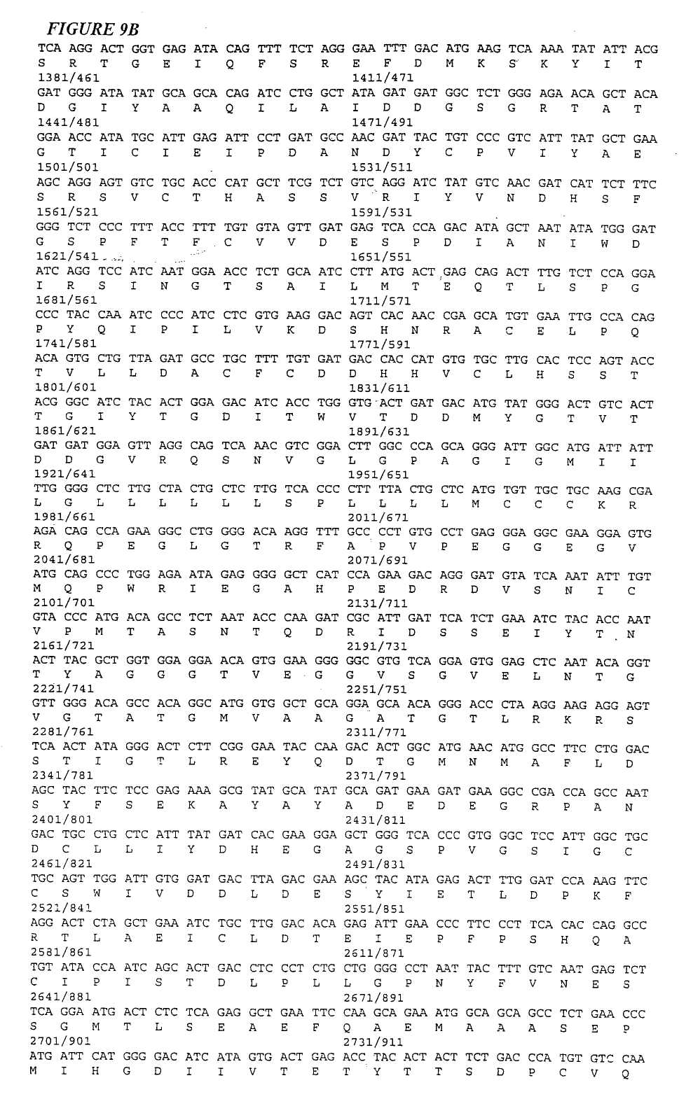

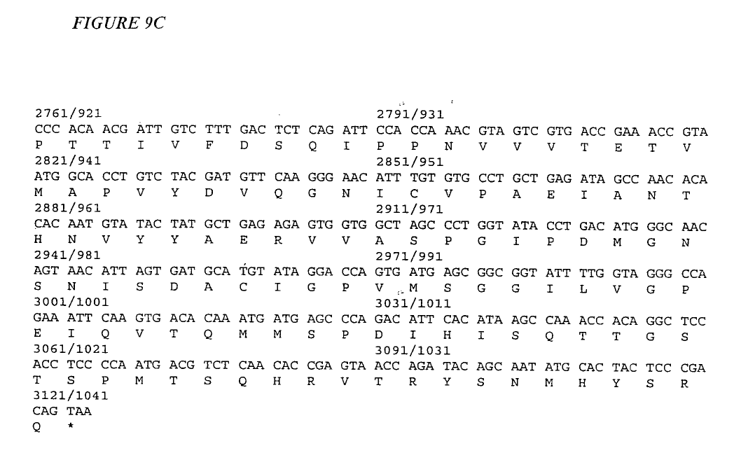

“Desmoglein 4” shall mean the protein encoded by the nucleotide sequence shown in FIGS. 8A-8C (SEQ ID NO: 2) when human and the nucleotide sequence shown in FIGS. 9A-9C (SEQ ID NO: 4) when murine, and having the amino acid sequence shown in SEQ ID NO: 1 or 3 respectively, or homologs, and any variants thereof, whether artificial or naturally occurring. Variants include, without limitation, homologues, post-translational modifications, mutants and polymorphisms. Sequence identity between variants is the similarity between two nucleic acid sequences, or two amino acid sequences is expressed in terms of the similarity between the sequences, otherwise referred to as sequence identity. Sequence identity is frequently measured in terms of percentage identity (or similarity or homlogy); the higher the percentage, the more similar the two sequences are. Homologs of the human and mouse Desmoglein 4 proteins will possess a relatively high degree of sequence identity when aligned using standard methods.

Methods of alignment of sequences for comparison are well-known in the art. Various programs and alignment algorithms are described which present a detailed consideration of sequence alignment methods and homology calculations. Additionally, the NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be, accessed at the NCBI online site under the “BLAST” heading. A description of how to determine sequence identity using this program is available at the NCBI online site under the “BLAST overview” subheading.

Homologs of the disclosed Desmoglein 4 are typically characterized by possession of at least 70% sequence identity counted over the full length alignment with the disclosed amino acid sequence of either the human or mouse Desmoglein 4 amino acid sequences using the NCBI Blast 2.0, gapped blastp set to default parameters. Proteins with even greater similarity to the reference sequences will show increasing percentage identities when assessed by this method, such as at least 75%, at least 80%, at least 90% or at least 95% sequence identity. When less than the entire sequence is being compared for sequence identity, homologs will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI online site under the “Frequently Asked Questions” subheading. One of skill in the art will appreciate that these sequence identity ranges are provided for guidance only; it is entirely possible that strongly significant homologs could be obtained that fall outside of the ranges provided. The present invention provides not only the peptide homologs are described above, but also nucleic acid molecules that encode such homologs.

One indication that two nucleic acid sequences are substantially identical is that the polypeptide which the first nucleic acid encodes is immunologically cross reactive with the polypeptide encoded by the second nucleic acid. Another indication that two nucleic acid sequences are substantially identical is that the two molecules hybridize to each other under stringent conditions. Stringent conditions are sequence dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (1989).

Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). One or more factors be may be varied to generate conditions of either low or high stringency different from, but equivalent to, the above listed conditions. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequence that all encode substantially the same protein.

“Desmoglein 4-encoding mRNA” shall mean, unless otherwise indicated, any mRNA molecule comprising a sequence which encodes Desmoglein 4. Desmoglein 4-encoding mRNA includes, without limitation, protein-encoding sequences as well as the 5 ′ and 3′ non-protein-encoding sequences.

“Hybridize” shall mean the annealing of one single-stranded nucleic acid molecule to another nucleic acid molecule based on sequence complementarity. The propensity for hybridization between nucleic acids depends on the temperature and ionic strength of their milieu, the length of the nucleic acids and the degree of complementarity. The effect of these parameters on hybridization is well known in the art (see Sambrook, 1989). Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al. (1989), chapters 9 and 11, herein incorporated by reference.

“Inhibit” shall mean to slow, or otherwise impede.

“Nucleic acid molecule” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Pharmaceutically acceptable carrier” shall mean any of the various carriers known to those skilled in the art. In one embodiment, the carrier is an alcohol, preferably ethylene glycol. In another embodiment, the carrier is a liposome. The following pharmaceutically acceptable carriers are set forth, in relation to their most commonly associated delivery systems, by way of example, noting the fact that the instant pharmaceutical compositions are preferably delivered dermally.

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N, NI, NII, NIII-tetramethyl-N, NI, NII, NIII-tetrapalmity-spermine and dioleoyl phosphatidylethanol-amine (DOPE) (GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA and the neutral lipid DOPE (GIBCO BRL).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

“Specifically cleave”, when referring to the action of one of the instant catalytic nucleic acid molecules on a target mRNA molecule, shall mean to cleave the target mRNA molecule without cleaving another mRNA molecule lacking a sequence complementary to either of the catalytic nucleic acid molecule's two binding domains.

“Subject” shall mean any animal, such as a human, a primate, a mouse, a rat, a guinea pig or a rabbit.

“Vector” shall include, without limitation, a nucleic acid molecule that can be used to stably introduce a specific nucleic acid sequence into the genome of an organism.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Finally, the following abbreviations shall have the meanings set forth below: “A” shall mean Adenine; “bp” shall mean base pairs; “C” shall mean Cytosine; “DNA” shall mean deoxyribonucleic acid; “G” shall mean Guanine; “mRNA” shall mean messenger ribonucleic acid; “RNA” shall mean ribonucleic acid; “RT-PCR” shall mean reverse transcriptase polymerase chain reaction; “RY” shall mean purine: pyrimidine; “T” shall mean Thymine; and “U” shall mean Uracil.

### Embodiments of the Invention

This invention provides a catalytic deoxyribonucleic acid molecule that specifically cleaves a mRNA encoding Desmoglein 4 comprising:

(a) a catalytic domain that cleaves mRNA at a defined consensus sequence;

(b) a binding domain contiguous with the 5 ′ end of the catalytic domain; and

(c) a binding domain contiguous with the 3 ′ end of the catalytic domain,

wherein the binding domains are complementary to, and therefore hybridize with, the two regions flanking the defined consensus sequence within the mRNA encoding Desmoglein 4 at which cleavage is desired, and wherein each binding domain is at least 4 residues in length and both binding domains have a combined total length of at least 8 residues. In a preferred embodiment, each binding domain is 7 residues in length, and both binding domains have a combined total length of 14 residues.

The catalytic domain may optionally contain stem-loop structures in addition to the nucleotides required for catalytic activity. In one embodiment the catalytic domain has the sequence ggctagctacaacga (SEQ ID NO: 5), and cleaves mRNA at the consensus sequence purine:pyrimidine.

This invention also provides a catalytic ribonucleic acid molecule that specifically cleaves a mRNA encoding Desmoglein 4 comprising:

(a) catalytic domain that cleaves mRNA at a defined consensus sequence;

(b) a binding domain contiguous with the 5 ′ end of the catalytic domain; and

(c) a binding domain contiguous with the 3 ′ end of the catalytic domain,

wherein the binding domains are complementary to, and therefore hybridize with, the two regions flanking the defined consensus sequence within the mRNA encoding Desmoglein 4 at which cleavage is desired, and wherein each binding domain is at least 4 residues in length and both binding domains have a combined total length of at least 8 residues.

In one embodiment of the instant catalytic ribonucleic acid molecule, each binding domain is at least 12 residues in length. In the preferred embodiment, each binding domain is no more than 17 residues in length. In another embodiment, both binding domains have a combined total length of at least 24 residues, and no more than 34 residues.

In one embodiment the instant catalytic ribonucleic acid molecule is a hammerhead ribozyme. Hammerhead ribozymes are well known in the literature, as described in Pley et al., 1994. In one embodiment, the consensus sequence is the sequence 5′-NUH-3′, where N is any nucleotide, U is uridine and H is any nucleotide except guanine. An example of such sequence is 5′-adenin:uracil:adenine-3′. In another embodiment, the catalytic domain has the sequence ctgatgagtccgtgaggacgaaaca (SEQ ID NO: 6).

In an alternative embodiment of the instant catalytic ribonucleic acid molecule, the molecule is a hairpin ribozyme. Hairpin ribozymes are well known in the literature as described in Fedor (2000).

This invention further provides the instant catalytic nucleic acid molecules, wherein the Desmoglein 4 comprises consecutive amino acids having the sequence set forth in SEQ ID NO: 1 or SEQ ID NO: 3.

This invention further provides, the instant catalytic nucleic acid molecules, wherein the Desmoglein 4 encoding mRNA comprises consecutive nucleotides having the sequence set forth in SEQ ID NO: 2 or SEQ ID NO: 4.

This invention further provides the instant catalytic nucleic acid molecules, wherein the cleavage site within the mRNA encoding Desmoglein 4 is located within the first 3000 residues following the mRNA's 5′ terminus.

This invention further provides the instant catalytic nucleic acid molecules, wherein the cleavage site within the mRNA encoding Desmoglein 4 is located within the first 1500 residues following the mRNA's 5′ terminus.

This invention further provides the instant catalytic nucleic acid molecules, wherein the mRNA encoding Desmoglein 4 is from a subject selected from the group consisting of human, monkey, rat and mouse.

This invention also provides a pharmaceutical composition comprising the instant catalytic nucleic acid molecules and a pharmaceutically acceptable carrier. In one embodiment the carrier is an alcohol. In one embodiment the carrier is ethylene glycol. In one embodiment the carrier is a liposome.

This invention also provides a method of specifically cleaving an mRNA encoding Desmoglein 4 comprising contacting the mRNA with any of the instant catalytic nucleic acid molecules under conditions permitting the molecule to cleave the mRNA.

This invention also provides a method of specifically cleaving an mRNA encoding Desmoglein 4 in a cell, comprising contacting the cell containing the mRNA with any of the instant catalytic nucleic acid molecules so as to specifically cleave the mRNA encoding Desmoglein 4 in the cell.

This invention also provides a method of specifically inhibiting the expression of Desmoglein 4 in a cell that would otherwise express Desmoglein 4, comprising contacting the cell with any of the instant catalytic nucleic acid molecules so as to specifically inhibit the expression of Desmoglein 4 in the cell.

This invention also provides a method of specifically inhibiting the expression of Desmoglein 4 in a subject's cells comprising administering to the subject an amount of any of the instant catalytic nucleic acid molecules effective to specifically inhibit the expression of Desmoglein 4 in the subject's cells.

This invention also provides a method of specifically inhibiting the expression of Desmoglein 4 in a subject's cells comprising administering to the subject an amount of any of the instant pharmaceutical compositions effective to specifically inhibit the expression of Desmoglein 4 in the subject's cells.

A method of inhibiting hair production by a hair-producing cell comprising contacting the cell with an effective amount of any of the instant catalytic nucleic acid molecules.

A method of inhibiting hair growth in a subject comprising administering to the subject an effective amount of any of the instant pharmaceutical compositions.

A method of inhibiting the transition of a hair follicle from the proliferation phase to the differentiation phase comprising contacting the follicle with an effective amount of any of the instant catalytic nucleic acid molecules.

A method of inhibiting the transition of a hair follicle from proliferation to the differentiation comprising contacting the follicle with an effective amount of any of the instant pharmaceutical compositions.

In one embodiment of the instant methods the cell is a keratinocyte. In one embodiment of the instant methods the subject is a human. In one embodiment of the instant methods the catalytic nucleic acid molecule is administered topically. In one embodiment of the instant methods, the catalytic nucleic acid is administered dermally. In one embodiment of the instant methods the pharmaceutical composition is administered topically. In one embodiment of the instant methods the pharmaceutical composition is administered dermally.

Cleaving of Desmoglein 4-encoding mRNA with catalytic nucleic acids interferes with one or more of the normal functions of Desmoglein 4-encoding mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA.

The nucleotides may comprise other bases such as inosine, deoxyinosine, hypoxanthine may be used. In addition, isoteric purine 2′deoxy-furanoside analogs, 2′-deoxynebularine or 2′deoxyxanthosine, or other purine or pyrimidine analogs may also be used. By carefully selecting the bases and base analogs, one may fine tune the hybridization properties of the oligonucleotide. For example, inosine may be used to reduce hybridization specificity, while diaminopurines may be used to increase hybridization specificity.

Adenine and guanine may be modified at positions N3, N7, N9, C2, C4, C5, C6, or C8 and still maintain their hydrogen bonding abilities. Cytosine, thymine and uracil may be modified at positions N1, C2, C4, C5, or C6 and still maintain their hydrogen bonding abilities. Some base analogs have different hydrogen bonding attributes than the naturally occurring bases. For example, 2-amino-2′-dA forms three (3), instead of the usual two (2), hydrogen bonds to thymine (T). Examples of base analogs that have been shown to increase duplex stability include, but are not limited to, 5-fluoro-2′-dU, 5-bromo-2′-dU, 5-methyl-2′-dC, 5-propynyl-2′-dC, 5-propynyl-2′-dU, 2-amino-2′-dA, 7-deazaguanosine, 7-deazadenosine, and N2-Imidazolylpropyl-2′-dG.

Nucleotide analogs may be created by modifying and/or replacing a sugar moiety. The sugar moieties of the nucleotides may also be modified by the addition of one or more substituents. For example, one or more of the sugar moieties may contain one or more of the following substituents : amino, alkylamino, aralkyl, heteroalkyl, heterocycloalkyl, aminoalkylamino, O, H, an alkyl, polyalkylamino, substituted silyl, F, Cl, Br, CN, CF3, OCF3, OCN, 0-alkyl, S-alkyl, SOMe, SO2Me, ONOz, NH-alkyl, OCH2CH═CH2, OCHaCCH, OCCHO, allyl, O-allyl, NO2, N3, and NH2. For example, the 2′ position of the sugar may be modified to contain one of the following groups: H, OH, OCN, 0-alkyl, F, CN, CF3, allyl, 0-allyl, OCF3, S-alkyl, SOMe, SO2Me, ONO2, NO2, N3, NH2, NH-alkyl, or OCH═CH2, OCCH, wherein the alkyl may be straight, branched, saturated, or unsaturated. In addition, the nucleotide may have one or more of its sugars modified and/or replaced so as to be a ribose or hexose (i.e., glucose, galactose) or have one or more anomeric sugars. The nucleotide may also have one or more L-sugars.

Representative United States patents that teach the preparation of such modified bases/nucleosides/nucleotides include, but are not limited to, U.S. Pat. Nos. 6,248,878, and 6,251,666 which are herein incorporated by reference.

The sugar may be modified to contain one or more linkers for attachment to other chemicals such as fluorescent labels. In an embodiment, the sugar is linked to one or more aminoalkyloxy linkers. In another embodiment, the sugar contains one or more alkylamino linkers. Aminoalkyloxy and alkylamino linkers may be attached to biotin, cholic acid, fluorescein, or other chemical moieties through their amino group.

Nucleotide analogs or derivatives may have pendant groups attached. Pendant groups serve a variety of purposes which include, but are not limited to, increasing cellular uptake of the molecule, enhancing degradation of the target nucleic acid, and increasing hybridization affinity. Pendant groups can be linked to the binding domains of the catalytic nucleic acid. Examples of pendant groups include, but are not limited to: acridine derivatives (i.e. 2-methoxy-6-chloro-9-aminoacridine); cross-linkers such as psoralen derivatives, azidophenacyl, proflavin, and azidoproflavin; artificial endonucleases; metal complexes-such as EDTA-Fe (II), o-phenanthroline-Cu (1), and porphyrin-Fe (II); alkylating moieties; nucleases such as amino-1-hexanolstaphylococcal nuclease and alkaline phosphatase; terminal transferases; abzymes; cholesteryl moieties; lipophilic carriers; peptide conjugates; long chain alcohols; phosphate esters; amino; mercapto groups; radioactive markers; nonradioactive markers such as dyes; and polylysine or other polyamines. In one example, the nucleic acid comprises an oligonucleotide conjugated to a carbohydrate, sulfated carbohydrate, or gylcan. Conjugates may be regarded as a way as to introduce a specificity into otherwise unspecific DNA binding molecules by covalently linking them to a selectively hybridizing oligonucleotide.

The binding domains of the catalytic nucleic acid may have one or more of their sugars modified or replaced so as to be ribose, glucose, sucrose, or galactose, or any other sugar. Alternatively, they may have one or more sugars substituted or modified in its 2′ position, i.e. 2′ allyl or 2′-0-allyl. An example of a 2′-O-allyl sugar is a 2′-O-methylribonucleotide. Further, the nucleotides of the binding domain may have one or more of their sugars substituted or modified to form an α-anomeric sugar.

A catalytic nucleic acid binding domain may include non-nucleotide substitution. The non-nucleotide substitution includes either abasic nucleotide, polyether, polyamine, polyamide, peptide, carbohydrate, lipid or polyhydrocarbon compounds. The term “abasic” or “abasic nucleotide” as used herein encompasses sugar moieties lacking a base or having other chemical groups in place of base at the 1′ position.

In one embodiment the nucleotides of the first binding domain comprise at least one modified internucleoside bond. In another embodiment the nucleotides of the second binding domain comprise at least one modified internucleoside bond. In a further embodiment the modified internucleoside bond is a phosphorothioate bond.

The nucleic acid may comprise modified bonds. For example the bonds between nucleotides of the catalytic nucleic acid may comprise phosphorothioate linkages. The nucleic acid may comprise nucleotides having moiety may be modified by replacing one or both of the two bridging oxygen atoms of the linkage with analogues such as-NH, —CH2, or —S. Other oxygen analogues known in the art may also be used. The phosphorothioate bonds may be stereo regular or stereo random.

Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

This invention also provides a vector which comprises a sequence encoding any of the instant catalytic nucleic acid molecules. This invention also provides a host-vector system comprising a cell having the instant vector therein.

This invention also provides a method of producing the instant catalytic nucleic acid molecules comprising culturing a cell having therein a vector comprising a sequence encoding said catalytic nucleic acid molecule under conditions permitting the expression of the catalytic nucleic acid molecule by the cell.

This invention also provides a nucleic acid molecule that specifically hybridizes to an mRNA encoding Desmoglein 4 so as to inhibit the translation thereof in a cell. In one embodiment the nucleic acid is a ribonucleic acid. In one embodiment the nucleic acid is deoxyribonucleic acid. In one embodiment the nucleic acid molecule hybridizes to a site within the Hairless Protein mRNA located within the first 3000 residues following the mRNA's 5′terminus. In one embodiment the nucleic acid molecule hybridizes to a site within the mRNA encoding Desmoglein 4 located within the first 1500 residues following the mRNA's 5′ terminus. In one embodiment the nucleic acid molecule the mRNA encoding Desmoglein 4 is from a subject selected from the group consisting of human, monkey, rat and mouse.

This invention also provides a vector which comprises a sequence encoding the instant nucleic acid molecule. This invention also provides host-vector system comprising a cell having the instant vector therein.

This invention also provides a pharmaceutical composition comprising (a) the instant nucleic acid molecule or the instant vector and (b) a pharmaceutically acceptable carrier. In one embodiment the carrier is an alcohol. In one embodiment the carrier is ethylene glycol. In one embodiment the carrier is a liposome.

This invention provides a method of specifically inhibiting the expression of Desmoglein 4 in a cell that would otherwise express Desmoglein 4, comprising contacting the cell with the instant nucleic acid molecule so as to specifically inhibit the expression of Desmoglein 4 in the cell.

This invention provides a method of specifically inhibiting the expression of Desmoglein 4 in a subject's cells comprising administering to the subject an amount of the instant nucleic acid molecule effective to specifically inhibit the expression of Desmoglein 4 in the subject's cells.

This invention provides a method of specifically inhibiting the expression of Desmoglein 4 in a subject's cells comprising administering to the subject an amount of the instant pharmaceutical composition effective to specifically inhibit the expression of Desmoglein 4 in the subject's cells.

This invention provides a method of inhibiting hair production by a hair-producing cell comprising contacting the cell with an effective amount of the instant nucleic acid molecule.

This invention provides a method of inhibiting hair growth in a subject comprising administering to the subject an effective amount of the instant pharmaceutical composition.

In one embodiment of the instant methods the cell is a keratinocyte. In one embodiment of the instant methods the subject is a human. In one embodiment of the instant methods the nucleic acid molecule is administered topically. In one embodiment of the instant methods the nucleic acid is administered dermally.

This invention provides a method of producing the instant nucleic acid molecule comprising culturing a cell having therein a vector comprising a sequence encoding said nucleic acid molecule under conditions permitting the expression of the nucleic acid molecule by the cell.

This invention provides a non-human transgenic mammal, wherein the mammal's genome:

(a) has stably integrated therein a nucleotide sequence encoding a human Desmoglein 4 operably linked to a promoter, whereby the nucleotide sequence is expressed; and

(b) lacks an expressible endogenous Desmoglein 4 encoding nucleic acid sequence.

This invention provides a oligonucleotide comprising consecutive nucleotides that hybridizes with a Desmoglein 4-encoding mRNA under conditions of high stringency and is between 8 and 40 nucleotides in length. In one embodiment the oligonucleotide inhibits translation of the Desmoglein 4-encoding mRNA. In one embodiment least one internucleoside linkage within the oligonucleotide comprises a phosphorothioate linkage. In one embodiment the nucleotides comprise at least one deoxyribonucleotide. In one embodiment the nucleotides comprise at least one ribonucleotide. In one emboidment the Desmoglein 4-encoding mRNA encodes human Desmoglein 4. In one emboidment the Desmoglein 4-encoding mRNA comprises consecutive nucleotides, the sequence of which is set forth in SEQ ID NO: 2 or 4.

This invention provides a pharmaceutical composition comprising (a) the instant oligonucleotide and (b) a pharmaceutically acceptable carrier.

This invention provides a method of treating a subject which comprises administering to the subject an amount of the instant oligonucleotide effective to inhibit expression of a Desmoglein 4 in the subject so as to thereby treat the subject.

This invention provides a method of specifically inhibiting the expression of Desmoglein 4 in a cell that would otherwise express Desmoglein 4, comprising contacting the cell with the instant oligonucleotide so as to specifically inhibit the expression of Desmoglein 4 in the cell.

This invention provides a method of specifically inhibiting the expression of Desmoglein 4 in a subject's cells comprising administering to the subject an amount of the instant oligonucleotide effective to specifically inhibit the expression of Desmoglein 4 in the subject's cells.

This invention provides a method of specifically inhibiting the expression of Desmoglein 4 in a subject's cells comprising administering to the subject an amount of the instant pharmaceutical composition effective to specifically inhibit the expression of Desmoglein 4 in the subject's cells.

This invention provides a method of inhibiting hair production by a hair-producing cell comprising contacting the cell with an effective amount of the instant oligonucleotide.

This invention provides a method of inhibiting hair growth in a subject comprising administering to the subject an effective amount of the instant pharmaceutical composition. In one embodiment the subject is a mammal. In one embodiment the mammal is a human being.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, catalytic nucleic acids or antisense oligonucleotides may be formulated in accordance with this invention which are targeted wholly or in part to these associated ribonucleotides as well as to the informational ribonucleotides. For example, the antisense oligonucleotides may therefore be specifically hybridizable with a transcription initiation site region, a translation initiation codon region, a 5′ cap region, an intron/exon junction, coding sequences, a translation termination codon region or sequences in the 5′- or 3′-untranslated region. Similarly, the catalytic nucleic acids may specifically cleave a transcription initiation site region, a translation initiation codon region, a 5′ cap region, an intron/exon junction, coding sequences, a translation termination codon region or sequences in the 5′- or 3′-untranslated region. As is known in the art, the translation initiation codon is typically 5′-AUG (in transcribed mRNA molecules; 5′-ATG in the corresponding DNA molecule). A minority of genes have a translation initiation codon having the RNA sequence 5′-GUG, 5′-UUG or 5′-CUG, and 5′-AUA, 5′-ACG and 5′-CUG have been shown to function in vivo. Thus, the term “translation initiation codon” can encompass many codon sequences, even though the initiator amino acid in each instance is typically methionine in eukaryotes.

It is also known in the art that eukaryotic genes may have two or more alternative translation initiation codons, any one of which may be preferentially utilized for translation initiation in a particular cell type or tissue, or under a particular set of conditions. In the context of the invention, “translation initiation codon” refers to the codon or codons that are used in vivo to initiate translation of an mRNA molecule transcribed from a gene encoding PAI-1, regardless of the sequence (s) of such codons. It is also known in the art that a translation termination codon of a gene may have one of three sequences, i.e., 5′-UAA, 5′-UAG and 5′-UGA (the corresponding DNA sequences are 5′-TAA, 5′-TAG and 5′-TGA, respectively). The term “translation initiation codon region” refers to a portion of such an mRNA or gene that encompasses from about 25 to about 50 contiguous nucleotides in either direction (i.e., 5′ or 3′) from a translation initiation codon. This region is one preferred target region.

Similarly, the term “translation termination codon region” refers to a portion of such an mRNA or gene that encompasses from about 25 to about 50 contiguous nucleotides in either direction (i.e., 5′ or 3′) from a translation termination codon. This region is also one preferred target region. The open reading frame or “coding region,” which is known in the art to refer to the region between the translation initiation codon and the translation termination codon, is also a region which may be targeted effectively. Other preferred target regions include the 5′ untranslated region (5′ UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), known in the art to refer to the portion of an mRNA in the 3′ direction from the translation termination codon, and thus including nucleotides between the translation termination codon and 3′ end of an mRNA or corresponding nucleotides on the gene. mRNA splice sites may also be preferred target regions, and are particularly useful in situations where aberrant splicing is implicated in disease, or where an overproduction of a particular mRNA splice product is implicated in disease. Aberrant fusion junctions due to rearrangements or deletions may also be preferred targets.

Antisense oligonucleotides are chosen which are sufficiently complementary to the target, i.e., hybridize sufficiently well and with sufficient specificity, to give the desired disruption of the function of the molecule. “Hybridization,” in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the antisense oligonucleotide. Similarly, catalytic nucleic acids are synthesized once cleavage target sites on the Desmoglein 4-encoding mRNA molecule have been identified, e.g., any purine:pyrimidine consensus sequences in the case of DNA enzymes.

Methods for selecting which particular antisense oligonucleotides sequences directed towards a particular protein-encoding mRNA are that will form the most stable DNA: RNA hybrids within the given target mRNA sequence are known in the art and are exemplified in U.S. Pat. No. 6,183,966 which is herein incorporated by reference.

In one embodiment at least one internucleoside linkage within the instant oligonucleotide comprises a phosphorothioate linkage. Antisense oligonucleotide molecules synthesized with a phosphorothioate backbone have proven particularly resistant to exonuclease damage compared to standard deoxyribonucleic acids, and so they are used in preference. A phosphorothioate antisense oligonucleotide for Desmoglein 4-encoding mRNA can be synthesized on an Applied Biosystems (Foster City, Calif.) model 380B DNA synthesizer by standard methods. For example, sulfurization can be performed using tetraethylthiuram disulfide/acetonitrile. Following cleavage from controlled pore glass support, oligodeoxynucleotides can be base deblocked in ammonium hydroxide at 60° C. for 8 h and purified by reversed-phase HPLC [0.1M triethylammonium bicarbonate/acetonitrile; PRP-1 support]. Oligomers can be detritylated in 3% acetic acid and precipitated with 2% lithiumperchlorate/acetone, dissolved in sterile water and reprecipitated as the sodium salt from 1 M NaCl/ethanol. Concentrations of the full length species can be determined by UV spectroscopy. Any other means for such synthesis known in the art may additionally or alternatively be employed. It is well known to use similar techniques to prepare oligonucleotides such as the phosphorothioates and alkylated derivatives.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage, i.e., a single inverted nucleoside residue which may be abasic (the nucleobase is missing or has a hydroxyl group in place thereof). Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Determining the effective amount of the instant pharmaceutical composition can be done based on animal data using routine computational methods. In one embodiment, the effective amount contains between about 10 ng and about 100 μg of the instant nucleic acid molecules per square centimeter of skin. In another embodiment, the effective amount contains between about 100 ng and about 10 μg of the nucleic acid molecules per square centimeter of skin. In a further embodiment, the effective amount contains between about 1 μg and about 5 μg, and preferably about 2 μg, of the nucleic acid molecules per square centimeter of skin.

This invention further provides a host-vector system comprising a cell having the instant vector therein. This invention still further provides a method of producing either of the instant catalytic nucleic acid molecules comprising culturing a cell having therein a vector comprising a sequence encoding either catalytic nucleic acid molecule under conditions permitting the expression of the catalytic nucleic acid molecule by the cell. Methods of culturing cells in order to permit expression and conditions permitting expression are well known in the art. For example see Sambrook et al. (1989). Such methods can optionally comprise a further step of recovering the nucleic acid product.

Desmoglein 4 expression can also be inhibited using RNAi, as detailed in U.S. Pat. No. 6,506,599, the contents of which are hereby incorporated by reference.

In this invention, the various embodiments of subjects, pharmaceutically acceptable carriers, dosages, cell types, routes of administration and target nucleic acid sequences are envisioned for each of the instant nucleic acid molecules, pharmaceutical compositions and methods. Moreover, in this invention, the various embodiments of methods, subjects, pharmaceutically acceptable carriers, dosages, cell types, routes of administration and target nucleic acid sequences are envisioned for all non-nucleic acid agents which inhibit the expression of Hairless Protein. Such non-nucleic acid agents include, without limitation, polypeptides, carbohydrates and small organic compounds.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

Localized Hypotrichosis is Linked to Chromosome 18

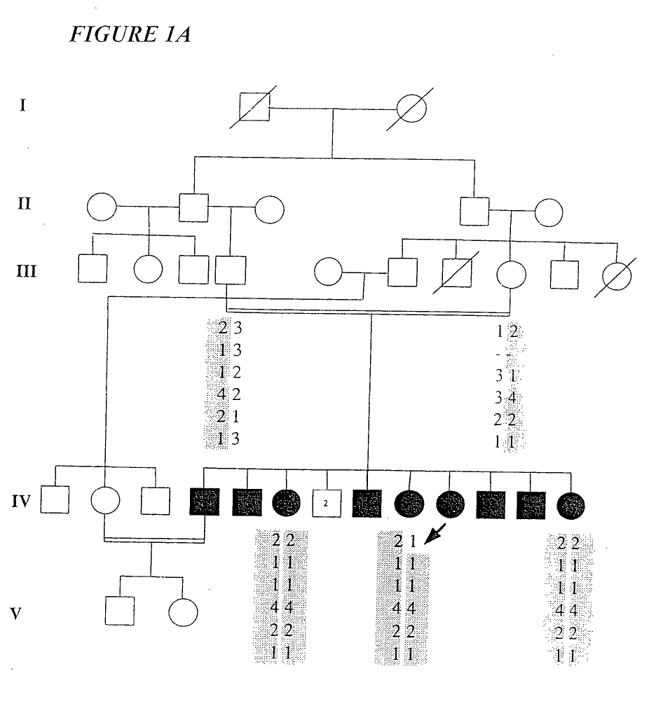

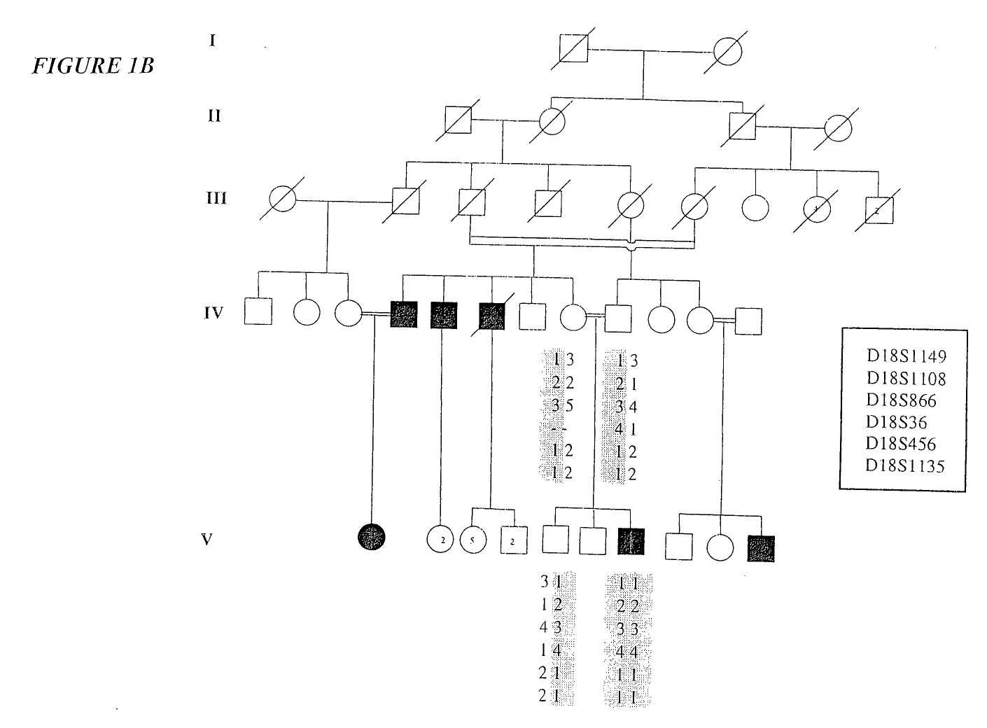

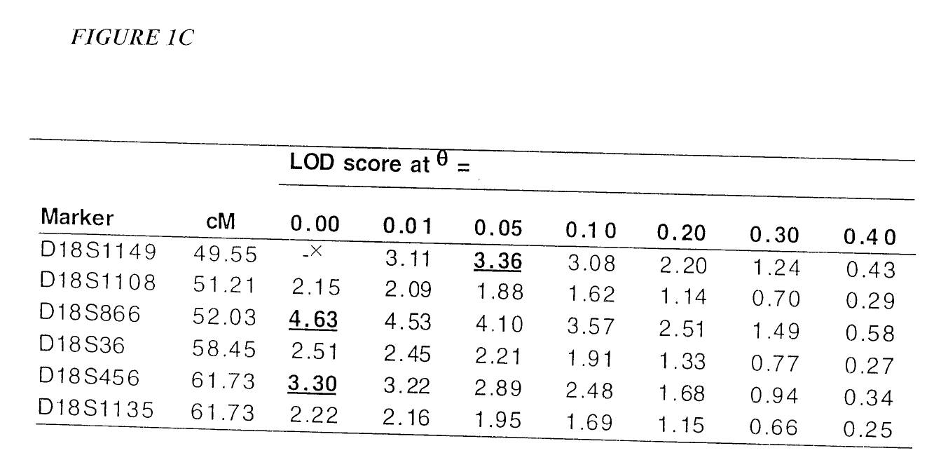

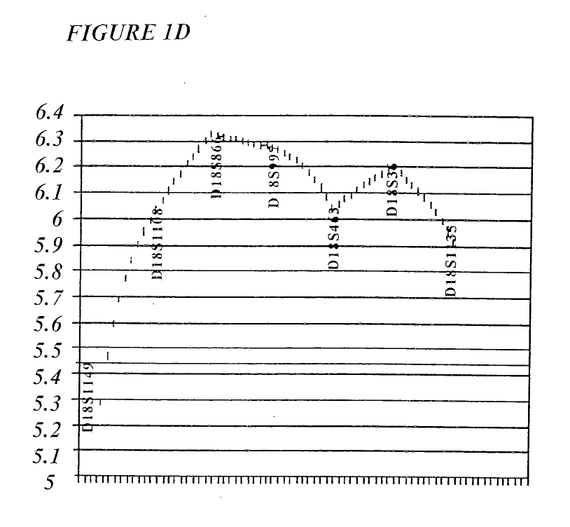

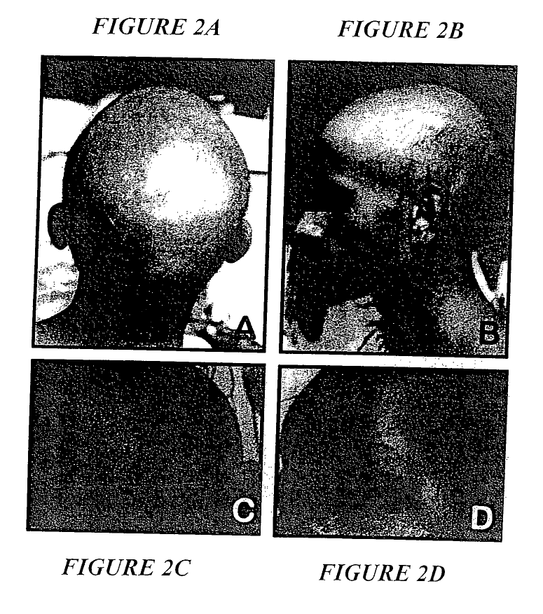

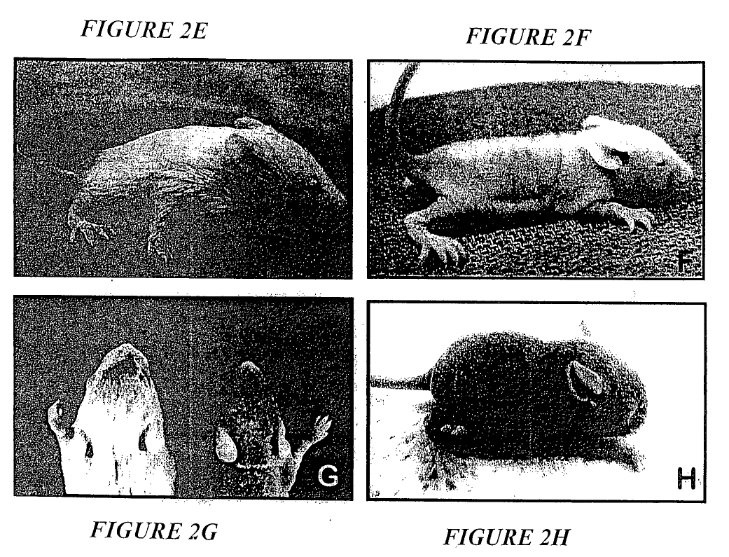

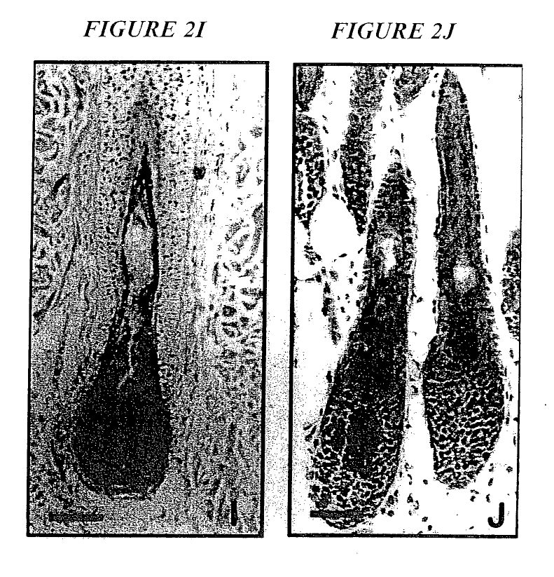

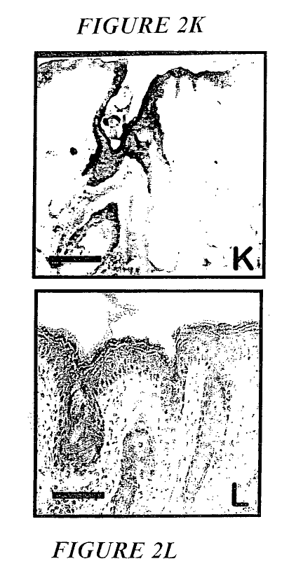

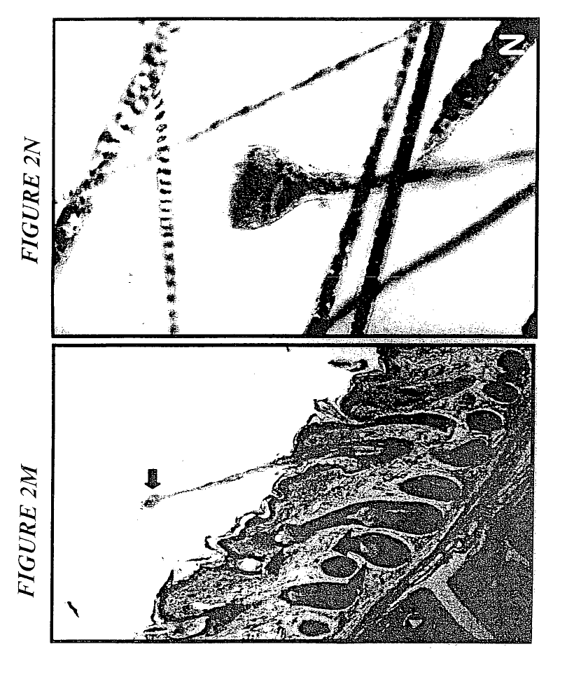

Two consanguinous Pakistani pedigrees with localized autosomal recessive hypotrichosis (LAH) were collected (FIG. 1A, B) in which affected members display hypotrichosis (FIG. 2A-D) restricted to the scalp, chest, arms, and legs. Facial hair, including the eyebrows and beard, is less dense, and axillary, pubic hair, and eyelashes are spared. Overall, the patients′ skin is normal with the exception of patches of scalp where small papules are visible that are likely a consequence of ingrown hairs. Histological analysis of scalp skin reveals abnormal HF and hair shafts (FIG. 21, K) that are thin and atrophic and often appeared coiled up within the skin due to their inability to penetrate the epidermis (FIG. 2K). Another striking defect is a marked swelling of the precortical region resulting in the formation of a bulbous “bleb” within the base of the hair shaft (FIG. 21).

To identify the gene underlying the LAH phenotype, we followed a classical linkage analysis approach. Prior to embarking on a genome-wide scan, we performed cosegregation and homozygosity analysis with microsatellite markers corresponding to candidate genes involved in related phenotypes. These included the desmosomal cadherin gene cluster on 18q12, the hairless gene on 8p21, the nude gene on 17g11, and the keratin clusters on chromosomes 12 and 17. Linkage was excluded for all regions, with the exception of the desmosomal cadherin gene cluster on chromosome 18. A maximum two-point LOD score (Zmax) of 4.63 was obtained for marker D18S866 (q=0), combining the LOD score values from the two pedigrees (FIG. 1C). Multipoint analysis supported linkage to this region, with maximum LOD scores exceeding 5.0 throughout the interval D18S1108-D18S1135 (FIG. 1D).

A key recombination event in individual IV-10 from pedigree LAH-1 (FIG. 1A), placed the LAH locus telomeric to D18S1149. Haplotype analysis using chromosome 18 markers (FIG. 1A, B) revealed that affected individuals were homozygous for all markers in the interval between D18S1149 and D18S1135, and shared an identical haplotype for D18S36. According to the physical map from the Human Genome Project Working Draft (April 2002 release), D18S36 lies 0.5 Mb centromeric to the desmosomal cadherin gene cluster (Buxton et al., 1993). All exons and splice sites from the six genes were sequenced in affected members from both families, however, no mutations were identified.

Comparative Genomics Reveals Synteny with the Lanceolate Hair Phenotype

The LAH syntenic region on mouse Chromosome 18 contains the locus for an autosomal recessive mutation, lanceolate hair (lah), and also harbors the desmosomal cadherin cluster (Montagutelli et al., 1996). lah/lah pups develop only a few short, fragile hairs on the head and neck which disappear within a few months. The vibrissae are short and abnormal and the pups have thickened skin. Mutant lah/lah mice do not exhibit any growth retardation relative to their unaffected littermates (FIG. 2E, F). A second allele of lanceolate hair, named lahJ, later arose as a spontaneous mutation at the Jackson Laboratories (FIG. 2G, H), and complementation established that the two mutations were allelic (Sundberg et al., 2000). The lahJ/lahJ phenotype is more severe, as the pups fail to grow any normal hairs and completely lack vibrissae. Instead, the pups are covered with abnormally keratinized stubble giving the mouse a “peach fuzz” appearance (Sundberg et al., 2000). Histological analysis of HFs in both lah/lah and lahJ/lahJ reveals striking similarities to human LAH (FIG. 21-L). The main feature is the formation of a swelling above the melanogenic zone. The ‘bleb’ is then pushed up with the progression of the hair growth, leaving the distal end of the hair shaft with a lance-head shape, hence the name lanceolate hair. Occasionally, two blebs can be observed within a single anagen follicle (FIG. 2M). Degenerative changes in the hair shaft include the loss of the ladder-like pattern of pigment distribution in the medulla, which is replaced by chaotically distributed amorphous pigment granules and air spaces (FIG. 2N). In contrast to human LAH patients, the interfollicular epidermis in both mouse lanceolate alleles is significantly thickened exhibiting prominent hyperplasia (FIG. 2L, M).

Genetic mapping had previously placed the lah mutation in the syntenic region of mouse Chromosome 18. Mutations in the Dsg3 gene underlie the balding phenotype, and complementation matings indicated that bal/bal and lah/lah are not allelic (Montagutelli et al., 1996). We screened the remaining desmosomal cadherin genes, and detected no mutations or differences in mRNA levels.

Desmoglein 4, a Member of the Cadherin Superfamily

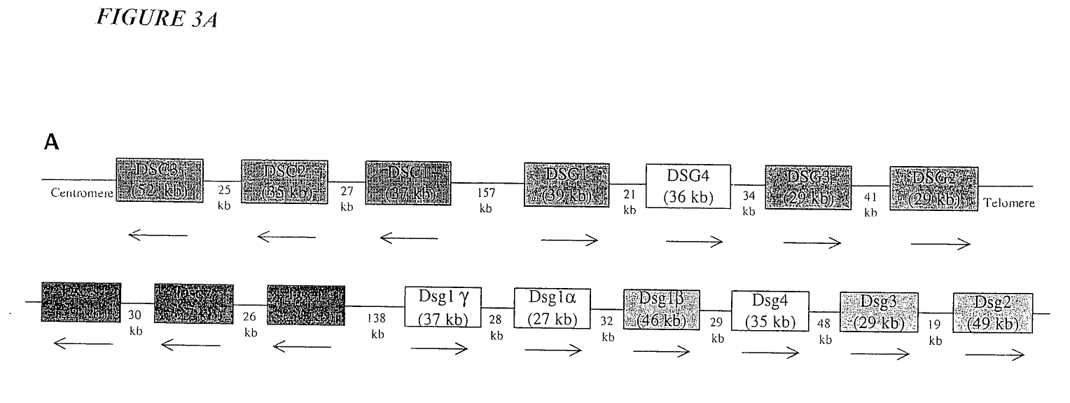

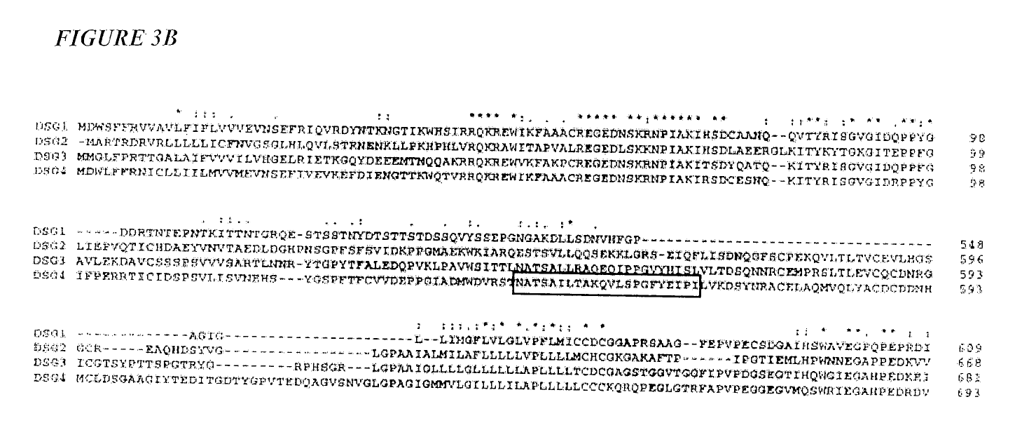

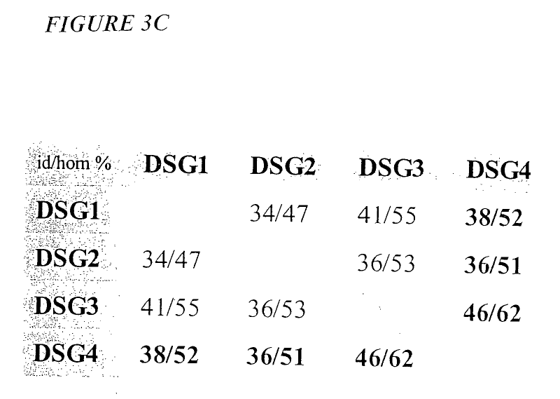

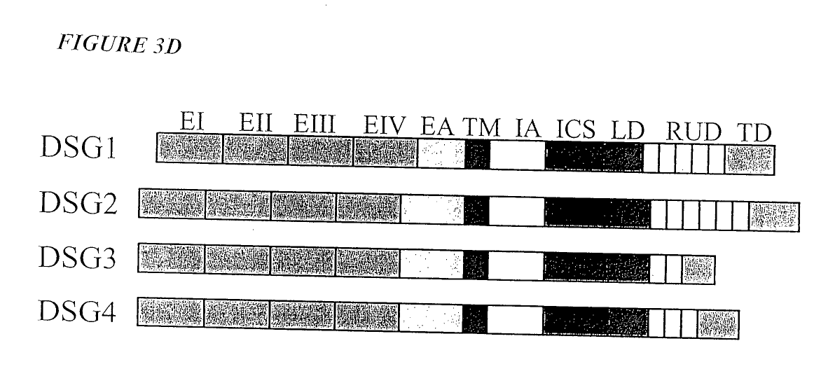

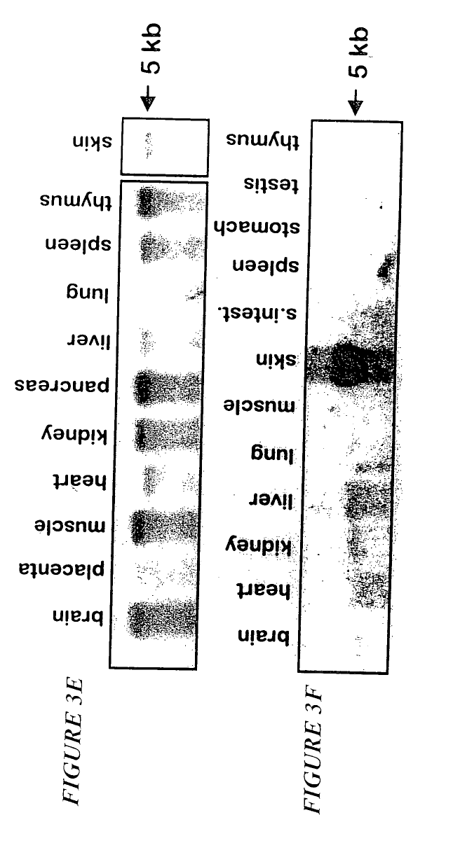

Unexpectedly, in the process of detailed genomic analysis in the mouse, we identified three previously undescribed cadherin genes within the cluster (FIG. 3A). Two of these, Dsglb and Dsglg, are not found in the human genome, and are reported elsewhere (Kljuic and Christiano, 2003; Pulkkinen et al., 2003). The third cadherin was also present in the human genome, and was designated desmoglein 4 in mouse (Dsg4) and human (DSG4) (FIG. 3A-D), which share 79% and 86% amino acid identity and homology, respectively. A comparison of the structural organization and homology analysis of DSG4 to the other desmogleins is depicted in FIGS. 3B-D. The human and mouse mRNA was highly expressed in skin (FIG. 3E, F), and together with its co-localization within the lanceolate and LAH linkage intervals, desmoglein 4 became a candidate gene for both phenotypes.

Dsg4 is Mutated in Human LAH and Lanceolate Mice

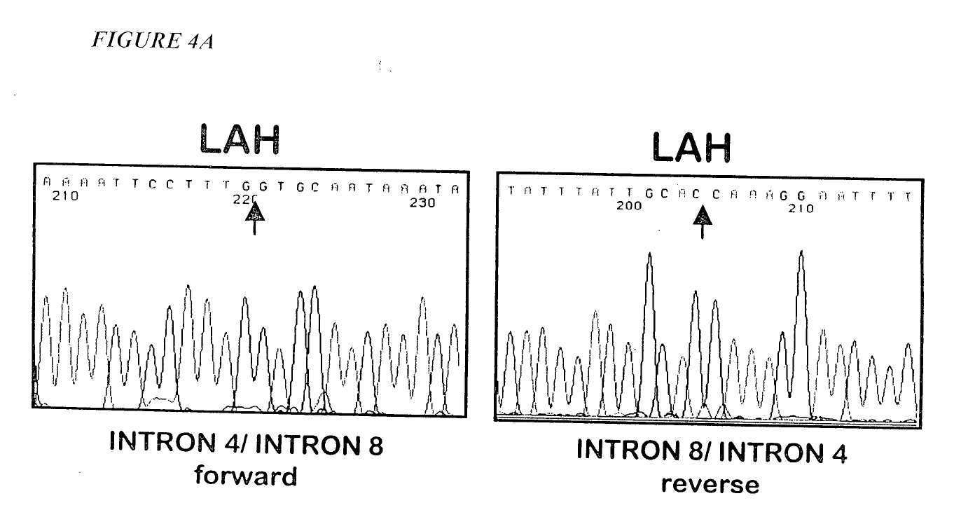

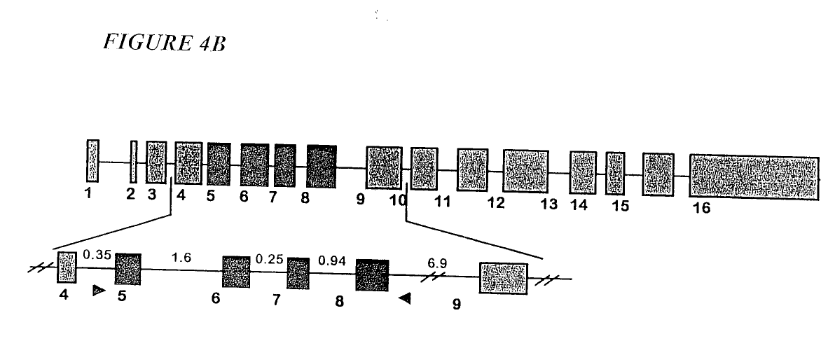

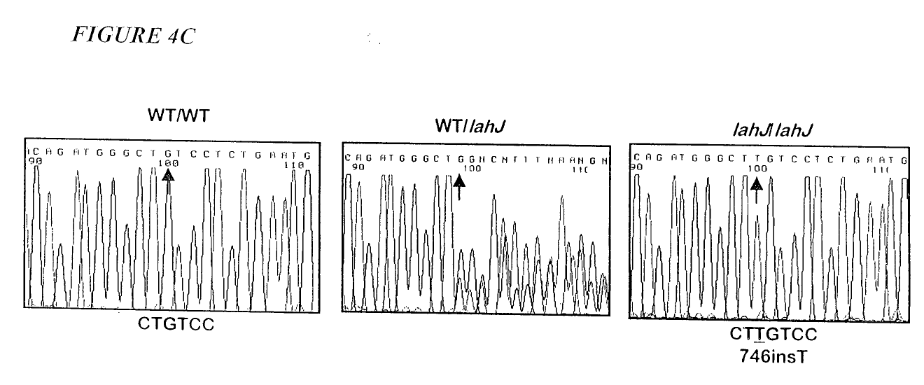

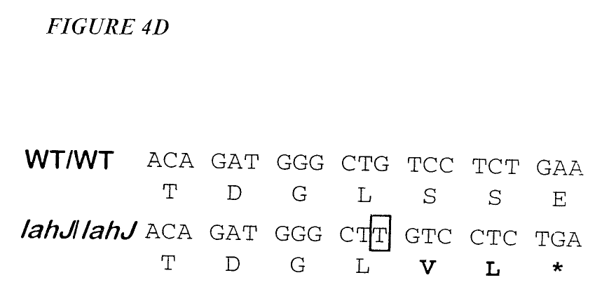

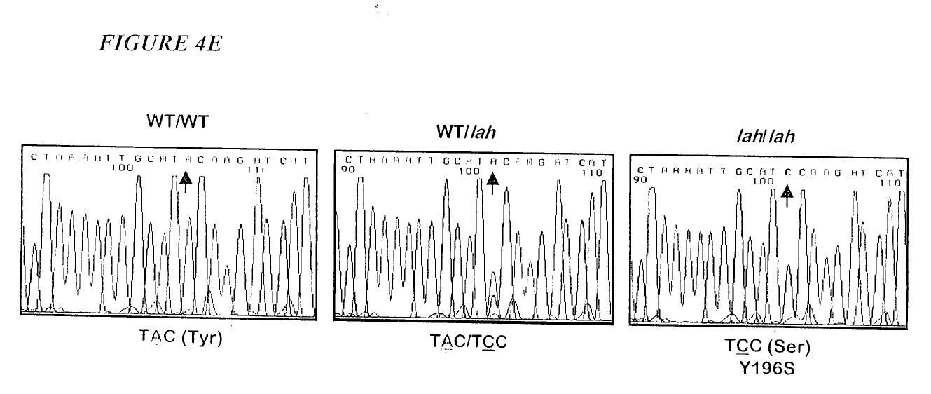

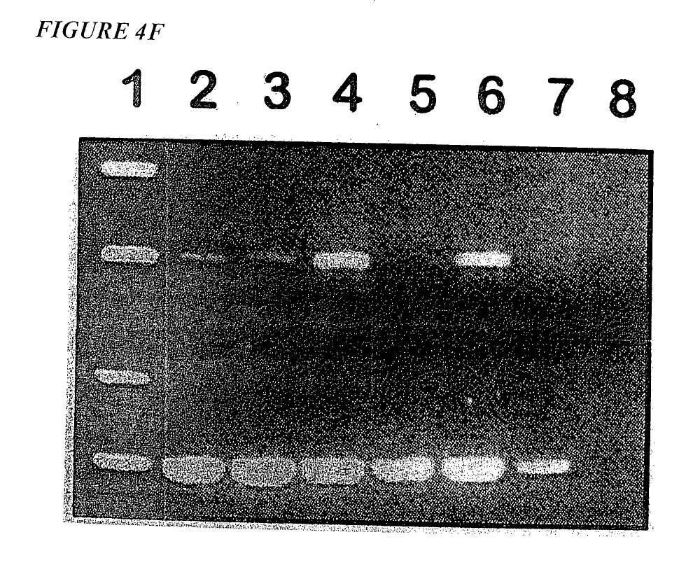

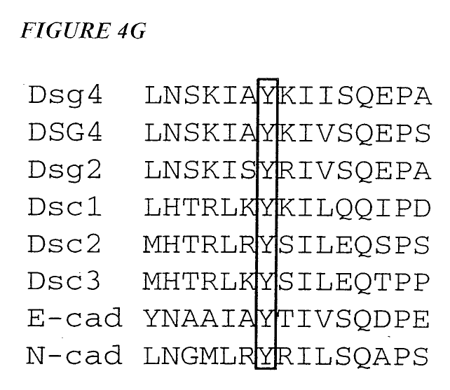

We identified an identical homozygous intragenic 5 kb deletion in affected individuals from both LAH families by direct sequencing (FIG. 4A, B). The deletion begins 35 nucleotides upstream of exon 5 and ends 289 nucleotides downstream of exon 8. This mutation, designated EX5_{—}8del, generates an in-frame deletion creating a predicted protein missing amino acids 125-335. Sequence analysis of Dsg4 in lahJ/lahJ animals revealed a single base insertion following nucleotide 746 within exon 7, designated 746insT (FIG. 4C). The frameshift creates a premature termination codon three codons downstream from the insertion (FIG. 4D). RT-PCR data show that the mutant mRNA undergoes nonsense mediated decay, as we were unable to detect any Dsg4 mRNA (FIG. 4F) (Frischmeyer and Dietz, 1999). Sequence-analysis of Dsg4 in lah/lah animals identified a homozygous A-to-C transversion at nucleotide 587. This mutation converted a tyrosine residue (TAC) in exon 6 to a serine residue (TCC), designated Y196S (FIG. 4E). Y196 is conserved in the majority of desmosomal cadherins, as well as classical cadherins such as E-and N-cadherin (Figure 4G) and protein prediction software suggested that it represents a potential phosphorylation site. Extensive BLAST searches and sequencing of additional mouse strains indicated that Y196S is not a polymorphism. Thus, the lahJ/lahJ mouse serves as a null mutant animal model, whereas the lah/lah mouse represents a hypomorph. The revised designation of the mouse mutations is Dsg41ah/Dsg41ah and Dsg4lahJ/Dsg4lahJ.

Dsg4 is the Principal Desmosomal Cadherin in the Hair Follicle

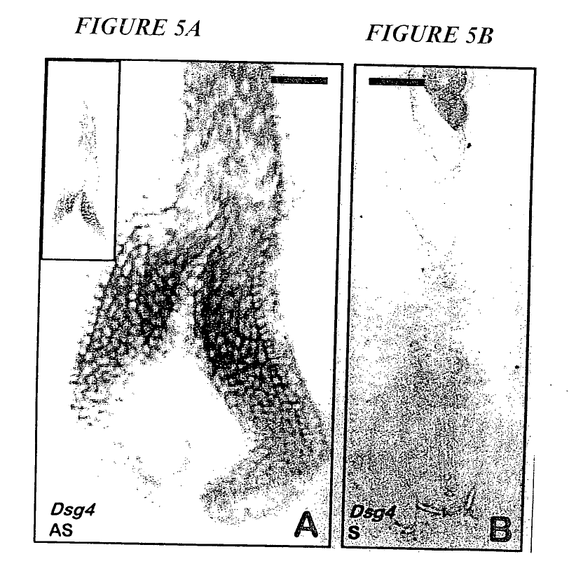

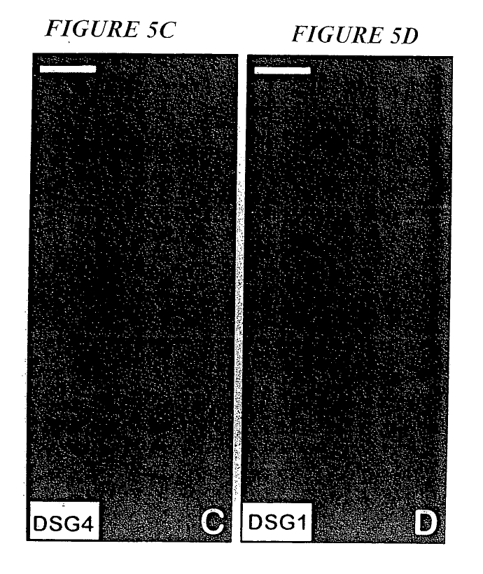

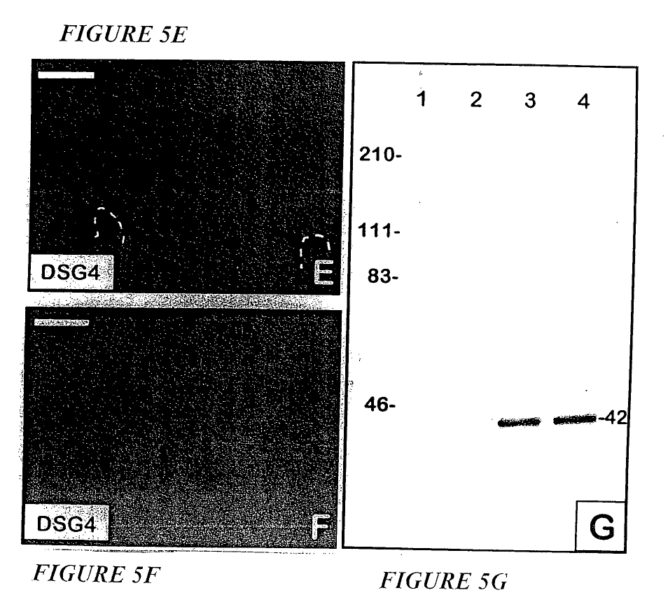

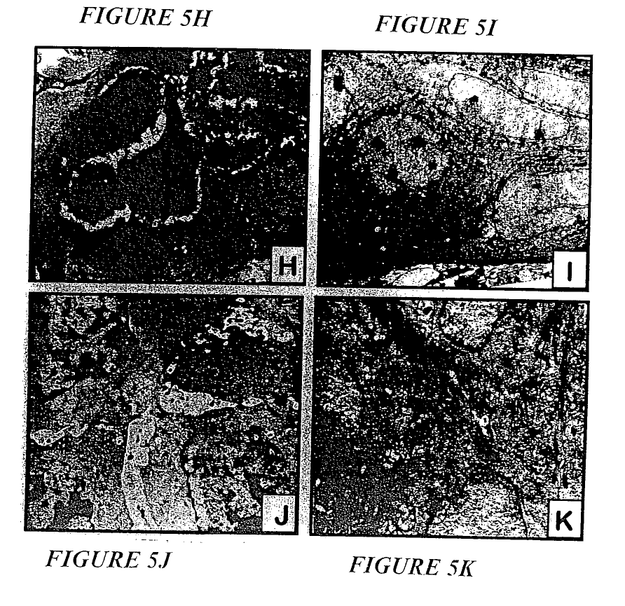

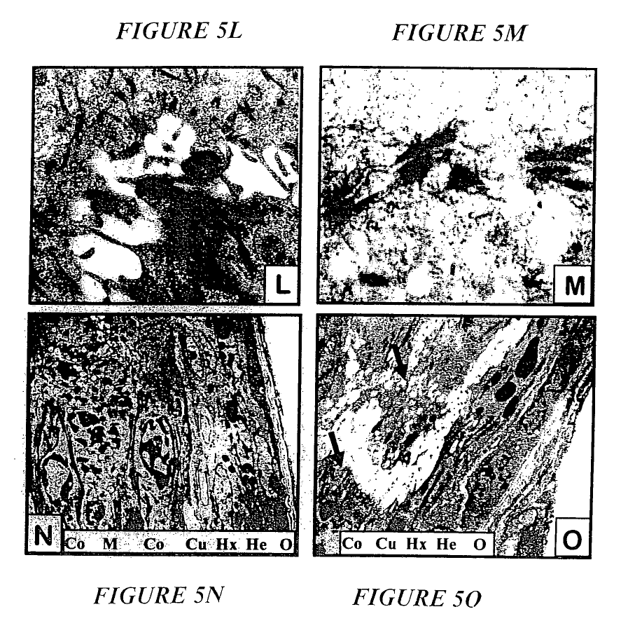

In situ hybridization of mouse skin sections and vibrissae follicles revealed that Dsg4 is expressed in anagen stage HFs. The mRNA was localized to the cells of the matrix, precortex and IRS of both pelage hair and vibrissae HF (FIG. 5A, B). DSG4 was also detected within anagen follicles where its expression commenced in the matrix and extended throughout precortical cells and IRS (FIG. 5C). The presence of desmoglein 4 in the inner layer of the HF, where DSG1 (FIG. 5D), DSG2, and DSG3 (Kurzen et al., 1998) are absent, suggests a critical role for desmoglein 4 in differentiation of the ascending HF layers.

Desmoglein 4 is Expressed in Suprabasal Epidermis and is a Target of PV Auto Antibodies

Immunofluorescent labeling of human scalp sections with DSG4 antibody revealed cell border localization of the protein within the suprabasal layers of the epidermis, where it is highly expressed (FIG. 5E, F). To test the hypothesis that DSG4 could serve as an autoantibody in PV similar to DSG1 and 3, we reacted sera of two PV patients with active skin and oral lesions against a recombinant N-terminal protein of DSG4, demonstrating that DSG4 is also an autoantigen in PV (FIG. 5G).

Desmosomes are Defective in lahJ/lahJ Mutants

Transmission electron microscopy of day 14 epidermis and HF from lahJ/lahJ mutant pups established the central role of Dsg4 in cell-cell adhesion. At low magnification, acantholysis along cell-cell borders was evident in all layers of mutant epidermis (FIG. 5H,I). The junctions between adjacent keratinocytes in lahJ/lahJ revealed complete detachment in some areas, and small, poorly formed desmosomes in others, into which filaments were only scantily inserted (FIG. 5J, K). Spaces between detached mutant keratinocytes revealed areas in which desmosomes have been torn away from their cells (FIG. 5L,M). Ultrastructural defects in keratinization of the inner layers of the hair shaft were evident in mutant HFs, consisting of a disorganized-array of air spaces and pigment granules in the medulla (FIG. 5N), and the complete detachment of keratinocytes in Henle's and Huxley's layers and the cortex. The cells are severed from their neighbors, leaving behind a row of detached desmosomes (FIG. 5O).

lahJ/lahJ Keratinocytes Exhibit a Hyperproliferative Phenotype

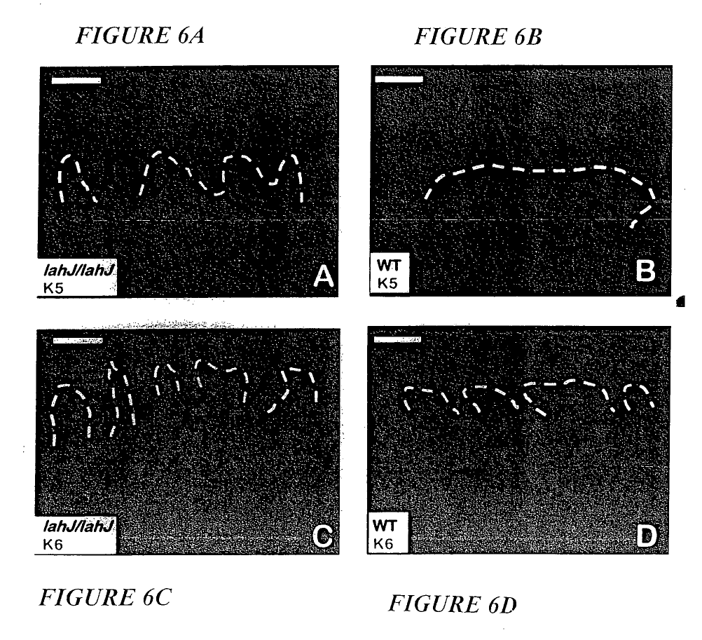

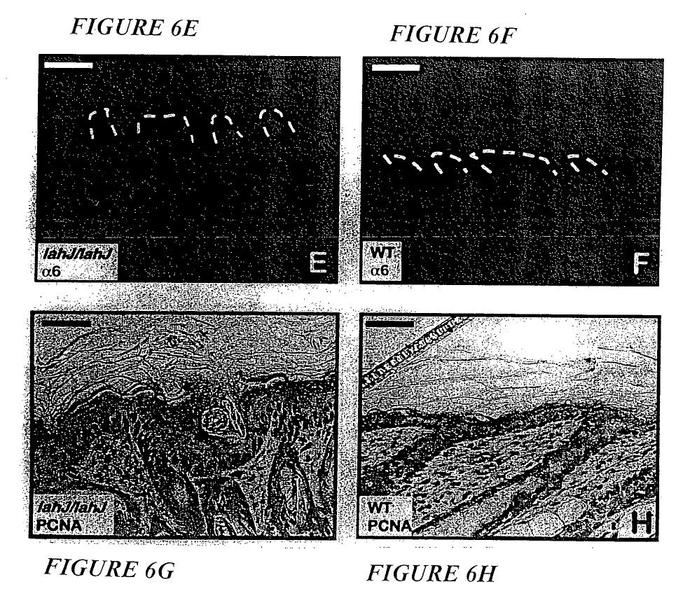

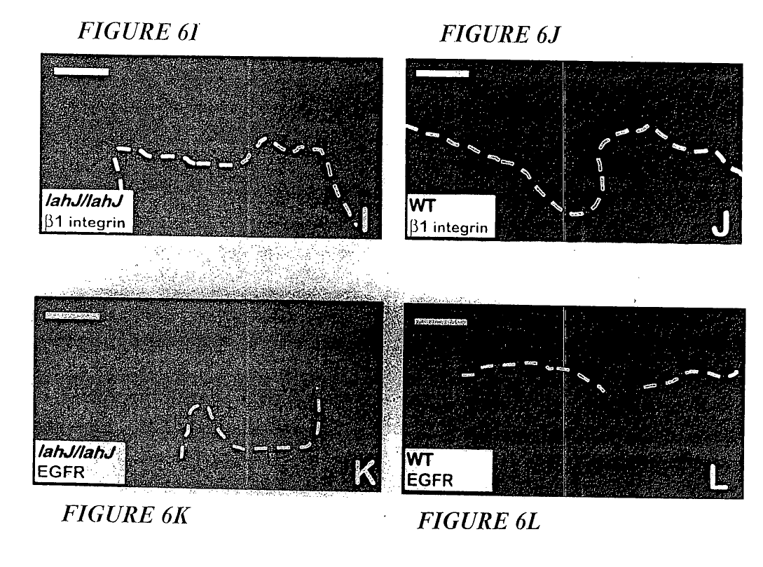

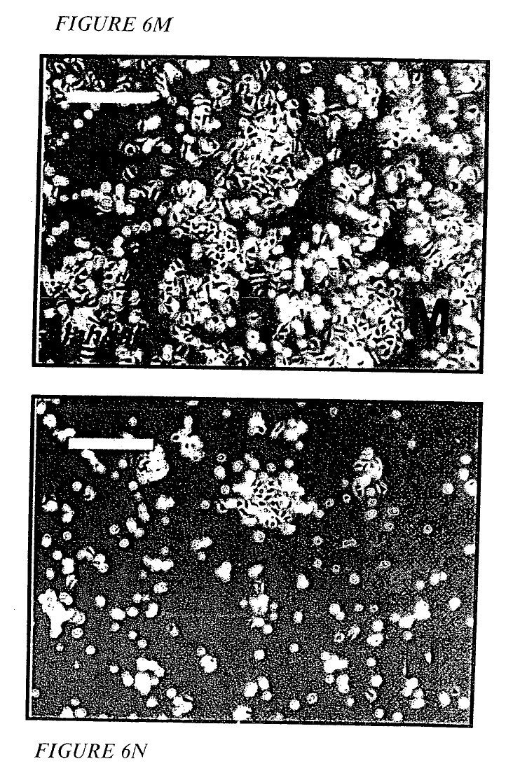

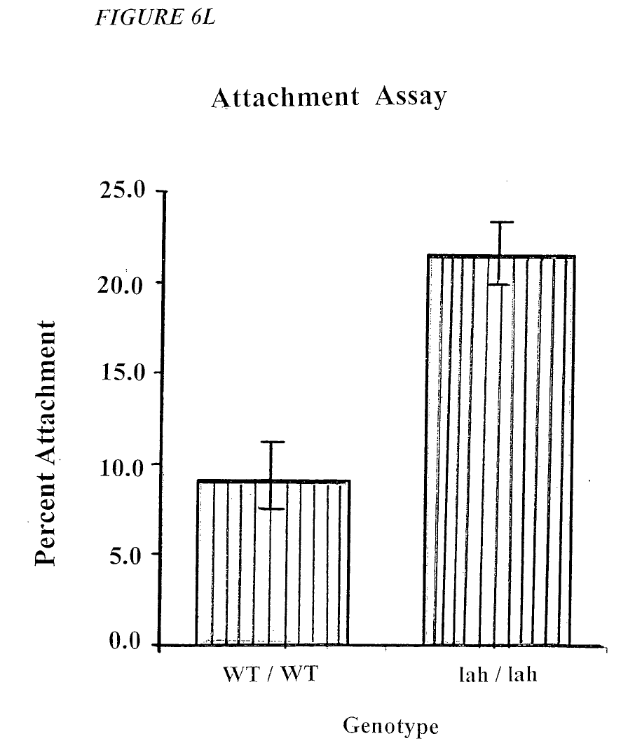

In order to further characterize the hyperplastic changes within the skin of mutant animals, we first assayed the expression of several epidermal markers. K5 was ubiquitously and evenly expressed in the basal layer of WT skin, compared to a patchy pattern of expression with fewer strongly positive basal cells in lahJ/lahJ mutants (FIG. 6A, B). The hyperproliferation marker K6 was significantly overexpressed in the spinous layer of the epidermis and HF infundibulum of mutant animals (FIG. 6C, D). The expression of a6 integrin, a hemidesmosomal marker, was markedly reduced in the basal layer of the interfollicular epidermis of the mutants (FIG. 6E, F). Expression of involucrin, loricrin, K1, Dsc1, 2,3, Dsg1, 3, b catenin, E-cad, P-cad, Pkpl, Dsp, Pg, were unchanged between WT and mutant animals (not shown).

In mutant epidermis, the finding of patchy K5 staining, the presence of K6 and the reduced expression of a6 integrin were all consistent with premature or accelerated exit of keratinocytes from the basal compartment. Consistent with the hypothesis that the proliferative compartment might therefore be expanded, we detected a higher number of PCNA expressing keratinocytes in the basal epidermis of lahJ/lahJ animals, as well as the existence of ectopically proliferating cells in the suprabasal layers (FIG. 6G, H). To further investigate the nature of the hyperproliferative phenotype, we assayed the expression of S1 integrin and EGFR and found that both were ectopically expressed in the suprabasal layers of mutant epidermis (FIG. 6I-L), while we found no difference in the expression pattern of total or activated MAP kinase (not shown). TUNEL staining was performed to assess the extent of apoptosis in mutant epidermis and HF, and no differences were detected compared to control animals (not shown).

Cell attachment kinetics of primary epidermal keratinocytes were performed to further characterize the skin of lah/lah mice. Attachment assays showed greater than two-fold enhanced attachment of lah/lah keratinocytes (21.7±1.8% of total seeded cells), compared to WT (9.0±2.1%) after 24 hrs in culture on vitrogen-fibronectin coated dishes (FIGS. 6M-6O). In this respect, it is noteworthy that lah/lah keratinocytes were also able to attach to uncoated plastic dishes, while the WT keratinocytes failed to do so. lah/lah keratinocytes formed fully confluent monolayers by day 4 of culture in low Ca⁺⁺, whereas the WT keratinocytes reached only 60-70% confluency during the same period, suggesting an enhanced ability of lah/lah cells to spread, explaining why they precociously form monolayers in culture. Since epithelial sheets do not form in low Ca⁺⁺ conditions, we compared the response of lah/lah and WT keratinocytes when both are induced to differentiate in high Ca++ medium. Upon switching to high Ca⁺⁺ conditions, the mutant keratinocytes behaved similar to WT cells and no morphological differences were seen for up to 3 weeks. We assayed the expression levels and assembly status of intermediate filament and adhesion components in primary cultured keratinocytes, and found no differences in K5, Dsgl, Pg, Pkpl or actin (not shown).

lahJ/lahJ Hair Matrix Keratinocytes Exhibit Disrupted Differentiation

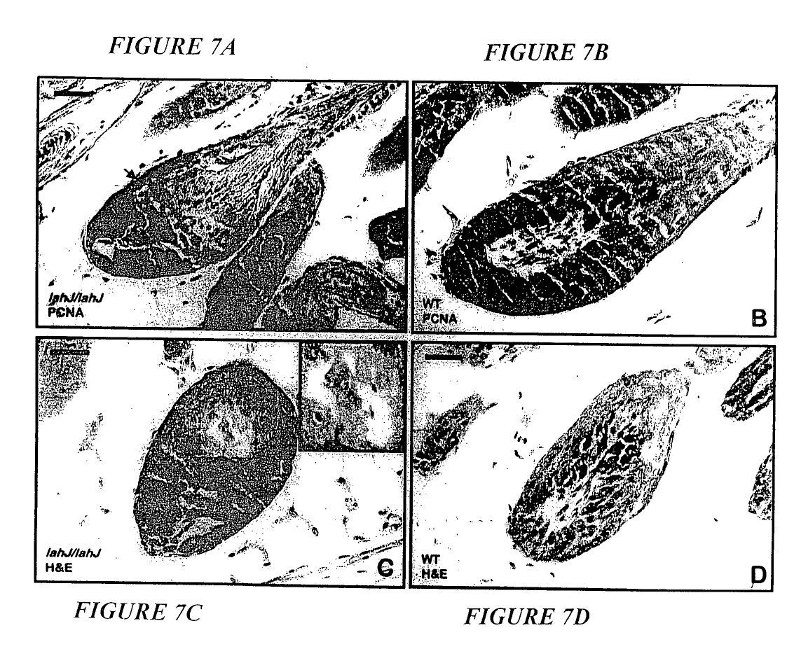

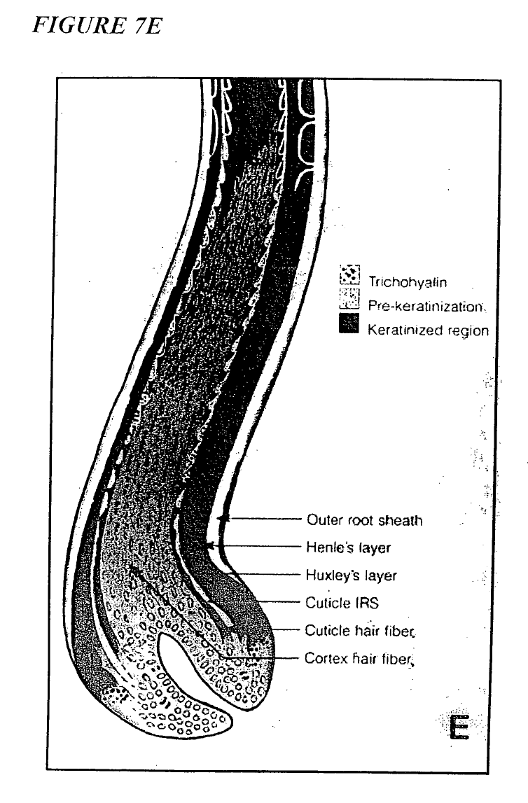

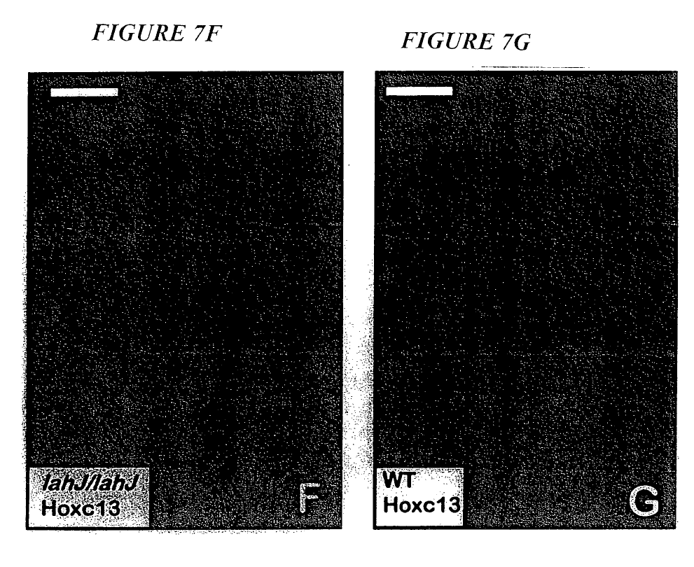

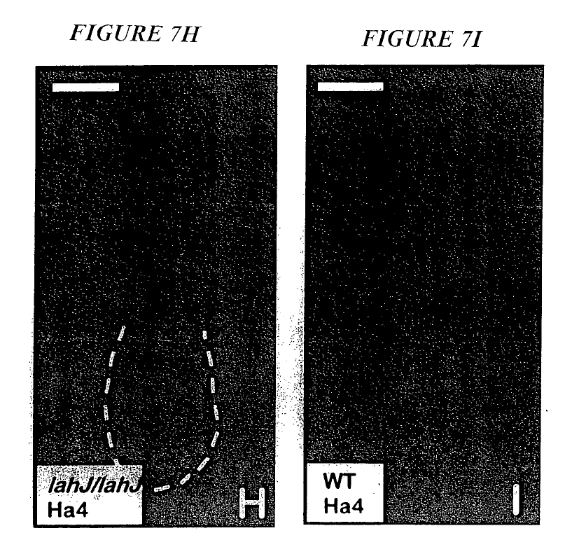

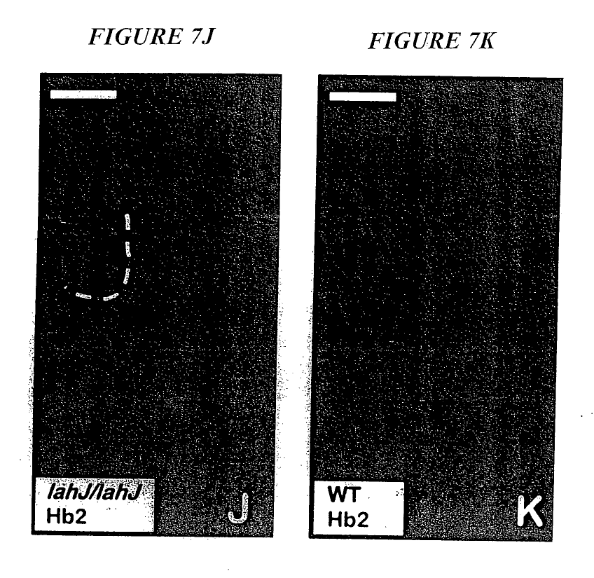

The transition from proliferation to differentiation in the lower HF occurs along a gradient as cells pass through the line of Auber. In WT matrix keratinocytes, we observed the expected graduation from the base of the follicles, where all cells are proliferating, to the precortex, where essentially all cells are differentiating (FIG. 7A, B). Strikingly, in mutant HF we instead observed a dramatic cessation of proliferation and an abrupt transition to differentiation between adjacent cells (FIG. 7A, B). The premature loss of the proliferative signal and sudden switch to differentiation occurs precisely in the region of cell-cell separation (FIG. 7C, D) and the onset of the formation of the lance head.

We then assessed the expression of hoxC13 and the hair keratins hHb2 and hHa4, which are specific for hair shaft cuticle and cortex differentiation, respectively. While both proteins are expressed in mutant follicles, their expression is spatially restricted compared to WT follicles. In WT follicles, both proteins are expressed in the upper bulb and in the middle portion of the HF, whereas in mutant HF they are restricted to a much smaller zone at the bulb narrowing (FIGS. 7F-I). HoxC13 regulates the expression of early hair keratins and is normally expressed in upper matrix/lower precortex, above the zone of hHa4 expression, as well as in the hair cuticle (FIG. 7J). In mutant skin, hoxC13 is significantly reduced in the lower hair follicle and is nearly undetectable in the cuticle (FIG. 7K).

Discussion

While many examples of correlations of human disorders with mouse models exist in the literature, there are very few which represent pure forms of alopecia without ectodermal dysplasia. We established the close correlation of the hairless mouse phenotype with atrichia with papular lesions (Ahmad et al., 1998) and the nude mouse phenotype with congenital alopecia and T-cell immunodeficiency (Frank et al., 1999), both of which result from defects in transcription factors. To our knowledge, there have been no reports to date of defects in structural proteins in mice that closely mimic a human hair disorder (Tong and Coulombe, 2003). LAH and lanceolate, therefore, represent corresponding human and mouse phenotypes resulting from defects in structural component of the epidermis and HF, desmoglein 4. The biological relevance of these findings extends into the area of skin autoimmunity, since we show that DSG4 also serves as an autoantigen in patients with PV (Nguyen et al., 2000). We have used both a naturally occurring null mutant (lahJ/lahJ) and hypomorphic (lah/lah) mouse model to begin dissecting the role of Dsg4 in epidermal and HF homeostasis and disease. Our findings demonstrate a central role of desmoglein 4 in keratinocyte cell adhesion, and furthermore, in coordinating cellular dynamics in the lower HF during the switch from proliferation to differentiation. Our findings further indicate that antisense ribozyme or other such inhibitory technologies can be directed to cause transient hairloss by inhibition of Desmoglein-4.

Dsg4 Is Critical for Intercellular Adhesion and Keratinocyte Differentiation

Our ultrastructural results suggest that desmoglein 4 participates in a desmosomal junction with a highly specialized function during hair shaft differentiation. The three-dimensional architecture of the HF itself imparts critical positional information to the cellular dynamics of hair growth, and as such, the maintenance of cell attachment is particularly critical during differentiation (Bullough and Laurence, 1958; Van Scott et al., 1963). The HF layers (FIG. 7E) are morphologically distinct, desmosome-rich, cylindrical epithelial sheets that keratinize in a temporally autonomous pattern during anagen, and are each characterized by a distinct signature of hair keratins. The rate of mitosis below the line of Auber must be precisely synchronized with the switch to differentiation, so that specific programs are executed at the correct time within a given layer (Auber, 1952). Further, as the differentiating cells of the precortex are forced upward through the narrow neck of the “funnel” created by the external HF membranes, they are under considerable mechanical pressure (Bullough and Laurence, 1958; Van Scott et al., 1963). We provide evidence that the requirement of HF keratinocytes to smoothly transition from proliferation to differentiation (FIG. 7A, B), to resist shear forces as they ascend (FIGS. 2,5, 7), and to differentiate along a different pathway than their neighbor (FIG. 7F-K) is critically dependent on cell-cell attachment mediated in part by desmoglein 4.

Absence of Dsg4 Leads to Epidermal Hyperproliferation

Our initial histological observations of mutant epidermis revealed marked thickening and hyperplasia, which prompted us to more closely examine the mechanism by which this occurred. Mutant epidermis revealed a profile of alterations consistent with an activated keratinocyte phenotype, specifically, downregulation of a6 integrin and K5 in the basal layer, suggesting a premature exit from the basal compartment. We detected marked upregulation of K6 throughout mutant epidermis (FIG. 6C), as well as a prominent increase in the number of PCNA-positive proliferating cells in the basal and suprabasal layers. We next asked whether this phenotype might be accompanied by the classical mediators of this phenomenon (Rikimaru et al., 1997), and found that both S1 integrin and EGFR were ectopically expressed in the suprabasal layers in mutant epidermis (FIG. 6H-K). In the context of lanceolate mutant animals, the triad of PCNA, S1 integrin and EGFR in the suprabasal cells correlates with defective cell adhesion in the epidermis. Additionally, the absence of nuclear MAPK in hyperproliferative epidermis suggests that in lah/lah mutants, EGFR may be signaling via an alternate pathway. Although the causes versus effects of suprabasal integrin expression are incompletely understood at present, the examples reported to date have been associated with an inflammatory response (Carroll et al., 1995). lah/lah mutant animals exhibit all the hallmarks of this response in the absence of inflammation, suggesting that the two events may be separable. Since K6 represents a transcriptional target of EGFR signaling (Jiang et al., 1993) and is strongly upregulated in mutant epidermis, it is likely that the hyperproliferative phenotype in lahJ/lahJ mutants is mediated by activation of additional EGF target genes.

The unexpected finding of several key hyperproliferative markers in the epidermis led us to more closely investigate the proliferative, properties of both epidermal and HF cells in lanceolate mutant animals. Quantitation of cellular kinetics revealed that lah/lah primary mouse keratinocytes exhibited enhanced cell spreading in addition to attachment, typical of activated or wound healing keratinocytes (Freedberg et al., 2001; Grinnell, 1990). One explanation for these findings is simply that in the absence of correct cell-attachment, the cells exhibit characteristics of activated keratinocytes. A similar mechanism was recently proposed for the enhanced attachment phenotype of keratinocytes from a patient with mutations in plectin, a hemidesmosomal component (Kurose et al., 2000). It is well-established that transient alterations of EGFR expression and activation are known to have profound effects on keratinocyte attachment, spreading and migration particularly during wound healing (Hudson and McCawley, 1998). Consistent with its overexpression in the epidermis, we hypothesize that the cell kinetic behavior of lah/lah mutant keratinocytes is also mediated by the activation of genes downstream of EGFR.

What makes a Lanceolate Hair?

The most striking aspect of the lanceolate phenotype is a transient, intermittent defect in differentiation of the HF precortical cells. Early in anagen, the growing follicles at first appear essentially normal, until some cells undergo a marked engorgement in the precortex region, resulting in a bleb within the hair shaft. In the center of the bleb, cells are torn away from their neighbors (FIG. 7C, D), and subsequently undergo premature, abnormal and rapid keratinization.

What is the mechanism by which absence of desmoglein 4 results in perturbed differentiation of HF keratinocytes? Emerging evidence suggests that the adhesive role of intercellular junctions, such as desmosomes, may in and of itself confer enhanced signaling by bringing apposing cell membranes into closer proximity, thereby facilitating other types of connections such as communicating junctions and ligand/receptor interactions (Jamora and Fuchs, 2002). Such interactions impact upon the diffusion of secreted factors across cell membranes and facilitate the establishment of morphogen gradients by positioning of their cognate transmembrane receptors. Importantly, cell adhesion molecules provide support for the extracellular matrix proteoglycans between cells that are required for transmission of signals such as Wnts and BMPs (Paine-Saunders et al., 2002).

One explanation for the origin of the lanceolate hair is that the abnormal precortical cells in lanceolate HF represent a population of naive keratinocytes that have been incompletely programmed upon their exit from the proliferation zone. We have shown by PCNA expression in mutant HF that the transition from proliferation to differentiation is dramatically disturbed, and that rather than proceeding along a gradient, instead it occurs abruptly (FIG. 7A, B). Given the complexity of signaling programs that are active in this region, including BMPs, Wnts and Notch/Delta, it is likely that the primary defect in cell adhesion also precipitates the inability of these signaling molecules to fully execute cell fate determination in this region. Evidence in support of this hypothesis includes perturbed expression of hoxC 13 and the cuticle and cortex hair keratins in mutant animals (FIG. 7), all three of which are downstream markers of both BMP and Wnt signaling in the HF precortex (Kulessa et al., 2000). The uncoupling of the transition from proliferation to differentiation further demonstrates that the transmission of survival signals is disrupted in the absence of intact cell-cell adhesion. What results then is a total communication breakdown in the lower HF, resulting in failed execution of differentiation programs as a result of defective desmosomal adhesion.

Jamora and Fuchs recently put forth the notion that the differential expression of desmosomal cadherins in the epidermis and HF imply a broader function for these proteins than simply as a “clamp between two cells” (Jamora and Fuchs, 2002). Likewise, the authors of the original description of the lanceolate mouse had postulated that “. . . a defective interaction between hair follicle adhesion molecules and keratins”, and moreover that “. . . a normal signaling molecule is missing or abnormal that periodically stimulates the follicle to continue in anagen” (Sundberg et al., 2000), thus predicting both a structural and a communication defect in the lanceolate HF.

We have uncovered a pivotal role for desmoglein 4 in keratinocyte cell adhesion, and moreover, in the execution of differentiation programs within the innermost keratinocyte populations of the HF, where the processes of mitosis, cell fate determination and intercellular adhesion must be seamlessly coordinated.

Since Desmoglein 4 has a role in hair shaft structure, and in its absence, only short and fragile hairs are formed, it is a rational target for pharmacologic inhibition. In contrast to Hairless protein inhibition, which causes damage to the hair follicle and permanent hair removal, inhibition of Desmoglein 4 does not damage the hair follicle itself, and only weakens the hair shaft. Therefore, Desmoglein 4 is more like Nude in terms of a drug target—i.e., inhibition of Desmoglein 4 expression will slow down hair growth, but not permanently remove it.

Catalytic Nucleic Acids

Catalytic nucleic acid technology is widely used to target mRNA in a sequence-specific fashion, and thus change the expression pattern of cells or tissues. While the goal of mRNA targeting is usually the cleavage of mutant mRNA with the prospect of gene therapy for inherited diseases, in certain instances targeting of wild-type genes can be used therapeutically.

This invention demonstrates the feasibility of using ribozyme and deoxy-ribozyme technology to alter gene expression in the skin via topical application and provide permanent hair removal.

Deoxy-ribozyme design and in vitro testing. To target the Desmoglein 4-encoding mRNA, a series of deoxy-ribozymes are designed based on the consensus cleavage sites 5′-RY-3′in the mRNA sequence. Those potential cleavage sites which are located on an open loop of the mRNA according to the RNA folding software RNADRaw 2.1 are targeted (Matzura and Wennborg 1996). The deoxy-ribozyme design utilizes the previously described structure (Santoro and Joyce 1997; Santoro and Joyce 1998) where two sequence-specific arms were attached to a catalytic core based on the Desmoglein 4-encoding mRNA sequence. The deoxy-ribozymes can be custom synthesized (e.g., by a laboratory such as Life Technologies). Commercially available mouse brain polyA-RNA (Ambion) serves as a template in the in vitro cleavage reaction to test the efficiency of the deoxy-ribozymes. For example, 800 ng RNA template can be incubated in the presence of 20 mM Mg2⁺ and RNAse Out RNAse inhibitor (Life Technologies) at pH 7.5 with 2 μg deoxy-ribozyme for one hour. After incubation, aliquots of the reaction are used as templates for RT-PCR, amplifying regions including the targeted cleavage sites. The RT-PCR products are visualized on an ethidium bromide-containing 2% agarose gel under UV light, and the intensity of the products is determined.

Deoxy-ribozyme treatment schdule. For each treatment, 2 μg deoxy-ribozyme, dissolved in a 85% EtOH and 15% ethylene glycol vehicle, can be applied to a one square centimeter area on the back.

Ribozymes can be delivered exogenously, such that the ribozymes are synthesized in vitro. They are usually administered using carrier molecules (Sioud 1996) or without carriers, using ribozymes specially modified to be nuclease-resistant (Flory et al. 1 996). The other method is endogenous delivery, in which the ribozymes are inserted into a vector (usually a retroviral vector) which is then used to transfect target cells. There are several possible cassette constructs to choose from (Vaish et al. 1998), including the widely used Ul snRNA expression cassette, which proved to be efficient in nuclear expression of hammerhead ribozymes in various experiments (Bertrand et al. 1997; Michienzi et al.1996; Montgomery and Dietz 1997).

Recent efforts have led to the successful development of small DNA oligonucleotides that have a structure similar to the hammerhead ribozyme (Santoro and Joyce 1997). These molecules are known as “deoxy-ribozymes,” “deoxyribozymes” and “DNAzymes,” and are virtually DNA equivalents of the hammerhead ribozymes. They consist of a 15-bp catalytic core and two sequence-specific arms with a typical length of 5-13 bp each (Santoro and Joyce 1998). Deoxy-ribozymes have more lenient consensus cleavage site requirements than hammerhead ribozymes, and are less likely to degrade when used for in vivo applications. The most widely used type of these novel catalytic molecules is known as the “10-23” deoxy-ribozyme, whose designation originates from the numbering used by its developers (Santoro and Joyce 1997). Because of their considerable advantages, deoxy-ribozymes have already been used in a wide spectrum of in vitro and in vivo applications (Cairns et al.2000; Santiago et al. 1999).

Antisense Nucleic Acids

Antisense oligodeoxynucleotides are synthesized as directed to the inhibition of Desmoglein 4 expression based on the Desmoglein 4-encoding mRNA sequence. Antisense oligonucleotides are chosen which are sufficiently complementary to the target, i.e., hybridize sufficiently well and with sufficient specificity, to give the desired disruption of the function of the molecule. “Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the antisense oligonucleotide. Similarly, catalytic nucleic acids are synthesized once cleavage target sites on the Desmoglein 4-encoding mRNA molecule have been identified, e.g., any purine:pyrimidine consensus sequences in the case of DNA enzymes.

Methods for selecting which particular antisense oligonucleotides sequences directed towards a particular protein-encoding mRNA are that will form the most stable DNA:RNA hybrids within the given target mRNA sequence are known in the art and are exemplified in U.S. Pat. No. 6,183,966 which is herein incorporated by reference.

In one embodiment at least one internucleoside linkage within the instant oligonucleotide comprises a phosphorothioate linkage. Antisense oligonucleotide molecules synthesized with a phosphorothioate backbone have proven particularly resistant to exonuclease damage compared to standard deoxyribonucleic acids, and so they are used in preference. A phosphorothioate antisense oligonucleotide for Desmoglein 4-encoding mRNA can be synthesized on an Applied Biosystems (Foster City, Calif.) model 380B DNA synthesizer by standard methods. For example, sulfurization can be performed using tetraethylthiuram disulfide/acetonitrile. Following cleavage from controlled pore glass support, oligodeoxynucleotides can be base deblocked in ammonium hydroxide at 60° C. for 8 h and purified by reversed-phase HPLC [0.1M triethylammonium bicarbonate/acetonitrile; PRP-1 support]. Oligomers can be detritylated in 3% acetic acid and precipitated with 2% lithiumperchlorate/acetone, dissolved in sterile water and reprecipitated as the sodium salt from 1 M NaCl/ethanol. Concentrations of the full length species can be determined by UV spectroscopy. Any other means for such synthesis known in the art may additionally or alternatively be employed. It is well known to use similar techniques to prepare oligonucleotides such as the phosphorothioates and alkylated derivatives.

Materials and Methods

Linkage Analysis:

Blood samples from family members were collected following informed consent, and genomic DNA was extracted using the PureGene DNA Isolation Kit (Gentra Systems).

Microsatellite markers were chosen from the Marshfield genetic map (http://research.marshfieldclinic.org/genetics/). A fully penetrant recessive model with no phenocopies and disease allele frequency of 0.001 was assumed. Marker alleles were re-coded using the RECODE program (ftp://watsonhgenedu/pub/recodetarZ). Two-point analyses were carried out using the MLINK program of the FASTLINK suite of programs (Lathrop et al., 1984) and multipoint and haplotype analyses using the SIMWALK program version 2.82 (Sobel and Lange, 1996). Recombination distances between markers were obtained from the sex-averaged Marshfield genetic map.

Genomic Structure of Desmoglein 4:

We analyzed the region on mouse chromosome 18 containing the desmosomal cadherin cluster (http://genome.ucsc.edu/; February 2002 Freeze). Analysis-of three open reading frames, Ensembl 00000037563, Geneid CHR18_{—}197, and Genscan CHR18_{—}2. 430, was used to predict the genomic structure of Dsg4. Sequencing of cDNA from mouse skin RNA and genomic DNA of PWK strain confirmed the sequence and identified an additional exon. The final cDNA sequence of Dsg4 was deposited under GenBank accession number AY227349.

Using the BLAT sequence analysis tool at http://genome.ucsc.edu/(December 2001 Freeze), we identified four human gene predictions homologous to the mouse Dsg4 cDNA within the human desmosomal gene cluster. Two of them, Ensembl ENST00000280910 and Fgenesh++ C18000296, were used to assemble a human DSG4 gene prediction. The final sequence was confirmed by sequencing of cDNA from human epithelial RNA and from genomic DNA, and is deposited under GenBank accession number AY227350. Amino acid identity and homology values were calculated using the NCBI blastp software (http://www.ncbi.nlm.nih.gov/BLAST/). For alignment of the four human desmoglein amino acid sequences we used the Clustal X software (Thompson et al., 1999).

Mutation Screening and RT-PCR:

All exons and splice sites were PCR amplified from genomic DNA from human LAH patients and controls, as well as lah/lah, lahJ/lahJ and control animals. PCR products were directly sequenced in an ABI Prism 310 sequencer. Dsg4 cDNA was RT-PCR amplified from control and mutant whole skin RNA using the following primers: DSG4 cDNA1F (5′ TCTCCTAGTACAGCCTGCTT 3′; SEQ ID NO: 7) and Dsg4 cDNA8R (5′ AGTGGTCTCTCCAAGTCTTC 3′; SEQ ID NO: 8), corresponding to the first exons of Dsg4. The potential phosphorylation of Y196 was predicted using software available at www.cbs.dtu.dk/services/NetPhos/.

Northern analysis and In Situ Hybridization:

Two μg normal human skin poly(A) RNA (Stratagene) was transferred to Nylon membranes (Amersham) (Sambrook et al., 1989). Human and mouse multiple tissue blots containing 2 mg poly(A) RNA per lane were purchased from Ambion and OriGene Technologies INC, respectively. The human blots were hybridized with [32P] labeled cDNA probe corresponding to human DSG4 exons 3-8 amplified using primers DSG4 cDNA3F (5′ AGTTTGCCGCAGCCTGTCGA 3′; SEQ ID NO: 9) and DSG4 cDNA8R (5′ CCAGTTATCAGTGCCTTCTTC 3′; SEQ ID NO: 10). The mouse blots were hybridized with a [32P] labeled cDNA probe corresponding to Dsg4 exons 4-8 amplified using primers Dsg4 cDNA 4F (5′ TTGATCGGCCACCTTACGG 3′; SEQ ID NO: 11) and Dsg4 cDNA 8R (5′ CCAACCAGTTATCAGTGCCT 3′; SEQ ID NO: 12). The hybridizations were carried out suing Rapid Hyb buffer (Amersham).

In situ hybridization was performed on 4% PFA fixed 4 mm frozen sections from Balb/c adult mice with DIG labeled Dsg4 riboprobes (Roche Molecular Biochemicals), as described elsewhere (Mendelsohn et al., 1999). After developing the signal with NBT/BCIP substrate, slides were dehydrated and mounted in Shandon mounting medium (Thermoshandon).

DSG4 Antibody Synthesis Immunofluorescence Microscopy Western blot:

Polyclonal antibodies for human DSG4 were raised in chicken against the following peptide: ‘N’-NATSAILTALQVLSPGFYEIPI-‘C’ (SEQ ID NO: 13) (Washington Biotechnology). Other antibodies were as follows: b-catenin (1:100) (Sigma, St. Louis, Mo.); K1 (1:500), K5 (1:1000), K6 (1:500), loricrin (1:500), involucrin (1:1000), diphosphorylated Erk1/2 (1:50) (Babco); hoxc13 (1:800), Ha4 (1:200) and hb2 (1:2000) (generous gift from Dr. Jurgen Schweitzer); a6 integrin (1:50), b1 integrin (1:50) (Chemicon), Dsg1 (1:100), Dsg3 (1:30), P-cadherin (1:50), and EGFR (1:50) and ERK 1 /2 (1:100) (Santa Cruz); E-cad (1:50) (ED Transduction laboratories); Pg (1:50) and Pkp1 (1:100) (Zymed); PCNA (1:50) (Oncogene Research Products); Dsp (1:20) and pan-desmocollin (1:50) (generous gift from Dr. My Mahoney); nude (Foxn1) (1:30) (generous gift from Dr. Janice Brissette).

Human scalp and mouse dorsal skin sections of day 8 lahJ/lahJ or WT littermates were fixed in either acetone at −20° C. for 10 mins or 4% PFA in PBS at room temperature for 10 mins. Immunofluorescent staining was performed as described previously for both cells and frozen sections (Harlow and Lane, 1998). For mouse monoclonal antibodies, the M.O.M. kit was used for immunofluorescence and Mouse Elite Kit was used for immunihistochemistry (Vector Laboratories).

Recombinant protein of an N-terminal region of DSG4 was expressed in SG13009 bacteria using pQE30 expression vector (Nguyen et al., 2000). Recombinant protein was affinity purified with Qiagen Ni-NTA Spin column and used for Western blot analysis of sera from PV patients or healthy individuals. Binding of primary antibodies was recognized by HRP-conjugated goat anti-human IgG secondary antibody.

Transmission Electron Microscopy:

Skin from dorsal back of day 14 lahJ/lahJ and WT littermates was fixed in half-strength Karnovsky's fixative (2% PFA/2.5% glutaraldehyde phosphate buffer) followed by fixation in 1.3% osmium tetroxide. Samples were processed using standard TEM techniques and mounted in Epon resin. Ultrathin sections were collected on grids and stained with uranyl acetate and lead citrate. Sections were visualized using a Jeol 100CX transmission electron microscope.

Primary Mouse Keratinocyte Culture:

Mouse keratinocytes were isolated and cultured as described (Morris et al., 1994), with minor modifications. 2×10⁶ cells per dish were plated onto 35 mm dishes (Becton Dickinson) with vitrogen-fibronectin coating and cultures were kept in a 32° C. humidified incubator. For high Ca⁺⁺ conditions, a final concentration of 1.2 mM was used on day 4-5 cultures. For immunostains, the cells were fixed in ice cold methanol at −20° C. for 10 minutes. The attachment assay was performed 24 hrs after seeding in low Ca⁺⁺ medium (Freshney, 1987) on triplicate plates. Cells were trypsinized with 0. 25% trypsin for 4 mins at 32° C., collected by centrifugation, and counted using a hemocytometer.

### REFERENCES

Bartel D P (1999) Creation and evolution of new ribozymes. Biol Bull 196: 322-3.
Bertrand E, Castanotto D, Zhou C, Carbonnelle C, Lee N S, Good P, Chatterjee S, Grange T, Pictet R, Kohn D, Engelke D, Rossi J J (1997) The expression cassette determines the functional activity of ribozymes in mammalian cells by controlling their intracellular localization. RNA 3: 75-88.
Cech T R (1987) The chemistry of self-splicing RNA and RNA enzymes. Science 236: 1532-9.
Fedor M J (2000) Structure and Function of the Hairpin Ribozyme. J Mol Biol 297 (2): 269-91.
Flory C M, Pavco P A, Jarvis T C, Lesch M E, Wincott F E, Beigelman L, Hunt S W, 3rd, Schrier DJ (1996) Nuclease-resistant ribozymes decrease stromelysin mRNA levels in rabbit synovium following exogenous delivery to the knee joint. Proc Natl Acad Sci USA 93: 754-8.
Matzura O, Wennborg A (1996) RNAdraw: an integrated program for RNA secondary structure calculation and analysis under 32-bit Microsoft Windows. Comput Appl Biosci 12: 247-9.
Montgomery R A, Dietz H C (1997) Inhibition of fibrillin 1 expression using U1 snRNA as a vehicle for the presentation of antisense targeting sequence. Hum Mol Genet 6: 519-25.
Pley et al, (1994) Three-dimensional structure of a hammerhead ribozyme. Nature 372: 68-74.
Phylactou L A, Kilpatrick M W, Wood M J (1998) Ribozymes as therapeutic tools for genetic disease. Hum Mol Genet 7: 1649-53.
Sambrook et al., “Molecular Cloning: A Laboratory Manual”, Second Edition (1989), Cold Spring Harbor Laboratory Press, Cold Spring Harbor, N.Y.

Santoro S W, Joyce G F (1997) A general purpose RNA-cleaving DNA enzyme. Proc Natl Acad Sci USA 94: 4262-6.
Santoro S W, Joyce G F (1998) Mechanism and utility of an RNA-cleaving DNA enzyme. Biochemistry 37: 13330-42.
Vaish N K, Kore A R, Eckstein F (1998) Recent developments in the hammerhead ribozyme field. Nucleic Acids Res 26: 5237-42.
Ahmad, W., ul Haque, M. F., Brancolini, V., Tsou, H. C., ul Haque, S., Lam, H., Aita, V. M., Owen, J., deBlaquiere, M., Frank, J., et al. (1998). Alopecia universalis associated with a mutation in the human hairless gene. Science 279, 720-724.
Armstrong, D. K., McKenna, K. E., Purkis, P. E., Green, K. J., Eady, R. A., Leigh, I. M., and Hughes, A. E. (1999). Haploinsufficiency of desmoplakin causes a striate subtype of palmoplantar keratoderma. Hum Mol Genet 8, 143-148.
Auber, L. (1952). The anatomy of follicles producing wool-fibers, with special reference to keratinization. Trans Roy Soc Edin 62, 191-254.
Bullough, W. S., and Laurence, E. B. (1958). The mitotic activity of the follicle. In The biology of hair growth, W. a. E. Montagna, R. A., ed. (New York, Academic Press), pp. 171-187.
Buxton, R. S., Cowin, P., Franke, W. W., Garrod, D. R., Green, K. J., King, I. A., Koch, P. J., Magee, A. I., Rees, D. A., Stanley, J. R., and et al. (1993). Nomenclature of the desmosomal cadherins. J Cell Biol 121, 481-483.
Carroll, J. M., Romero, M. R., and Watt, F. M. (1995). Suprabasal integrin expression in the epidermis of transgenic mice results in developmental defects and a phenotype resembling psoriasis. Cell 83, 957-968.
Chidgey, M., Brakebusch, C., Gustafsson, E., Cruchley, A., Hail, C., Kirk, S., Merritt, A., North, A., Tselepis, C., Hewitt, J., et al.(2001). Mice lacking desmocollin 1 show epidermal fragility accompanied by barrier defects and abnormal differentiation. J Cell Biol 155,821-832.
Eshkind, L., Tian, Q., Schmidt, A., Franke, W. W., Windoffer, R., and Leube, R. E. (2002) Loss of desmoglein 2 suggests essential functions for early embryonic development and proliferation of embryonal stem cells. Eur J Cell Biol 81,592-598.
Frank, J., Pignata, C., Panteleyev, A. A., Prowse, D. M., Baden, H., Weiner, L., Gaetaniello, L., Ahmad, W., Pozzi, N., Cserhalmi-Friedman, P. B., et al. (1999). Exposing the human nude phenotype. Nature 398,473-474.
Freedberg, I. M., Tomic-Canic, M., Komine, M., and Blumenberg, M. (2001). Keratins and the keratinocyte activation cycle. J Invest Dermatol 116,633-640.
Freshney, R. I. (1987). Animal cells: a manual of basic technique. (New York, Wiley-Liss, Inc.).
Frischmeyer, P. A., and Dietz, H. C. (1999). Nonsense-mediated mRNA decay in health and disease. Hum Mol Genet 8, 1893-1900.
Fuchs, E., Merrill, B. J., Jamora, C., and DasGupta, R. (2001). At the roots of a never-ending cycle. Dev Cell 1, 13-25.
Garrod, D. R., Merritt, A. J., and Nie, Z. (2002). Desmosomal cadherins. Curr Opin Cell Biol 14,537-545.
Green, K. J., and Gaudry, C. A. (2000). Are desmosomes more than tethers for intermediate filaments? Nat Rev Mol Cell Biol 1,208-216.
Grinnell, F. (1990). The activated keratinocyte: up regulation of cell adhesion and migration during wound healing. J Trauma 30, S144-149.
Hardy, M. H. (1992). The secret life of the hair follicle. Trends Genet 8,55-61.
Harlow, E, and Lane, D. (1998). Using antibodies: a laboratory manual (New York, Cold Spring Harbor Laboratory Press).
Hudson, L. G., and McCawley, L. J. (1998). Contributions of the epidermal growth factor receptor to keratinocyte motility. Microsc Res Tech 43,444-455.
Hunt, D. M., Rickman, L., Whittock, N. V., Eady, R. A., Simrak, D., Dopping-Hepenstal, P. J., Stevens, H. P., Armstrong, D. K., Hennies, H. C., Kuster, W., et al.(2001). Spectrum of dominant mutations in the desmosomal cadherin desmoglein 1, causing the skin disease striate palmoplantar keratoderma. Eur J Hum Genet 9,197-203.
Jamora, C., and Fuchs, E. (2002). Intercellular adhesion, signalling and the cytoskeleton. Nat Cell Biol 4, E101-108.
Jiang, C. K., Magnaldo, T., Ohtsuki, M., Freedberg, I. M., Bernerd, F., and Blumenberg, M. (1993). Epidermal growth factor and transforming growth factor alpha specifically induce the activation-and hyperproliferation-associated keratins 6 and 16. Proc Natl Acad Sci USA 90, 6786-6790.

Kljuic, A., and Christiano, A. M. (2003). A novel mouse desmosomal cadherin family member, desmoglein Ig. Exp Derm 12,20-29.
Kljuic, A., Gilead, L., Martinez-Mir, A., Frank, J., Christiano, A. M., and Zlotogorski, A. (In Press). A Nonsense Mutation in the Desmoglein 1 Gene Underlies Striate Keratoderma. Exp Dermatol.
Koch, P. J., Mahoney, M. G., Ishikawa, H., Pulkkinen, L., Uitto, J., Shultz, L., Murphy, G. F., Whitaker-Menezes, D., and Stanley, J. R. (1997). Targeted disruption of the pemphigus vulgaris antigen (desmoglein 3) gene in mice causes loss of keratinocyte cell adhesion with a phenotype similar to pemphigus vulgaris. J Cell Biol 137, 1091-1102.
Kurose, K., Mori, O., Hachisuka, H., Shimizu, H., Owaribe, K., and Hashimoto, T. (2000). Cultured keratinocytes from plectin/HD1-deficient epidermolysis bullosa simplex showed altered ability of adhesion to the matrix. J Dermatol Sci 24, 184-189.
Kurzen, H., Moll, I., Moll, R., Schafer, S., Simics, E., Amagai, M., Wheelock, M. J., and Franke, W. W. (1998). Compositionally different desmosomes in the various compartments of the human hair follicle. Differentiation 63, 295-304.
Lathrop, G M., Lalouel, J. M., Julier, C., and Ott, J. (1984). Strategies for multilocus linkage analysis in humans. Proc Natl Acad Sci USA 81, 3443-3446.
McGrath, J. A., McMillan, J. R., Shemanko, C. S., Runswick, S. K., Leigh, I M., Lane, E. B., Garrod, D. R., and Eady, R. A. (1997). Mutations in the plakophilin 1 gene result in ectodermal dysplasia/skin fragility syndrome. Nat Genet 17, 240-244.
McKoy, G., Protonotarios, N., Crosby, A., Tsatsopoulou, A., Anastasakis, A, Coonar, A., Norman, M., Baboonian, C., Jeffery, S., and McKenna, W. J. (2000). Identification of a deletion in plakoglobin in arrhythmogenic right ventricular cardiomyopathy with palmoplantar keratoderma and woolly hair (Naxos disease). Lancet 355, 2119-2124.
McMillan, J. R., and Shimizu, H. (2001). Desmosomes: structure and function in normal and diseased epidermis. J Dermatol 28, 291-298.
Mendelsohn, C., Batourina, E., Fung, S., Gilbert, T., and Dodd, J. (1999). Stromal cells mediate retinoid-dependent functions essential for renal development. Development 126, 1139-1148.
Montagutelli, X., Hogan, M. E., Aubin, G., Lalouette, A., Guenet, J. L., King, L. E., Jr., and Sundberg, J. P. (1996). Lanceolate hair (lah): a recessive mouse mutation with alopecia and abnormal hair. J Invest Dermatol 107, 20-25.
Montagutelli, X., Lalouette, A., Boulouis, H. J., Guenet, J. L., and Sundberg, J. P. (1997). Vesicle formation and follicular root sheath separation in mice homozygous for deleterious alleles at the balding (bal) locus. J Invest Dermatol 109, 324-328.
Nguyen, V. T., Ndoye, A., Shultz, L. D., Pittelkow, M. R., and Grando, S. A. (2000). Antibodies against keratinocyte antigens other than-desmogleins 1 and 3 can induce pemphigus vulgaris-like lesions. J Clin Invest 106, 1467-1479.
Norgett, E. E., Hatsell, S. J., Carvajal-Huerta, L., Cabezas, J. C., Common, J., Purkis, P. E., Whittock, N., Leigh, I. M., Stevens, H. P., and Kelsell, D. P. (2000). Recessive mutation in desmoplakin disrupts desmoplakin-intermediate filament interactions and causes dilated cardiomyopathy, woolly hair and keratoderma. Hum Mol Genet 9, 2761-2766.
Orwin, D F. (1979). The cytology and cytochemistry of the wool follicle. Int Rev Cytol 60, 331-374.
Paine-Saunders, S., Viviano, B. L., Economides, A. N., and Saunders, S. (2002). Heparan sulfate proteoglycans retain Noggin at the cell surface: a potential mechanism for shaping bone morphogenetic protein gradients. J Biol Chem 277, 2089-2096.
Pulkkinen, L., Choi, Y. W., Kljuic, A., Uitto, J., and Mahoney, M. G. (2003). Novel member of the mouse desmoglein family: Dsg1-6. Exp Derm 12, 11-19.
Pulkkinen, L., Choi, Y. W., Simpson, A., Montagutelli, X., Sundberg, J., Uitto, J., and Mahoney, M. G. (2002). Loss of cell adhesion in Dsg3bal-Pas mice with homozygous deletion mutation (2079de114) in the desmoglein 3 gene. J Invest Dermatol 119, 1237-1243.
Rikimaru, K., Moles, J. P., and Watt, F M. (1997). Correlation between hyperproliferation and suprabasal integrin expression in human epidermis reconstituted in culture. Exp Dermatol 6, 214-221.
Roth, S. I., and Helwig, E. B. (1964). The Cytology of the Dermal Papilla, the Bulb, and the Root Sheaths of the Mouse Hair. J Ultrastructure Res 11, 33-51.
Sobel, E., and Lange, K. (1996). Descent graphs in pedigree analysis: applications to haplotyping, location scores, and marker-sharing statistics. Am J Hum Genet 58, 1323-1337.
Sundberg, J. P., Boggess, D., Bascom, C., Limberg, B. J., D., S. L., A., S. B., Jr., K. L. E., and Montagutelli, X. (2000). Lanceolate hair-J (lahJ) : a mouse model for human hair disorders. Exp Dermatol 9, 206-218.
Thompson, J. D., Plewniak, F., and Poch, 0. (1999). A comprehensive comparison of multiple sequence alignment programs. Nucleic Acids Res 27, 2682-2690.
Tong, X., and Coulombe, P. A. (2003). Mouse models of alopecia: identifying structural genes that are baldly needed. Trends Mol Med 9, 79-84.
Van Scott, E. J., Ekel, T. M., and Auerbach, R. (1963). Determinants of rate and kinetics of cell division in scalp hair. J Invest Dermatol 4, 269-273.

### Example 2

Rodents with spontaneous skin and hair mutations are becoming increasingly valuable as models of human disease and in the understanding of the complex biology of skin and hair follicles. The Jackson Laboratory lists a large number of mouse mutations alone with defects whose mutations have not been identified [1, 2]. Likewise, there exists a small number of rat models of hypotrichosis which have also not been characterized at the molecular level [3, 4]. Such models are widely used in the study of the treatment of dermatological diseases and the efficacy of topical medications [5, 6], but are rarely studied as primary models for the understanding of the mechanisms of hair shaft of cycling defects.

In recent years, comparative genomics between the rodent and human genomes have uncovered several examples of mutations in orthologous genes that underlie similar phenotypes. These include the hairless gene underlying Atrichia with Papular Lesions in humans (OMIM 209500) and the hairless and rhino mouse [7-9], as well as mutations in the nude gene in Alopecia with T Cell Immunodeficiency (OMIM 601705), allelic with the nude mouse [10, 11]. In these instances, the relationships between the mouse and human phenotypes have been made due to phenotypic similarities, genetic linkage studies, and identification of the gene in both species. While several mouse mutations have been described that result from mutations in transcription factors and secreted proteins, such as the hairless, nude and angora phenotypes, even fewer animal models with spontaneous mutations in structural proteins have been described [12]. A notable example is the balding mouse mutation (bal) resulting from mutations in the Desmoglein 3 gene (Dsg3), which resides in the desmosomal cadherin gene cluster and is expressed in epidermis and hair follicle, but for which no human counterpart yet exists [13, 14].

We have extended our work on the comparative genomic approach to human disease in studying the lanceolate hair mouse (lah) model, which had previously been mapped to mouse chromosome 18 [15,16]. We have identified a human disorder, localized autosomal recessive hypotrichosis (LAH) (OMIM 607903), which bears striking resemblance to the lah/lah mouse mutation, and which we subsequently mapped to the syntenic region of human chromosome 18. A positional cloning strategy combined with in silico approaches revealed the unexpected presence of a new member of the desmosomal cadherin family, which was designated Desmoglein 4 in the mouse (Dsg4) and human (DSG4). We recently identified mutations in the Desmoglein 4 gene in two human families with LAH, as well as both of the lanceolate hair alleles, lah/lah and lahJ/lahJ. The phenotypic similarities are typified by the presence of sparse, fragile broken hair shafts which form a lance head at the tip, leading to the designation of the phenotype as lanceolate hair.

In this study, we discovered a spontaneous autosomal recessive rat mutation with a phenotype reminiscent of the lanceolate hair mutation, which we have therefore named lah/lah. This line of rats was derived from a single mutant animal originally observed in a BDIX breeding colony in Leeds, UK. Given the phenotypic similarities between this rat model and the lanceolate hair mouse, we cloned the rat homologue of Dsg4, and subsequently identified a homozygous missense mutation in the lah/lah rat. Interestingly, this mutation resides directly within the calcium coordinating pocket within the extracellular domain of Dsg4, and is predicted to interfere with extracellular assembly of cadherin partners [18]. At the cellular level, this mutation appears to cause an increase in cell proliferation in the epidermis, as well as the upregulation of several classic markers of hyperproliferation. The discovery of a mutation in the Desmoglein 4 gene in the lah/lah rat provides a new animal model for the study of inherited hypotrichosis in humans, and allows for analysis of Desmoglein 4 in the in vivo setting.

### EXPERIMENTAL RESULTS

Hypotrichosis in lah/lah Rats

The lah/lah rats are born naked with pink, wrinkled skin and are distinguishable from normal brown BDIX rats at birth by their relatively small size. The vibrissae and first hair coat appear around day five, with the skin developing a dark, gray, stubble-like hue. Hair growth then progresses from the head to tail region with the rat developing a full coat of pelage hair around two weeks. At this stage they are still distinguishable from brown rats by size and coloration. Hair loss begins shortly afterwards and culminates around four weeks when the rats are completely bald. Hair re-growth starts again a few days later, following an approximately twenty nine day cycle of external growth and loss generally from head to tail with ventral to dorsal change as well. In some animals the region around the eyes (sometimes extending in a line to the neck) is spared in early cycles. Hair loss can be heterogeneous, even between littermates although no patterns of difference were seen between sexes. Hair loss and cycling was almost synchronous in young rats but less so with increasing age. With each subsequent growth cycle hair regrowth is less significant and it becomes increasingly patchy and “stubbly.” Almost complete hair pelage hair loss occurs by eighteen months, although in these rats the skin still undergoes cyclical changes alternating between a dark gray and pink/yellow color, indicating that follicle remnants are still cycling. Vibrissa follicles continued to produce whisker fibers throughout, but these were sometimes abnormally shaped and grew in unusual directions.

lah/lah Hair Fiber Abnormalities

Detailed examination of the skin and hair of affected animals revealed three main fiber colors, dark black fibers, brown/orange fibers and white transparent fibers. In areas where the balding process was advanced such as the stomach and thighs, and generally in older rats, only black and white hairs were seen, presumably accounting for the unusual gray hue of these animals. Fibers revealed striking thickening or nodules often at their tips, suggesting initially that this was an effect occurring in early anagen. However, high resolution showed that in many cases the nodules were some distance down the hair, so it was likely that in others the tips had broken off. Intriguingly, the distal growth was unpigmented, therefore this dramatic fiber thickening coincided with the switching on of fiber pigmentation. Plucked fibers confirmed these features and preliminary counts and characterization suggested that all fiber types displayed the nodular phenomenon (data not shown). All hairy regions of the body including ears showed fibers with nodules, and only tail hairs remained largely in place.

The formation of Lanceolate Hairs in the lah/lah Rat

In contrast to unaffected animals whose skin had a normal histological appearance, affected animals displayed many unusual features. Particularly evident were unusual directional growth of fibers, acute angling or twisting of, shafts, root sheath hyperplasia and multiple hairs growing in a single expanded shaft. In anagen follicles from the second cycle onwards, the characteristic nodules were seen in many pelage hair shafts and with increasing age, follicle structure became increasingly irregular. Several abnormalities were observed in follicle bases, including the loss of the fiber in follicles that were still in anagen, and some very unusual bulb structures. Often these had the anatomical appearance of follicles that had been recently plucked, an indication perhaps of inherent weakness or fragility in the follicle epithelium, at or around the line of Auber. In older animals a few residual follicles were left in a thickened dermis, and interestingly isolated dermal papilla cell clumps were sometimes visible deep in the dermis intact indicating that when the epithelial components of the follicle had been destroyed or separated off this had remained. No immunological infiltrates were seen in association with the follicles.

Similar to the lanceolate hair mouse models [15,16], the first signs of the lah phenotype emerged in anagen when the formation of the swelling of the hair shaft in the precortical region was observed, which is the hallmark of the lah/lah phenotype. The swelling is believed to be the result of disrupted cell adhesion between the rapidly dividing matrix cells at the base of the follicle, which leads to a failure of differentiation into the different hair follicle layers [15-17]. Improper hair shaft differentiation is thought to lead to the formation of the keratinous mass that eventually forms the lance head, as well as the long thin transparent tail that emerges from the hair canal preceding the tip of the lance head. The differential pigmentation of the tail and the abnormal hair shaft may be the result of impaired uptake of pigment granules in the matrix of the hair follicle, perhaps secondary to the cell adhesion defect.

Unlike the lahJ/lahJ Dsg4-null mutants, which die at around the time of weaning, the longer lifespan of the lah/lah rat allowed us to follow the hair and skin phenotype for longer periods of time. In adult animals, we noticed the presence of several defects that we ascribe to being secondary changes after the initial destruction of the hair. These include large included cysts with coiled embedded hairs, ruptured follicles, and enlarged hair canals filled with sebum. Our interpretation of these findings is that they are the end-products of the massive degenerative process that takes place within lah/lah hair follicles.

Desmoglein 4 a Novel Desmosomal Cadherin Family Member in the Rat Genome

Using the BLAST software at the NIH genome database site, we identified two rat BAC clones that contained sequences corresponding to Dsg4 exons. Based on this sequence, we designed PCR primers and amplified all 16 exons and the corresponding exon/intron boundaries from lah/lah rat genomic DNA.

The rat cDNA for Dsgs4 consists of 3123 bp encoding a protein of 1040 amino acids (GenBank accession number AY314982). At the amino acid level, the rat Dsg4 shares 77% and 91% amino acid identity to human and mouse Desmoglein 4, respectively, and 84% and 92% homology. Rat Dsg4 exhibits all the hallmarks of a desmosomal cadherin [19,20]. It has four N-terminal extracellular cadherin repeats (EI-EIV), followed by an extracellular anchoring domain (EA), a transmembrane domain (TM), an intracellular anchoring domain (IA), an intracellular cadherin specific sequence (ICS), a linker domain (LD), three intracellular repeated unit domains (RUD), and a terminal domain (TD) at the carboxyl end. Notable sequence motifs in human, mouse and rat Desmoglein 4 include the presence of an RXKR (SEQ ID NO:14) motif at amino acids 46-49, representing the proteolytic processing site of convertases found in both classical and desmosomal cadherins [19,20]. A RAL (SEQ ID NO:15) tripeptide sequence located at amino acids 128-130, represents the potential site for cadherin interaction. We detected five putative calcium binding sites (DXNDN; SEQ ID NO:16 or A/VXDXD; (SEQ ID NO:17) and five sites for N-linked glycosylation (NXS/T; SEQ ID NO:18). Desmoglein 4 also contains three conserved repeats, which define the RUD, with the core repeat sequences being DIIVTE (SEQ ID NO:19), NVVVTE (SEQ ID NO:20), and NVIYAE (SEQ ID NO:21) (NVYYAE, SEQ ID NO:22 in mouse) [19,20]. These elements are found in all desmogleins, however, their biological significance is unknown.

Interestingly, the desmosomal cadherin gene cluster in rat is arranged similarly to that in the human genome with seven desmosomal cadherins arranged in the following order: Dsc3-Dsc2-Dsc1-Dsg1-Dsg4-Dsg3-Dsg2 and spans 550 kb. Recently, we discovered two homologs of the Dsg1 gene in the mouse genome, and designated these two new genes, Dsglβ [21] and Dsglγ [22]. These two genes flank the originally described Dsg1 gene (now referred to as Dsglα) and reside between the Dscl and Dsg4 genes in the mouse genome. It is noteworthy that Dsglβ, and Dsglγ are not found in either the human or rat genomes. The finding of only a single Dsgl gene in the rat genome suggests that Dsglβ and Dsglγ genes were lost in mammalian evolution between mouse and rat. Recent reports estimate the split between the two organisms could have occurred as recently as 16-23 million years ago [23].

A Missense Mutation in Dsg4 Underlies the lah/lah Rat Phenotype

Sequence analysis of Dsg4 gene in lah/lah animals identified a homozygous A-to-T transversion at nucleotide 676. This mutation converted a glutamic acid residue (GAG) in exon 6 to a valine residue (GTG), designated E228V. Extensive BLAST searches and sequencing of 10 unrelated, unaffected rat control DNAs indicated that E228V is not a common polymorphism.

The glutamic acid at residue 228 is conserved in all other rat desmoglein genes as well as the human, mouse canine and bovine desmogleins. Furthermore, this residue is also conserved in desmocollins, classical cadherins, and other distantly related adhesion molecules such as D. melanogaster dachsous. This mutation resides 32 amino acids downstream within the same exon as our previously reported lah/lah mouse missense mutation, Y196S. Both mutations are localized within the second extracellular domain (EC2) of Dsg4, in a region that is responsible for adhesion between adjacent cells. Shown in is the alignment of this region of the desmogleins as well as highlighting the close proximity of the two mutations. Further support for the importance of this domain in desmoglein function comes from our recently reported human DSG4 mutation, which is comprised of a deletion of exons 5-8 of DSG4. This mutation is in-frame, and therefore results in an internally-deleted DSG4 polypeptide which is missing amino acids 125-335, including both Y196 and E228.

Disruption of a Calcium Binding Site in lah/lah Mutant Rats

The glutamic acid residue at position 228, mutated in the lah/lah rats, is part of an LDRE sequence (SEQ ID NO: 40)_known to play a central role in calcium coordination in all cadherins [24,25]. The extracellular segments of desmosomal cadherins, like the well-studied classic cadherins, are comprised of five tandemly-related extracellular cadherin (EC) domains, EC1-EC5 (EC5 is also referred to as EA-extracellular anchor domain). EC1 is at the N-terminus, and is the most membrane-distal module, while EC5 is near the membrane attachment point. Binding sites for three calcium ions are situated at each interface between successive cadherin domains; thus the whole ectodomain accommodates the binding of twelve calcium ions [24,25]. Calcium is necessary for cadherins to function in adhesion [26].

The molecular basis for this requirement appears to arise from the ability of calcium to stabilize the interdomain connections, thus to transform the cadherin extracellular domain from a collapsed globule in the absence of calcium, to a stiff rod in its presence [27]. Each interdomain linkage, in the absence of calcium, has a substantial negative charge arising from the concentration of glutamic and aspartic acid residues that function in calcium coordination. These pockets of spatially localized negative charge are likely unable to form a compact structure due to charge-charge repulsion [24,25, 27]. The binding of calcium ions—in addition to the specific bonds formed in ligation—are thought to neutralize the negative charge, thus to enable adoption of tightly folded junctions between successive domains, and stiffening of the cadherin ectodomain into its functional rod-like form.

The crystal structure of the ectodomain from C-cadherin [24] shows that the corresponding residue in that protein, E182, is of central importance in the EC2-EC3 interdomain calcium binding site. As in all known cadherin calcium binding sites [24,25, 28], the side chain of this glutamic acid residue ligates both Ca1 and Ca2. A mutation of this residue to the hydrophobic amino acid valine, as in the lah/lah rat, would almost certainly impair calcium binding, thus preventing the adoption of the native EC2-EC3 domain interface, and preventing the mutant protein from attaining its functional extended form.

Phenotypic Consequences of the Dsg4 Mutation in the lah/lah Rat

We first investigated the effects of Dsg4 mutation on interfollicular epidermis and, similar to lah/lah mouse mutants, found evidence of markers of an activated phenotype. We found increased cell proliferation using the marker Ki67, indicative of not only hyperproliferation but also the existence of dividing cells in the suprabasal layers of the epidermis where they are usually not found (not shown). This phenotype suggests a premature or disregulated exit of dividing cells from the basal compartment, and led us to test for the presence of two other markers of the hyperproliferative phenotype [29]. Accordingly, we found upregulation of epidermal growth factor receptor (EGFR) as well as keratin 6 in the suprabasal epidermis, providing further support for the activated state. In many mouse models, the appearance of K6 (an EGF target gene, [30]) and EGFR coincides with an inflammatory infiltrate, yet in the lah/lah rat as well as mice, we see no evidence for the presence of inflammation concomitant with the activation of proliferation [17, 31]. EGFR is also markedly expressed in the lah/lah hair follicle, whereas it is not expressed in wild-type follicles. Thus, the most consistent feature in both the hair follicle and the epidermis is the upregulation of EGFR and K6 in both compartments. This finding is interesting in light of the negative effect of EGF on hair shaft production in hair follicle organ culture [32]. As expected on the basis of the missense mutation, the expression of Dsg4 is unchanged between WT and lah/lah mutant animals.

The phenotype of the lah/lah rat is most reminiscent of the original lah/lah mouse mutation which harbors the missense mutation Y196S. In contrast to the null mutant, lahJ/lahJ, both the rat and mouse lah/lah mutations have a normal lifespan and develop very similar phenotypic changes. It is our hypothesis that the absence of Dsg4 in critical extra cutaneous tissues is responsible for the demise of the null animals, while the presence of a mutant Dsg4 protein, albeit imperfect, is sufficient for intermediate function and results in a non-lethal phenotype. Likewise, the presence of an internally-deleted yet in-frame mutation in our human LAH families also suggests that a mutant DSG4 protein is sufficient for the rescue of function in essential tissues, however, the hair phenotype is consistent throughout all mutants analyzed to date. The rare occurrence of mouse and now rat models for human LAH provides the opportunity to study the consequences of Desmoglein 4 mutations on several different backgrounds in the in vivo context.

Whether the upregulation of these markers is a direct consequence of mutant Dsg4, or a secondary effect resulting from epidermal disadhesion remains to be explored, however, the lah/lah rat provides a new model system for examining the role of Dsg4 in many cellular processes including cell adhesion, signaling, and perhaps the transmission of developmental and morphogenic signals.

### MATERIALS AND METHODS

Phenotypic observations. These were carried out at weekly intervals and sometimes more frequently depending on the stage of the hair cycle. Affected animals from particular litters were examined and photographed and compared with unaffected animals from the same litter.

Histology and investigation of hair fiber characteristics. Affected animals of both sexes were sacrificed at different intervals. For histology, skin biopsies were removed from different points from the head to tail of animals and from the mystacial pad region containing the vibrissa follicles. Specimens were then processed for routine wax histology, and sections staine with Weigert's Hematoxylin, Curtis' ponceau S and Alcian Blue. Images were obtained from a Zeiss Axiovert 135 microscope equipped with a Spot RT slider digital camera (Diagnostic Instruments). Fiber characteristics were examined in different regions of. the body using a Zeiss SV 11 microscope fitted with the same digital camera. In given areas, fibers were also plucked and examined in order to gauge whether specific types were differentially affected.

Cloning of rat Desmoglein 4. The mouse Dsg4 cDNA sequence was used to BLAST rat genome sequences at and two BAC clones were identified with corresponding rat Dsg4 sequences. Sequences corresponding to Dsg4 exons 2, 3 and 15 were obtained from clone CH230-313J8 (AC112848.2) and sequences corresponding to all the remaining exons (1, 4-14, and 16) were obtained from clone CH230-279113 (AC111835.20). Based on the BAC clone sequences, we designed PCR primers to amplify across the rat Dsg4 exons. The rat dsg4 sequence has been deposited under GenBank accession # AY314982.

Mutation screening. All 16 exons and corresponding exon/intron boundaries of Dsg4 were amplified by PCR from control and lah/lah genomic DNA and sequenced. PCR amplifications were performed using Platinum Taq PCR Supermix (InVitrogen), 20 pmol of forward and reverse primers and approximately 500 ng of rat genomic DNA per reaction. PCR products were purified using Rapid PCR Purification System (Marligen Bioscience Inc.) and sequenced, using an ABI Prism 310 automated sequencing system (PE-Applied Biosystems), in both directions utilizing the same primers used for the initial PCR.

Immunofluorescence microscopy. Immunofluorescence staining of sections of lah/lah rat skin was performed as previously described. Briefly, 6 um sections were cut on the Leica cryostat, dried for 15 minutes and fixed in 4% PFA/0. 4% Triton X-100. Blocked for 30 minutes in 0. 2% Fish Skin Gelatin (Sigma)/0. 4W Triton X-100 in PBS. Primary and secondary antibodies were incubated in the same solution. Where required, propidium iodide or Hoechst dye (Sigma) were used as a nuclear counterstain. The following primary antibodies and dilutions were used: rabbit anti-cytokeratins 14/10 and 6 (Babco) 1/100, rabbit anti EGFR (Santa Cruz Biotechnology) 1/50, rabbit anti a 6 integrin (Santa Cruz Biotechnology) 1/50, rabbit anti Ki67 (Dako), and chicken anti DSG4 (custom raised by Washington Biotechnology) 1/200. The secondary antibodies used were swine anti rabbit (Dako) 1/100, and donkey anti chicken Cy3 (Jackson Immunoresearch laboratories) 1/800.

### REFERENCES

[1] J. P. Sundberg, and L. E. King, Jr., Mouse mutations as animal models and biomedical tools for dermatological research, J Invest Dermatol 106 (1996) 368-76.
[2] M. Nakamura, J. P. Sundberg, and R. Paus, Mutant laboratory mice with abnormalities in hair follicle morphogenesis, cycling, and/or structure: annotated tables, Exp Dermatol 10 (2001) 369-90.
[3] J. Palm, and F. G. Ferguson, Fuzzy, a hypotrichotic mutant in linkage group I of the Norway rat, J Hered 67 (1976) 284-8.
[4] A. N. Moemeka, A. L. Hildebrandt, P. Radaskiewicz, and T. R. King, Shorn (shn): a new mutation causing hypotrichosis in the Norway rat, J Hered 89 (1998) 257-60.
[5] M. S. Islam, L. Zhao, J. Zhou, L. Dong, J. N. McDougal, and G. L. Flynn, Systemic uptake and clearance of chloroform by hairless rats following dermal exposure: II. Absorption of the neat solvent, Am Ind Hyg Assoc J 60 (1999) 438-43.
[6] C. C. Sumian, F. B. Pitre, B. E. Gauthier, M. Bouclier, and S. R. Mordon, Laser skin resurfacing using a frequency doubled Nd: YAG laser after topical application of an exogenous chromophore, Lasers Surg Med 25 (1999) 43-50.
[7] W. Ahmad, M. F. ul Haque, V. Brancolini, H. C. Tsou, S. ul Haque, H. Lam, V. M. Aita, J. Owen, M. deBlaquiere, J. Frank, P. B. Cserhalmi-Friedman, A. Leask, J. A. McGrath, M. Peacocke, M. Ahmad, J. Ott, and A. M. Christiano, Alopecia universalis associated with a mutation in the human hairless gene, Science 279 (1998) 720-724.
8] J. P. Stoye, S. Fenner, G. E. Greenoak, C. Moran, and J. M. Coffin, Role of endogenous retroviruses as mutagens: the hairless mutation of mice, Cell 54 (1988) 383-91.
[9] A. A. Panteleyev, R. Paus, W. Ahmad, J. P. Sundberg, and A. M. Christiano, Molecular and functional aspects of the hairless (hr) gene in laboratory rodents and humans, Exp Dermatol 7 (1998) 249-67.
[10] J. Frank, C. Pignata, A. A. Panteleyev, D. M. Prowse, H. Baden, L. Weiner, L. Gaetaniello, W. Ahmad, N. Pozzi, P. B. Cserhalmi-Friedman, V. M. Aita, H. Uyttendaele, D. Gordon, J. Ott, J. L. Brissette, and A. M. Christiano, Exposing the human nude phenotype, Nature 398 (1999) 473-4.
[11] T. Schlake, M. Schorpp, A. Maul-Pavicic, A. M. Malashenko, and T. Boehm, Forkhead/winged-helix transcription factor Whn regulates hair keratin gene expression: molecular analysis of the nude skin phenotype, Dev Dyn 217 (2000) 368-76.
[12] X. Tong, and P. A. Coulombe, Mouse models of alopecia: identifying structural genes that are baldly needed, Trends Mol Med 9 (2003) 79-84.
[13] X. Montagutelli, A. Lalouette, H. J. Boulouis, J. L. Guenet, and J. P. Sundberg, Vesicle formation and follicular root sheath separation in mice homozygous for deleterious alleles at the balding (bal) locus, J Invest Dermatol 109 (1997) 324-8.
[14] L. Pulkkinen, W. W. Choi, A. Simpson, X. Montagutelli, J. Sundberg, J. Uitto, and M. G. Mahoney, Loss of cell adhesion in Dsg3bal-Pas mice with homozygous deletion mutation (2079de114) in the desmoglein 3 gene, J Invest Dermatol 119 (2002) 1237-43.
[15] X. Montagutelli, M. E. Hogan, G. Aubin, A. Lalouette, J. L. Guenet, L. E. King., and J. P. Sundberg, Lanceolate hair (lah): a recessive mouse mutation with alopecia and abnormal hair, J Invest Dermatol 107 (1996) 20-5.
[16] J. P. Sundberg, D. Boggess, C. Bascom, B. J. Limberg, S. L. D., S. B. A., K. L. E. Jr., and X. Montagutelli, Lanceolate hair-J (lahJ): a mouse model for human hair disorders, Exp Dermatol 9 (2000) 206-218.
[17] A. Kljuic, H. Bazzi, J. P. Sundberg, A. Martinez-Mir, R. O'Shaughnessy, M. G. Mahoney, M. Levy, X. Montagutelli, W. Ahmad, V. M. Aita, D. Gordon, J. Uitto, D. Whiting, J.
Ott, S. Fischer, T. C. Gilliam, C. A. Jahoda, R. J. Morris, A. A. Panteleyev, V. T. Nguyen, and A. M. Christiano, Desmoglein 4 in hair follicle differentiation and epidermal adhesion: evidence from inherited hypotrichosis and acquired pemphigus vulgaris, Cell 113 (2003) 249-60.
[18] W. He, P. Cowin, and D. L. Stokes, Untangling desmosomal knots with electron tomography, Science 302 (2003) 109-13.
[19] H. Posthaus, C. M. Dubois, M. H. Laprise, F. Grondin, M. M. Suter, and E. Muller, Proprotein cleavage of E-cadherin by furin in baculovirus “over-expression system: potential role of other convertases in mammalian cells, FEBS Lett 438 (1998) 306-10.
[20] M. G. Mahoney, A. Simpson, S. Aho, J. Uitto, and L. Pulkkinen, Interspecies conservation and differential expression of mouse desmoglein gene family, Exp Dermatol 11 (2002) 115-25.
[21] L. Pulkkinen, W. W. Choi, A. Kljuic, J. Uitto, and M. G. Mahoney, Novel member of the mouse desmoglein family: Dsgl-B, Exp Derm 12 (2003) 11-19.
[22] A. Kljuic, and A. M. Christiano, A novel mouse desmosomal cadherin family member, desmoglein lg, Exp Derm 12 (2003) 20-29.
[23] M. S. Springer, W. J. Murphy, E. Eizirik, and S. J. O'Brien, Placental mammal diversification and the Cretaceous-Tertiary boundary, Proc Natl Acad Sci USA 100 (2003) 1056-61.
[24] T. J. Boggon, J. Murray, S. Chappuis-Flament, E. Wong, B. M. Gumbiner, and L. Shapiro, C-cadherin ectodomain structure and implications for cell adhesion mechanisms, Science 296 (2002) 1308-13.
[25] B. Nagar, M. Overduin, M. Ikura, and J. M. Rini, Structural basis of calcium-induced E-cadherin rigidification and dimerization, Nature 380 (1996) 360-4.
[26] M. Ozawa, J. Engel, and R. Kemler, Single amino acid substitutions in one Ca2+ binding site of uvomorulin abolish the adhesive function, Cell 63 (1990) 1033-8.
[27] S. Pokutta, K. Herrenknecht, R. Kemler, and J. Engel, Conformational changes of the recombinant extracellular domain of E-cadherin upon calcium binding, Eur J Biochem 223 (1994) 1019-26.
[28] K. Tamura, W. S. Shan, W. A. Hendrickson, D. R. Colman, and L. Shapiro, Structure-function analysis of cell adhesion by neural (N-) cadherin, Neuron 20 (1998) 1153-63.
[29] I M. Freedberg, M. Tomic-Canic, M. Komine, and M. Blumenberg, Keratins and the keratinocyte activation cycle, J Invest Dermatol 116 (2001) 633-40.
[30] C. K. Jiang, T. Magnaldo, M. Ohtsuki, I. M. Freedberg, F. Bernerd, and M. Blumenberg, Epidermal growth factor and transforming growth factor alpha specifically induce the activation-and hyperproliferation-associated keratins 6 and 16, Proc Natl Acad Sci USA 90 (1993) 6786-90.
[31] J. M. Carroll, M. R. Romero, and F. M. Watt, Suprabasal integrin expression in the epidermis of transgenic mice results in developmental defects and a phenotype resembling psoriasis, Cell 83 (1995) 957-68.
[32] M. Kashiwagi, T. Kuroki, and N. Huh, Specific inhibition of hair follicle formation by epidermal growth factor in an organ culture of developing mouse skin, Dev Biol 189 (1997) 22-32.

### Example 3

A newly defined form of inherited hair loss, named localized autosomal recessive hypotrichosis (LAH, OMIM 607903), was recently described in the literature and shown to be linked to chromosome 18. We identified a large, intragenic deletion in the desmoglein 4 gene (DSG4) as the underlying mutation in two unrelated families of Pakistani origin. LAH is an autosomal recessive form of hypotrichosis affecting the scalp, trunk and extremities, and largely sparing the facial, pubic and axillary hair. Typical hairs are fragile and break easily, leaving short sparse scalp hairs with a characteristic appearance. Using comparative genomics, we also demonstrated that human LAH is allelic with the lanceolate hair (lah) mouse, as well as the lanceolate hair (lah) rat phenotype. In order to expand the allelic series of mutations in the desmoglein 4 gene underlying LAH in humans, we have begun molecular analysis of DSG4 in families from around the world.

Here, we describe the study of a family of Pakistani origin with two siblings affected with localized autosomal recessive hypotrichosis (LAH). The two affected children, a girl aged 5 years 9 months and a boy aged eighteen months, have two sisters with normal hair. Their parents, first cousins of Pakistani origin, are unaffected. They are part of a large family with extensive consanguinity but no other affected individuals. Both affected children were born without hair and neither infant was ritually shaved. Subsequently, sparse coarse hair growth was accompanied by itching, redness and roughness of the scalp. Both children are otherwise healthy and developing normally.

The findings on serial examination have been the same in both children. At the age of 2 months the proband showed complete alopecia with scalp follicular prominence. By 15 months there was sparse, coarse, brittle hair with follicular hyperkeratosis, erythema and scaling affecting particularly the scalp, but also eyebrows and eyelashes. Now aged 5 the girl's scalp hair remains sparse and is clearly brittle, less than 1 cm long at sites of friction and up to 8 cm in other areas. She now has marked follicular hyperkeratosis on the extensor aspects of the limbs. The skin is otherwise normal with no papular lesions on the limbs, and no palmoplantar keratoderma. Sweating, teeth and nails appear normal. The clinical findings are most consistent with a diagnosis of localized autosomal recessive hypotrichosis (LAH; OMIM#607903).

### EXPERIMENTAL RESULTS

We obtained DNA from the two affected individuals and both parents. Genomic DNA was isolated from peripheral blood collected in EDTA-containing tubes according to standard techniques (Sambrook et al 1989). All samples were collected following informed consent. To screen for a mutation in the human DSG4 gene, all exons and splice junctions were PCR amplified from genomic DNA and sequenced directly in an ABI Prism 310 Automated Sequencer, using the ABI Prism Big Dye Terminator Cycle Sequencing Ready Reaction Kit (PE Applied Biosystems, Foster City, Calif.), following purification in-Centriflex™ Gel Filtration Cartridges (Edge Biosystems, Gaithersburg, Md.) as we described herein. The mutation was identified by visual inspection and comparison with control sequences generated from unrelated, unaffected individuals. The deletion mutation is identified by the failure to PCR amplify exons 5, 6, 7 and 8 from homozygous affected individuals, followed by PCR and direct sequencing of the breakpoints in the surrounding introns.

A molecular analysis of the DSG4 gene was carried out in the family. The two affected siblings belong to a consanguineous pedigree with first-cousin parents. PCR amplification of exons 4 through 9 revealed the absence of amplification of exons 5-8 in the two patients (I-1 and II-2) whereas PCR bands of correct size were obtained from the parents' genomic DNA (I-1 and I-2). When using a forward PCR primer in intron 4 and a reverse primer in intron 8, a novel PCR fragment was obtained in all family members, corresponding to the deletion allele. Sequence analysis of the PCR products revealed a homozygous deletion encompassing exons 5 through 8 in the two affected individuals. Both parents are heterozygous for a wild-type and a deletion allele.

The deletion in DSG4 begins 35 bp upstream of exon 5 (within intron 4) and ends 289 bp downstream of exon 8 (within intron 8). This results in an in-frame deletion, leading to an internally truncated protein missing amino acids 125-335. These amino acids correspond to part of the EC1 domain, all of EC2 and the beginning of the EC3 domain. These regions of DSG4 are believed to be critical in cadherin-cadherin interaction and dimerization (Boggon et al. 2002) necessary for proper cell-cell adhesion.

Dsg4 is expressed in the inner epithelial layers of the hair follicle, where its function appears to be crucial during differentiation of the hair follicle layers. The significance of properly orchestrated adhesion during hair follicle development is underscored by several human disorders that result from mutations in adhesion plaque genes. The desmosomal plaque is composed of proteins from three different protein families, the desmosomal cadherin, plakin and armadillo families. Mutations in genes encoding proteins in all three families have been shown to result in disorders-of skin and hair follicle. For example, mutations in desmoplakin and plakoglobin, members of plakin and armadillo families respectively, underlie Naxos disease (OMIM 601214, 605676). Naxos disease is an autosomal recessive disorder characterized by wooly, sparse hair, keratoderma, and cardiomyopathy (McKoy et al. 2000; Norgett et al. 2000). Recessive mutations in plakophillin 1, another armadillo family member, result in ectodermal dysplasia with sparse hair and skin fragility (OMIM 604536) (McGrath et al. 1997). Interestingly, DSG4 is the only desmosomal cadherin, thus far, which has been associated with human hair phenotype. To date, no diseases have been described resulting from mutations in desmocollins and the dominant mutations identified in DSG1 result in striate palmoplantar keratoderma (OMIM 148700), characterized by thickening of the skin on palms and soles but no hair involvement. Furthermore, no human mutations have been found in DSG2 or DSG3 genes although mutations in the mouse Dsg3 result in the balding phenotype, characterized by cyclical hair loss (Koch et al. 1997; Pulkkinen et al. 2002).

It is not surprising that mutations in molecules that regulate desmosomal function can also give rise to related skin and hair phenotypes. Hailey-Hailey disease (HHD) (OMIM 604384) and Darier (DD) (OMIM 124200) disease which affect calcium pumps both present with loss of epidermal cell adhesion, acantholysis, and abnormal keratinization (Hu et al 2000; Sakuntabhai et al 1999). Furthermore, mutations in the components of the desmosome attached cytoskeleton, such as the IF keratin genes, hHb6 and hHbl, lead to the hair dystrophy disease, monilethrix (OMIM 158000) (Korge et al. 1998).

Mutations in P-cadherin, a member of the classical cadherin family and a component of adherent junctions, another type of adhesion plaque, have also been shown to result in hypotrichosis with fragile, beaded shafts and macular dystrophy (Indelman et al. 2002; Sprecher et al. 2001). It is interesting to note that one of the mutations described for P-cadherin is a missense mutation of a conserved residue within the fourth extracellular domain (Radice et al. 1997). All cadherins share a high level of homology with respect to protein domain organization. Each cadherin consist of five extracellular repeat domains (EC1-5), the transmembrane region, and the intracellular tail. The observation that mutations in the EC domains in both desmosomal and classical cadherins lead to comparable hypotrichosis phenotype underscores the functional similarity of the two proteins as well as the critical role of EC domains in epithelial adhesion.

We have identified the same deletion of exons 5-8 in the DSG4 gene in two Pakistani families, one residing in the US. Recent reports of three additional Pakistani families (Rafique et al. 2003) with LAH-like features and linked to chromosome 18, also suggest that DSG4 mutations underlie the disease in these families as well. Here, we report the identification of a LAH pedigree in the United Kingdom. There is a large Pakistani population in the UK, therefore this report should raise the awareness of LAH as a differential diagnosis to clinicians in this part of the world. Interestingly, the propagation of the identical EX5_{—}8del desmoglein 4 mutation in Pakistani families throughout widespread geographic regions suggests that this allele represents an ancestral mutation that has been widely dispersed.

### REFERENCES

Boggon T J, Murray J, Chappuis-Flament S, Wong E, Gumbiner B M, Shapiro L: C-cadherin ectodomain structure and implications for cell adhesion mechanisms. Science 296: 1308-1313, 2002.
Hu Z, Bonifas J M, Beech J et al: Mutations in ATP2C1, encoding a calcium pump, cause Hailey-Hailey disease. Nat Genet 24: 61-65, 2000.
Huber O: Structure and function of desmosomal proteins and their role in development and disease. Cell Mol Life Sci 60: 1872-1890, 2003.
Indelman M, Bergman R, Lurie R et al: A missense mutation in CDH3, encoding P-cadherin, causes hypotrichosis with juvenile macular dystrophy. J Invest Dermatol 119: 1210-1213, 2002.
Jahoda C A B, Kljuic A, O'Shaughnessy R et al : The lanceolate hair rat phenotype results from a missense mutation in a calcium coordinating site of the desmoglein 4 gene. Genomics, (in press).
Kljuic A, Bazzi H, Sundberg J P et al : Desmoglein 4 in hair follicle differentiation and epidermal adhesion: evidence from inherited hypotrichosis and acquired pemphigus vulgaris. Cell 113: 249-260, 2003a.
Kljuic A, Gilead L, Martinez-Mir A, Frank J, Christiano A M, Zlotogorski A: A Nonsense Mutation in the Desmoglein 1 Gene Underlies Striate Keratoderma. Exp Dermatol 12: 523-527, 2003b.
Koch P J, Mahoney M G, Ishikawa H et al : Targeted disruption of the pemphigus vulgaris antigen (desmoglein 3) gene in mice causes loss of keratinocyte cell adhesion with a phenotype similar to pemphigus vulgaris. J Cell Biol 137: 1091-1102, 1997.
Korge B P, Healy E, Munro C S et al: A mutational hotspot in the 2B domain of human hair basic keratin 6 (hHb6) in monilethrix patients. J Invest Dermatol 111: 896-899, 1998.
McGrath J A, McMillan J R, Shemanko C S et al : Mutations in the plakophilin 1 gene result in ectodermal dysplasia/skin fragility syndrome. Nat Genet 17: 240-244, 1997.
McKoy G, Protonotarios N, Crosby A et al: Identification of a deletion in plakoglobin in arrhythmogenic right ventricular cardiomyopathy with palmoplantar keratoderma and woolly hair (Naxos disease). Lancet 355: 2119-2124, 2000.
Norgett E E, Hatsell S J, Carvajal-Huerta L et al: Recessive mutation in desmoplakin disrupts desmoplakin-intermediate filament interactions and causes dilated cardiomyopathy, woolly hair and keratoderma. Hum Mol Genet 9: 2761-2766, 2000.
Pulkkinen L, Choi Y W, Simpson A, Montagutelli X, Sundberg J, Uitto J, Mahoney M G: Loss of cell adhesion in Dsg3bal-Pas mice with homozygous deletion mutation (2079de114) in the desmoglein 3 gene. J Invest Dermatol 119: 1237-1243, 2002.
Radice G L, Ferreira-Cornwell M C, Robinson S D, Rayburn H, Chodosh L A, Takeichi M, Hynes R O: Precocious mammary gland development in P-cadherin-deficient mice. J Cell Biol 139: 1025-1032, 1997.
Rafique M A, Ansar M, Jamal S M et al: A locus for hereditary hypotrichosis localized to human chromosome 18q21. 1. Eur J Hum Genet 11: 623-628, 2003.
Rickman L, Simrak D, Stevens H P et al : N-terminal deletion in a desmosomal cadherin causes the autosomal dominant skin disease striate palmoplantar keratoderma. Hum Mol Genet 8: 971-976, 1999.
Sakuntabhai A, Ruiz-Perez V, Carter S et al : Mutations in ATP2A2, encoding a Ca2+ pump, cause Darier disease. Nat Genet 21: 271-277, 1999.
Sambrook J, Fritsch E F, Maniatis T. 1989. Molecular cloning: a laboratory manual. Cold Spring Harbor Laboratory Press, New York.
Sprecher E, Bergman R, Richard G et al: Hypotrichosis with juvenile macular dystrophy is caused by a mutation in CDH3, encoding P-cadherin. Nat Genet 29: 134-136,2001.

I claim:

1. A catalytic deoxyribonucleic acid molecule that specifically cleaves a mRNA encoding desmoglein 4 comprising:
(a) a catalytic domain that cleaves mRNA at a defined consensus sequence;
(b) a binding domain contiguous with the 5 ′ end of the catalytic domain; and
(c) a binding domain contiguous with the 3 ′ end of the catalytic domain,
wherein the binding domains are complementary to, and therefore hybridize with, the two regions flanking the defined consensus sequence within the mRNA encoding desmoglein 4 at which cleavage is desired, and wherein each binding domain is at least 4 residues in length and both binding domains have a combined total length of at least 8 residues.

2. The catalytic deoxyribonucleic acid molecule of claim 1, wherein the catalytic domain has the sequence ggctagctacaacga (SEQ ID NO: 5), and cleaves mRNA at the consensus sequence purine: pyrimidine.

3. A catalytic ribonucleic acid molecule that specifically cleaves a mRNA encoding desmoglein 4 comprising:
(a) a catalytic domain that cleaves mRNA at a defined consensus sequence;
(b) a binding domain contiguous with the 5 ′ end of the catalytic domain; and
(c) a binding domain contiguous with the 3 ′ end of the catalytic domain,
wherein the binding domains are complementary to, and therefore hybridize with, the two regions flanking the defined consensus sequence within the mRNA encoding desmoglein 4 at which cleavage is desired, and wherein each binding domain is at least 4 residues in length and both binding domains have a combined total length of at least 8 residues.

4. The catalytic ribonucleic acid molecule of claim 3, wherein the catalytic domain has the sequence ctgatgagtccgtgaggacgaaaca (SEQ ID NO: 6), and cleaves mRNA at the consensus sequence 5′-NUH-3′, where N is any nucleotide, U is uridine and H is any nucleotide except guanine.

5. The catalytic ribonucleic acid molecule of claim 3, wherein the molecule is a hammerhead ribozyme or hairpin ribozyme.

6. The catalytic nucleic acid molecule of claim 1 or 3, wherein the mRNA encoding desmoglein 4 comprises consecutive nucleotides having the sequence set forth in SEQ ID NO: 2 or SEQ ID NO: 4.

7. The catalytic nucleic acid molecule of claim 1 or 3, wherein the desmoglein 4 comprises consecutive amino acids having the sequence set forth in SEQ ID NO: 1.

8. The catalytic nucleic acid molecule of claim 1 or 3, wherein the desmoglein 4 comprises consecutive amino acids having the sequence set forth in SEQ ID NO: 3.

9. The catalytic nucleic acid molecule of claim 1 or 3, wherein the cleavage site within the mRNA encoding desmoglein 4 is located within the first 3000 residues following the mRNA's 5′ terminus.

10. The catalytic nucleic acid molecule of claim 9, wherein the cleavage site within the mRNA encoding desmoglein 4 is located within the first 1500 residues following the mRNA's 5′ terminus.

11. The catalytic nucleic acid molecule of claim 1 or 3, wherein the mRNA encoding desmoglein 4 is from a subject selected from the group consisting of human, monkey, rat and mouse.

12. A pharmaceutical composition comprising the catalytic nucleic acid molecule of claim 1 or 3 and a pharmaceutically acceptable carrier.

13. The pharmaceutical composition of claim 12, wherein the carrier is an alcohol.

14. The pharmaceutical composition of claim 13, wherein the carrier is ethylene glycol.

15. The pharmaceutical composition of claim 12, wherein the carrier is a liposome.

16. A method of specifically cleaving an mRNA encoding desmoglein 4 comprising contacting the mRNA with the catalytic nucleic acid molecule of claim 1 or 3 under conditions permitting the molecule to cleave the mRNA encoding desmoglein 4.

17. A method of specifically cleaving an mRNA encoding desmoglein 4 in a cell, comprising contacting the cell containing the mRNA with the catalytic nucleic acid molecule of claim 1 or 3 under conditions permitting the catalytic nucleic acid molecule to specifically cleave the mRNA encoding desmoglein 4 in the cell.

18. A method of specifically inhibiting the expression of desmoglein 4 in a cell that would otherwise express desmoglein 4, comprising contacting the cell with the catalytic nucleic acid molecule of claim 1 or 3 so as to specifically inhibit the expression of desmoglein 4 in the cell.

19. A method of specifically inhibiting the expression of desmoglein 4 in a subject's cells comprising administering to the subject an amount of the catalytic nucleic acid molecule of claim 1 or 3 effective to specifically inhibit the expression of desmoglein 4 in the subject's cells.

20. A method of specifically inhibiting the expression of desmoglein 4 in a subject's cells comprising administering to the subject an amount of the pharmaceutical composition of claim 12 effective to specifically inhibit the expression of desmoglein 4 in the subject's cells.

21. A method of inhibiting hair production by a hair-producing cell comprising contacting the cell with an effective amount of the catalytic nucleic acid of claim 1 or 3.

22. A method of inhibiting hair growth in a subject comprising administering to the subject an effective amount of the pharmaceutical composition of claim 12.

23. A method of inhibiting the transition of a hair follicle from proliferation to differentiation comprising contacting the follicle with an effective amount of the catalytic nucleic acid of claim 1 or 3.

24. A method of inhibiting the transition of a hair follicle from proliferation to the differentiation comprising contacting the follicle with an effective amount of the pharmaceutical composition of claim 12.

25. The method of claim 17, wherein the cell is a keratinocyte.

26. The method of claim 18, wherein the cell is a keratinocyte.

27. The method of claim 19, wherein the cell is a keratinocyte.

28. The method of claim 20, wherein the cell is a keratinocyte.

29. The method of claim 21, wherein the cell is a keratinocyte.

30. The method of claim 19, wherein the subject is a human.

31. The method of claim 20, wherein the subject is a human.

32. The method of claim 22, wherein the subject is a human.

33. The method of claim 19, wherein the catalytic nucleic acid molecule is administered topically.

34. The method of claim 33, wherein the catalytic nucleic acid is administered dermally.

35. The method of claim 20, wherein the pharmaceutical composition is administered topically.

36. The method of claim 35, wherein the pharmaceutical composition is administered dermally.

37. The method of claim 22, wherein the pharmaceutical composition is administered topically.

38. The method of claim 37, wherein the pharmaceutical composition is administered dermally.

39. A vector which comprises a sequence encoding the catalytic nucleic acid molecule of claim 1 or 3.

40. A host-vector system comprising a cell having the vector of claim 39 therein.

41. A method of producing the catalytic nucleic acid molecule of claim 1 or 3 comprising culturing a cell having therein a vector comprising a sequence encoding said catalytic nucleic acid molecule under conditions permitting the expression of the catalytic nucleic acid molecule by the cell.

42. A nucleic acid molecule that specifically hybridizes under conditions of high stringency to a mRNA encoding a desmoglein 4 so as to inhibit the translation thereof in a cell.

43. The nucleic acid of claim 42, wherein the nucleic acid is a ribonucleic acid.

44. The nucleic acid of claim 42, wherein the nucleic acid is deoxyribonucleic acid.

45. The nucleic acid molecule of claim 42, wherein the nucleic acid molecule is complementary to and hybridizes with a portion of the desmoglein 4-encoding mRNA, and is between 8 and 40 nucleobases in length.

46. The nucleic acid molecule of claim 42, wherein the desmoglein 4 comprises consecutive amino acids having the sequence set forth in SEQ ID NO: 1 or SEQ ID NO: 3.

47. The nucleic acid molecule of claim 42, wherein the mRNA encoding desmoglein 4 comprises consecutive nucleotides having the sequence set forth in SEQ ID NO: 2 or SEQ ID N:4.

48. A vector which comprises a sequence encoding the nucleic acid molecule of claim 42.

49. A host-vector system comprising a cell having the vector of claim 48 therein.

50. A pharmaceutical composition comprising
(a) the nucleic acid molecule of claim 42 or the vector of claim 48 and
(b) a pharmaceutically acceptable carrier.

51. The pharmaceutical composition of claim 50, wherein the carrier is an alcohol.

52. The pharmaceutical composition of claim 51, wherein the carrier is ethylene glycol.

53. The pharmaceutical composition of claim 50, wherein the carrier is a liposome.

54. A method of specifically inhibiting the expression of desmoglein 4 in a cell that would otherwise express desmoglein 4, comprising contacting the cell with the nucleic acid molecule of claim 42 so as to specifically inhibit the expression of desmoglein 4 in the cell.

55. A method of specifically inhibiting the expression of desmoglein 4 in a subject's cells comprising administering to the subject an amount of the nucleic acid molecule of claim 42 effective to specifically inhibit the expression of desmoglein 4 in the subject's cells.

56. A method of specifically inhibiting the expression of desmoglein 4 in a subject's cells comprising administering to the subject an amount of the pharmaceutical composition of claim 50 effective to specifically inhibit the expression of desmoglein 4 in the subject's cells.

57. A method of inhibiting hair production by a hair-producing cell comprising contacting the cell with an effective amount of the nucleic acid molecule of claim 42.

58. A method of inhibiting hair growth in a subject comprising administering to the subject an effective amount of the pharmaceutical composition of claim 50.

59. The method of claim 54, wherein the cell is a keratinocyte.

60. The method of claim 55, wherein the cell is a keratinocyte.

61. The method of claim 56, wherein the cell is a keratinocyte.

62. The method of claim 57, wherein the cell is a keratinocyte.

63. The method of claim 55, wherein the subject is a human.

64. The method of claim 56, wherein the subject is a human.

65. The method of claim 58, wherein the subject is a human.

66. The method of claim 55, wherein the nucleic acid molecule is administered topically.

67. The method of claim 66, wherein the nucleic acid is administered dermally.

68. The method of claim 56, wherein the pharmaceutical composition is administered topically.

69. The method of claim 68, wherein the pharmaceutical composition is administered dermally.

70. A method of producing the nucleic acid molecule of claim 42 comprising culturing a cell having therein a vector comprising a sequence encoding the nucleic acid molecule under conditions permitting the expression of the nucleic acid molecule by the cell.

71. A non-human transgenic mammal, wherein the mammal's genome:
(a) has stably integrated therein a nucleotide sequence encoding a human desmoglein 4 operably linked to a promoter, whereby the nucleotide sequence is expressed; and
(b) lacks an expressible endogenous desmoglein 4 encoding nucleic acid sequence.

72. An oligonucleotide comprising consecutive nucleotides that hybridizes with a desmoglein 4-encoding mRNA under conditions of high stringency and is between 8 and 40 nucleotides in length.

73. The oligonucleotide of claim 72, wherein the oligonucleotide inhibits translation of the desmoglein 4-encoding mRNA.

74. The oligonucleotide of claim 72, wherein at least one internucleoside linkage within the oligonucleotide comprises a phosphorothioate linkage.

75. The oligonucleotide of claim 72, wherein the nucleotides comprise at least one deoxyribonucleotide.

76. The oligonucleotide of claim 72, wherein the nucleotides comprise at least one ribonucleotide.

77. The oligonucleotide of claim 72, wherein the desmoglein 4-encoding mRNA encodes human desmoglein 4.

78. The oligonucleotide of claim 77, wherein the desmoglein 4-encoding mRNA comprises consecutive nucleotides, the sequence of which is set forth in SEQ ID NO: 2 or 4.

79. A pharmaceutical composition comprising the oligonucleotide of claim 72 and a pharmaceutically acceptable carrier.

80. A method of treating a subject which comprises administering to the subject an amount of the oligonucleotide of claim 72 effective to inhibit expression of a desmoglein 4 in the subject so as to thereby treat the subject.

81. A method of specifically inhibiting the expression of desmoglein 4 in a cell that would otherwise express desmoglein 4, comprising contacting the cell with the oligonucleotide of claim 72 so as to specifically inhibit the expression of desmoglein 4 in the cell.

82. A method of specifically inhibiting the expression of desmoglein 4 in a subject's cells comprising administering to the subject an amount of the oligonucleotide of claim 72 effective to specifically inhibit the expression of desmoglein 4 in the subject's cells.

83. A method of specifically inhibiting the expression of desmoglein 4 in a subject's cells comprising administering to the subject an amount of the pharmaceutical composition of claim 79 effective to specifically inhibit the expression of desmoglein 4 in the subject's cells.

84. A method of inhibiting hair production by a hair-producing cell comprising contacting the cell with an effective amount of the oligonucleotide of claim 72.

85. A method of inhibiting hair growth in a subject comprising administering to the subject an effective amount of the pharmaceutical composition of claim 79.

86. The method of claim 80, 81, 82, 83, 84, or 85, wherein the subject is a mammal.

87. The method of claim 86, wherein the mammal is a human being.


SEQUENCE LISTING (40 sequences)

SEQ ID NO: 1 - protein, 1040 aa, Homo sapiens
  Homo sapiens Desmoglein 4
MDWLFFRNICLLIILMVVMEVNSEFIVEVKEFDIENGTTKWQTVRRQKREWIKFAAACREGEDNSKRNPIAKIRSDCESNQKITYRISGVGIDRPPYGVFTINPRTGEINITSVVDREITPLFLIYCRALNSRGEDLERPLELRVKVMDINDNAPVFSQSVYTASIEENSDANTLVVKLCATDADEENHLNSKIAYKIVSQEPSGAPMFILNRYTGEVCTMSSFLDREQHSMYNLVVRGSDRDGAADGLSSECDCRIKVLDVNDNFPTLEKTSYSASIEENCLSSELIRLQAIDLDEEGTDNWLAQYLILSGNDGNWFDIQTDPQTNEGILKVVKMLDYEQAPNIQLSIGVKNQADFHYSVASQFQMHPTPVRIQVVDVREGPAFHPSTMAFSVREGIKGSSLLNYVLGTYTAIDLDTGNPATDVRYIIGHDAGSWLKIDSRTGEIQFSREFDKKSKYIINGIYTAEILAIDDGSGKTATGTICIEVPDINDYCPNIFPERRTICIDSPSVLISVNEHSYGSPFTFCVVDEPPGIADMWDVRSTNATSAILTAKQVLSPGFYEIPILVKDSYNRACELAQMVQLYACDCDDNHMCLDSGAAGIYTEDITGDTYGPVTEDQAGVSNVGLGPAGIGMMVLGILLLILAPLLLLLCCCKQRQPEGLGTRFAPVPEGGEGVMQSWRIEGAHPEDRDVSNICAPMTASNTQDRMDSSEIYTNTYAAGGTVEGGVSGVELNTGMGTAVGLMAAGAAGASGAARKRSSTMGTLRDYADADINMAFLDSYFSEKAYAYADEDEGRPANDCLLIYDHEGVGSPVGSIGCCSWIVDDLDESCMETLDPKFRTLAEICLNTEIEPFPSHQACIPISTDLPLLGPNYFVNESSGLTPSEVEFQEEMAASEPVVHGDIIVTETYGNADPCVQPTTIIFDPQLAPNVVVTEAVMAPVYDIQGNICVPAELADYNNVIYAERVLASPGVPDMSNSSTTEGCMGPVMSGNILVGPEIQVMQMMSPDLPIGQTVGSTSPMTSRHRVTRYSNIHYTQQ

SEQ ID NO: 2 - DNA, 3123 bp, Homo sapiens
  Homo sapiens Desmoglein 4 cDNA
atggattggctcttcttcagaaacatttgccttttgatcattctaatggtggtgatggaagtaaacagtgaatttattgttgaggtgaaggaatttgacattgaaaatggcactacaaaatggcaaacagtcagaagacaaaagcgggagtggatcaagtttgccgcagcctgtcgagaaggagaggacaactcgaagaggaaccccattgccaaaattcgatcagactgcgaatcgaaccagaagataacataccggatttctggagtagggattgatcgaccaccatatggggtattcaccattaatcctcgcactggggaaattaacatcacttcagtggtagacagagaaataactccacttttcttgatctattgccgggctctgaattcacggggtgaagatttagaaaggcctcttgagcttagagtcaaagttatggacataaatgataacgctccagtcttttcgcaaagtgtatacacagccagcattgaagaaaatagtgatgccaatacattggtagtaaagttatgtgccacagatgcagatgaagaaaatcatctgaattctaaaattgcctacaagatcgtctctcaggagccatcaggtgcacccatgttcattctgaataggtacactggagaagtctgcaccatgtccagtttcttggacagagagcaacacagtatgtacaacctggttgtgagaggctcagatcgggatggagctgcagatggactgtcttctgagtgtgactgtagaatcaaggttttagacgtcaacgataatttccccaccttagagaaaacttcatactcagccagtattgaagagaattgtttaagttcggaactgatacgattacaagcaattgatcttgatgaagaaggcactgataactggttggctcaatatttaattctctctggaaatgatgggaattggttcgatattcaaacagatccacaaaccaatgaaggcattttgaaagttgtcaagatgctggattatgaacaagcacctaacattcagcttagtatcggagttaaaaaccaagctgattttcactactccgttgcttctcaattccaaatgcacccaacccctgtgagaattcaagttgttgatgtgagagaaggacctgcatttcatccaagtactatggcttttagtgtgcgggaaggaataaaaggaagttccttattgaattatgtgcttggcacatatacagccatagatttggacacaggaaaccctgcaacagatgtcagatatatcatagggcatgatgcaggcagctggttaaaaattgattcaagaactggtgagatacaattttctagagaatttgataagaagtcaaaatatattatcaatgggatatacacagcagagatcctggctatagatgatggctctggaaaaacagctacaggaaccatatgtattgaggttcctgatatcaatgattattgtccaaacatttttcctgaaagaagaaccatctgcattgactctccatcagtccttatctctgttaatgaacattcttatgggtctccgtttactttctgtgttgttgatgagccaccaggaatagctgacatgtgggatgtcagatcaacaaatgctacctcggcaatccttacggctaagcaggttttatctccaggattttatgaaatcccaatcctggtgaaggacagctataacagagcatgtgaattggcacaaatggtgcagttatatgcctgtgattgcgatgacaaccacatgtgcctggactctggtgccgcgggcatctacacagaggacataactggtgacacgtatgggcctgtcactgaagaccaagctggagtttcaaatgttggtcttggaccagcagggattggcatgatggttctgggcatcctgctactgattttggctccactcttgctgctcctgtgttgctgcaaacagagacagccagaaggcctgggaacaagatttgctcctgtgcctgagggcggagaaggagtgatgcagtcttggagaattgaaggggcccatcccgaggacagggatgtgtcaaatatatgtgcacccatgacagcctcaaatacccaggatcggatggattcctctgaaatctacaccaacacctatgcagccgggggcacggtggaaggaggtgtatcgggagtggagctcaacacaggtatggggacagccgttggcctcatggccgcaggggccgcaggagcctcaggggccgcaaggaagaggagctctaccatgggaaccctgcgggactacgctgacgcagacatcaacatggctttcttggacagctacttctcggagaaagcgtatgcttatgcagatgaagatgaaggtcgaccagccaatgactgcttgctcatttatgaccacgagggagtcgggtctcccgtaggctctattggttgttgcagttggattgtggatgacttagatgaaagctgcatggaaactttagatccaaaatttaggactcttgctgagatctgcttaaacacagaaattgaaccatttccttcacaccaggcttgtataccaatcagtactgacctccctttgctcggacctaattactttgttaatgaatcttcaggattgactccctcagaagttgaattccaagaagaaatggcagcatctgaacccgtggtccatggggatattattgtgactgagacttacggtaatgctgatccatgtgtgcaacccactacaattatttttgatcctcagcttgcacccaatgttgtagtaaccgaagcagtaatggcacctgtctatgatattcaagggaatatttgtgtacctgctgagttagcagattacaacaatgtaatctatgctgagagagtactggctagtcctggtgtgcctgacatgagcaatagtagcacgactgagggttgtatgggacctgtgatgagcggcaatattttagtagggccagaaattcaagtgatgcaaatgatgagtccagaccttcccataggccaaaccgttggctccacatcccccatgacatctcgacacagagtaacacgatacagtaacatacattacacccaacagtaa

SEQ ID NO: 3 - protein, 1041 aa, Mus musculus
  Mus musculus  Desmoglein 4
MDWLLFRNICLLILFMVVLGVNSEFIVEVKELDIENGTTTWQTVRRQKREWIKFAAACREGEDNSKRNPIARIRSDCEVSQRITYRISGAGIDRPPYGVFTINPRTGEINITSVVDREITPLFLIHCRALNSRGEDLERPLELRVKVMDVNDNPPVFTQNVYTANIEENSDANALVVKLSATDADEDNHLNSKIAYKIISQEPAGAPMFMVNRYTGEVRTMSNFLDREQHSMYNLLVRGSDRDGATDGLSSECDCRIKILDVNDNFPILEKTSYSASIEENCLSSELIRLQAIDLDEEGTDNWLAQYSILSGNDGNWFEIQTDPKTNEGILKVVKMLDYEQEPNIYLSIGVRNQAEFHHSVASQFQMHSTPVRIQVVNVREGPTFSPSSMTFSLRGGMRGASLMNYVLGTYTAIDMDTGNPATNVRYVIGHDAGSWLKVDSRTGEIQFSREFDMKSKYITDGIYAAQILAIDDGSGRTATGTICIEIPDANDYCPVIYAESRSVCTHASSVRIYVNDHSFGSPFTFCVVDESPDIANIWDIRSINGTSAILMTEQTLSPGPYQIPILVKDSHNRACELPQTVLLDACFCDDHHVCLHSSTTGIYTGDITWVTDDMYGTVTDDGVRQSNVGLGPAGIGMIILGLLLLLLSPLLLLMCCCKRRQPEGLGTRFAPVPEGGEGVMQPWRIEGAHPEDRDVSNICVPMTASNTQDRIDSSEIYTNTYAGGGTVEGGVSGVELNTGVGTATGMVAAGATGTLRKRSSTIGTLREYQDTGMNMAFLDSYFSEKAYAYADEDEGRPANDCLLIYDHEGAGSPVGSIGCCSWIVDDLDESYIETLDPKFRTLAEICLDTEIEPFPSHQACIPISTDLPLLGPNYFVNESSGMTLSEAEFQAEMAAASEPMIHGDIIVTETYTTSDPCVQPTTIVFDSQIPPNVVVTETVMAPVYDVQGNICVPAEIANTHNVYYAERVVASPGIPDMGNSNISDACIGPVMSGGILVGPEIQVTQMMSPDIHISQTTGSTSPMTSQHRVTRYSNMHYSRQ

SEQ ID NO: 4 - DNA, 3126 bp, Mus musculus
  Mus musculus Desmoglein 4 cDNA
atggactggcttctcttcagaaatatttgccttctgatcttatttatggtggtactgggagtaaacagtgaatttatcgttgaggtaaaagaattagacattgaaaatggcacaacaacctggcagactgtcagaagacaaaagcgggagtggatcaagtttgcggcagcctgtcgggagggcgaggacaactccaagaggaatccaattgccagaattagatcagactgtgaagtaagccagaggataacctatcgaatctctggagcggggattgatcggccaccttacggagtgttcaccatcaaccctcggactggagaaatcaatattacctcagtggtggacagagagataaccccactgttcctgatccattgccgagctctcaactcccgaggagaagacttggagagaccactggagctcagagtgaaagtcatggatgtgaacgacaaccccccagtcttcactcagaatgtgtacacagccaacattgaagagaacagcgacgccaatgcactggtggtaaagttaagtgccacagatgcagatgaagacaatcacttgaattctaaaattgcatacaagatcatctcccaggagcctgctggtgcacccatgttcatggtgaacaggtacactggagaagtccgcacgatgtccaatttccttgacagagagcaacacagtatgtacaatctactggtgaggggctcagatcgggatggagccacagatgggctgtcctctgaatgtgactgcagaatcaagattttggatgtcaatgataatttccccatcttggagaagacctcatactcagcaagtattgaagaaaattgtttgagctcagaactgatacgactacaagcaatcgatctggatgaagaaggcactgataactggttggcacaatactccatcctctctggcaatgatgggaactggtttgaaatccaaacagatccaaaaaccaatgaaggcattttgaaagtggtcaagatgctggattatgaacaagaacctaacatttacctcagcattggagttagaaaccaagctgagtttcaccactcagtagcatctcaattccaaatgcactctacccctgtgagaatccaggttgttaacgtgcgggaaggaccgacctttagcccaagttccatgacatttagtctacgaggaggaatgagaggagcctccttaatgaactatgtgcttggcacatacacagccatagatatggacacaggaaaccctgcaaccaatgtcaggtatgtcatagggcatgatgcaggcagctggttaaaagttgattcaaggactggtgagatacagttttctagggaatttgacatgaagtcaaaatatattacggatgggatatatgcagcacagatcctggctatagatgatggctctgggagaacagctacaggaaccatatgcattgagattcctgatgccaacgattactgtcccgtcatttatgctgaaagcaggagtgtctgcacccatgcttcgtctgtcaggatctatgtcaacgatcattctttcgggtctccctttaccttttgtgtagttgatgagtcaccagacatagctaatatatgggatatcaggtccatcaatggaacctctgcaatccttatgactgagcagactttgtctccaggaccctaccaaatccccatcctcgtgaaggacagtcacaaccgagcatgtgaattgccacagacagtgctgttagatgcctgcttttgtgatgaccaccatgtgtgcttgcactccagtaccacgggcatctacactggagacatcacctgggtgactgatgacatgtatgggactgtcactgatgatggagttaggcagtcaaacgtcggacttggcccagcagggattggcatgattattttggggctcttgctactgctcttgtcaccccttttactgctcatgtgttgctgcaagcgaagacagccagaaggcctggggacaaggtttgcccctgtgcctgagggaggcgaaggagtgatgcagccctggagaatagagggggctcatccagaagacagggatgtatcaaatatttgtgtacccatgacagcctctaatacccaagatcgcattgattcatctgaaatctacaccaatacttacgctggtggaggaacagtggaagggggcgtgtcaggagtggagctcaatacaggtgttgggacagccacaggcatggtggctgcaggagcaacagggaccctaaggaagaggagttcaactatagggactcttcgggaataccaagacactggcatgaacatggccttcctggacagctacttctccgagaaagcgtatgcatatgcagatgaagatgaaggccgaccagccaatgactgcctgctcatttatgatcacgaaggagctgggtcacccgtgggctccattggctgctgcagttggattgtggatgacttagacgaaagctacatagagactttggatccaaagttcaggactctagctgaaatctgcttggacacagagattgaacccttcccttcacaccaggcctgtataccaatcagcactgacctccctctgctggggcctaattactttgtcaatgagtcttcaggaatgactctctcagaggctgaattccaagcagaaatggcagcagcctctgaacccatgattcatggggacatcatagtgactgagacctacactacttctgacccatgtgtccaacccacaacgattgtctttgactctcagattccaccaaacgtagtcgtgaccgaaaccgtaatggcacctgtctacgatgttcaagggaacatttgtgtgcctgctgagatagccaacacacacaatgtatactatgctgagagagtggtggctagccctggtatacctgacatgggcaacagtaacattagtgatgcatgtataggaccagtgatgagcggcggtattttggtagggccagaaattcaagtgacacaaatgatgagcccagacattcacataagccaaaccacaggctccacctccccaatgacgtctcaacaccgagtaaccagatacagcaatatgcactactcccgacagtaa

SEQ ID NO: 5 - DNA, 15 bp, Artificial Sequence
  synthetic catalytic domain
ggctagctacaacga

SEQ ID NO: 6 - DNA, 25 bp, Artificial Sequence
  synthetic catalytic domain
ctgatgagtccgtgaggacgaaaca

SEQ ID NO: 7 - DNA, 20 bp, Artificial Sequence
  synthetic primer
tctcctagtacagcctgctt

SEQ ID NO: 8 - DNA, 20 bp, Artificial Sequence
  synthetic primer
agtggtctctccaagtcttc

SEQ ID NO: 9 - DNA, 20 bp, Artificial Sequence
  synthetic primer
agtttgccgcagcctgtcga

SEQ ID NO: 10 - DNA, 21 bp, Artificial Sequence
  synthetic primer
ccagttatcagtgccttcttc

SEQ ID NO: 11 - DNA, 19 bp, Artificial Sequence
  synthetic primer
ttgatcggccaccttacgg

SEQ ID NO: 12 - DNA, 20 bp, Artificial Sequence
  synthetic primer
ccaaccagttatcagtgcct

SEQ ID NO: 13 - protein, 22 aa, Artificial Sequence
  Synthetic Desmoglein 4 Peptide
NATSAILTALQVLSPGFYEIPI

SEQ ID NO: 14 - protein, 5 aa, Artificial Sequence
  synthetic motif
RXRKR

SEQ ID NO: 15 - protein, 3 aa, Artificial Sequence
  synthetic tripeptide sequence
RAL

SEQ ID NO: 16 - protein, 5 aa, Artificial Sequence
  synthetic calcium binding sites
DXNDN

SEQ ID NO: 17 - protein, 5 aa, Artificial Sequence
  synthetic calcium binding site
XXDXD

SEQ ID NO: 18 - protein, 3 aa, Artificial Sequence
  synthetic site for N-linked glycosylation
NXX

SEQ ID NO: 19 - protein, 6 aa, Artificial Sequence
  synthetic core repeat sequence in Rattus Desmoglein 4
DIIVTE

SEQ ID NO: 20 - protein, 6 aa, Artificial Sequence
  synthetic core repeat sequence in Rattus Desmoglein 4
NVVVTE

SEQ ID NO: 21 - protein, 6 aa, Artificial Sequence
  synthetic core repeat sequence in Rattus Desmoglein 4
NVIYAE

SEQ ID NO: 22 - protein, 6 aa, Artificial Sequence
  synthetic core repeat sequence in Mus Desmoglein 4
NVYYAE

SEQ ID NO: 23 - protein, 1049 aa, Artificial Sequence
  synthetic DSG1 from Homo sapiens
MDWSFFRVVAVLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 24 - protein, 1117 aa, Artificial Sequence
  synthetic DSG2 from Homo sapiens
MARTRDRVRLLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCEVLHGSGCREAQHDSYVGLGPAAIALMILAFLLLLLVPLLLLMCHCGKGAKAFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 25 - protein, 999 aa, Artificial Sequence
  synthetic DSG3 from Homo sapiens
MMGLFPRTTGALAIFVVVILVHGELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSASGSVQPAVSIPDPLQHGNYLVTETYSASGSLVQPSTAGFDPLLTQNVIVTERVICPISSVPGNLAGPTQLRGSHTMLCTEDPCSRLI

SEQ ID NO: 26 - DNA, 21 bp, Artificial Sequence
  Synthetic from WT/WT Mus
acagatgggctgtcctctgaa

SEQ ID NO: 27 - protein, 7 aa, Artificial Sequence
  synthetic from WT/WT Mus
TDGLSSE

SEQ ID NO: 28 - DNA, 21 bp, Artificial Sequence
  Synthetic from lahJ/lahJ Mus
acagatgggcttgtcctctga

SEQ ID NO: 29 - protein, 6 aa, Artificial Sequence
  synthetic from lahJ/lahJ Mus
TDGLVL

SEQ ID NO: 30 - protein, 15 aa, Artificial Sequence
  Synthetic from Dsg4
LNSKIAYKIISQEPA

SEQ ID NO: 31 - protein, 15 aa, Artificial Sequence
  Synthetic from DSG4
LNSKIAYKIVSQEPS

SEQ ID NO: 32 - protein, 15 aa, Artificial Sequence
  Synthetic from Dsg2
LNSKISYRIVSQEPA

SEQ ID NO: 33 - protein, 15 aa, Artificial Sequence
  Synthetic from Dsc1
LHTRLKYKILQQIPD

SEQ ID NO: 34 - protein, 15 aa, Artificial Sequence
  Synthetic from Dsc2
MHTRLRYSILEQSPS

SEQ ID NO: 35 - protein, 15 aa, Artificial Sequence
  Synthetic from Dsc3
MHTRLKYSILEQTPP

SEQ ID NO: 36 - protein, 15 aa, Artificial Sequence
  Synthetic from E-cad
YNAAIAYTIVSQDPE

SEQ ID NO: 37 - protein, 15 aa, Artificial Sequence
  Synthetic from N-cad
LNGMLRYRILSQAPS

SEQ ID NO: 38 - DNA, 6 bp, Artificial Sequence
  synthetic mutations
ctgtcc

SEQ ID NO: 39 - DNA, 7 bp, Artificial Sequence
  synthetic mutations
cttgtcc

SEQ ID NO: 40 - protein, 4 aa, Artificial Sequence
  synthetic sequence
LDRE